SEQUENCE LISTING (195 sequences)

SEQ ID NO: 1 - protein, 40 aa, synthetic construct
  VARIANT 2..5: note = G, A, V, I, P, D, R, K, Y, W, N or S
  VARIANT 7: note = G, A, V, I, P, D, R, K, Y, W, N or S
  VARIANT 9..13: note = G, A, V, I, P, D, R, K, Y, W, N or S
  VARIANT 15..17: note = G, A, V, I, P, D, R, K, Y, W, N or S
  VARIANT 24..25: note = G, A, V, I, P, D, R, K, Y, W, N or S
  VARIANT 28: note = G, A, V, I, P, D, R, K, Y, W, N or S
  VARIANT 30..31: note = G, A, V, I, P, D, R, K, Y, W, N or S
  VARIANT 34..35: note = G, A, V, I, P, D, R, K, Y, W, N or S
CXXXXFXCXXXXXCXXXTWVCDGXXDCXDXXDEXXCTPTP

SEQ ID NO: 2 - protein, 98 aa, synthetic construct
  VARIANT 76..84: note = G, A, V, I, P, D, R, K, Y, W, N or S
ATGVRAVPGNENSLEIEELARFAVDEHNKKENALLEFVRVVKAKEQVVAGTMYYLTLEAKDGGKKKLYEAKVWVKXXXXXXXXXNFKELQEFKPVGDA

SEQ ID NO: 3 - protein, 103 aa, synthetic construct
  VARIANT 47..55: note = A, D, G, I, K, N, P, R, S, V, W or Y
  VARIANT 81..89: note = G, A, V, I, P, D, R, K, Y, W, N or S
ATGVRAVPGNENSLEIEELARFAVDEHNKKENALLEFVRVVKAKEQXXXXXXXXXTMYYLTLEAKDGGKKKLYEAKVWVKXXXXXXXXXNFKELQEFKPVGDA

SEQ ID NO: 4 - protein, 98 aa, synthetic construct
  VARIANT 7..8: note = A, D, G, I, K, N, P, R, S, V, W or Y
  VARIANT 13: note = A, D, G, I, K, N, P, R, S, V, W or Y
  VARIANT 50: note = A, D, G, I, K, N, P, R, S, V, W or Y
  VARIANT 76..84: note = G, A, V, I, P, D, R, K, Y, W, N or S
ATGVRAXXGNENXLEIEELARFAVDEHNKKENALLEFVRVVKAKEQVVAXTMYYLTLEAKDGGKKKLYEAKVWVKXXXXXXXXXNFKELQEFKPVGDA

SEQ ID NO: 5 - protein, 58 aa, synthetic construct
  VARIANT 9..10: note = M, A, R, K, L, E, Q, W, Y, H, V or S
  VARIANT 11: note = M, A, R, K, L, E, Q, W, Y, H or V
  VARIANT 13..14: note = M, A, R, K, L, E, Q, W, Y, H, V or S
  VARIANT 17: note = M, A, R, K, L, E, Q, W, Y, H or V
  VARIANT 18: note = M, A, R, K, L, E, Q, W, Y, H or S
  VARIANT 24..25: note = M, A, R, K, L, E, Q, W, Y, H or S
  VARIANT 27: note = M, A, R, K, L, E, Q, W, Y, H, V or S
  VARIANT 28: note = M, A, R, K, L, E, Q, W, Y, H or V
  VARIANT 32: note = M, A, R, K, L, E, Q, W, Y, H, V or S
  VARIANT 35: note = M, A, R, K, L, E, Q, W, Y, H, V or S
  VARIANT 39: note = S or R
  VARIANT 40: note = Q or R
  VARIANT 41: note = S, H or P
VDNKFNKEXXXAXXEIXXLPNLNXXQXXAFIXSLXDDPXXXANLLAEAKKLNDAQAPK

SEQ ID NO: 6 - protein, 37 aa, synthetic construct
TCSQDEFRCHDGKCISRQFVCDSDRDCLDGSDEASCP

SEQ ID NO: 7 - protein, 37 aa, synthetic construct
TCKSGDFSCGGRVNRCIPQFWRCDGQVDCDNGSDEQG

SEQ ID NO: 8 - protein, 92 aa, synthetic construct
ATGVRAVPGNENSLEIEELARFAVDEHNKKENALLEFVRVVKAKEQVVAGTMYYLTLEAKDGGKKKLYEAKVWVKPWENFKELQEFKPVGDA

SEQ ID NO: 9 - protein, 58 aa, synthetic construct [sequence omitted]

SEQ ID NO: 10 - protein, 41 aa, synthetic construct
MCDIPPFVCWWPSWCVWATWVCDGAPDCIDNYDEWICTPTP

SEQ ID NO: 11 - protein, 41 aa, synthetic construct
MCWVKIFRCNIVGVCIVNTWVCDGRVDCSDPPDEGWCTPTP

SEQ ID NO: 12 - protein, 41 aa, synthetic construct
MCAIDNFPCNWYIDCIGATWVCDGIIDCIDIADEYDCTPTP

SEQ ID NO: 13 - protein, 59 aa, synthetic construct
MVDNKFNKEQWLAWYEIHYLPNLNAMQHRAFILSPYDDPSQSANLLAEAKKLNDAQAPK

SEQ ID NO: 14 - protein, 59 aa, synthetic construct
MVDNKFNKEWYAAELEIWELPNLNMKQWYAFIYSLEDDPSQSANLLAEAKKLNDAQAPK

SEQ ID NO: 15 - protein, 59 aa, synthetic construct
MVDNKFNKEYSVAWLEIYLLPNLNMMQLRAFIVSLEDDPSQSANLLAEAKKLNDAQAPK

SEQ ID NO: 16 - protein, 59 aa, synthetic construct
MVDNKFNKEELMAMYEIEYLPNLNAYQMVAFIVSLVDDPSQSANLLAEAKKLNDAQAPK

SEQ ID NO: 17 - protein, 59 aa, synthetic construct
MVDNKFNKELAYAHMEIWYLPNLNSQQWYAFIESLRDDPSQSANLLAEAKKLNDAQAPK

SEQ ID NO: 18 - protein, 58 aa, synthetic construct
MVDNKFNKEMSYAVLEILRLPNLNLMQLAFIYSLWDDPSQSANLLAEAKKLNDAQAPK

SEQ ID NO: 19 - protein, 59 aa, synthetic construct
MVDNKFNKEYYYAAVEIQYLPNLNMWQMLAFIVSLYDDPRRPANLLAEAKKLNDAQAPK

SEQ ID NO: 20 - protein, 59 aa, synthetic construct
MVDNKFNKEMYVAVLEIHSLPNLNLMQLVAFILSLYDDPSQSANLLAEAKKLNDAQAPK

SEQ ID NO: 21 - protein, 59 aa, synthetic construct
MVDNKFNKEWSWAYSEIHLLPNLNHLQHYAFIMSLQDDPRRHANLLAEAKKLNDAQAPK

SEQ ID NO: 22 - protein, 59 aa, synthetic construct
MVDNKFNKEYMLAELEIAALPNLNHWQEVAFIVSLVDDPSQSANLLAEAKKLNDAQAPK

SEQ ID NO: 23 - protein, 59 aa, synthetic construct
MVDNKFNKESQYAYAEIYLLPNLNHWQSVAFIWSLYDDPSQSANLLAEAKKLNDAQAPK

SEQ ID NO: 24 - protein, 59 aa, synthetic construct
MVDNKFNKEKVHAMLEIRWLPNLNMLQAYAFIQSLHDDPSQSANLLAEAKKLNDAQAPK

SEQ ID NO: 25 - protein, 59 aa, synthetic construct
MVDNKFNKEERAAYVEIHWLPNLNSKQWEAFIMSLQDDPSQSANLLAEAKKLNDAQAPK

SEQ ID NO: 26 - protein, 99 aa, synthetic construct
MATGVRAVPGNENSLEIEELARFAVDEHNKKENALLEFVRVVKAKEQVVAGTMYYLTLEAKDGGKKKLYEAKVWVKIRAIVYWSGNFKELQEFKPVGDA

SEQ ID NO: 27 - protein, 99 aa, synthetic construct
MATGVRAVPGNENSLEIEELARFAVDEHNKKENALLEFVRVMKAKEQVVAGTMYYLTLEAKDGGKKKLYEAKVWVKGVIYAYSYSNFKELQEFKPVGDA

SEQ ID NO: 28 - protein, 99 aa, synthetic construct
MATGVRAVPGNENSLEIEELARFAVDEHNKKENALLEFVRVVKAKEQVVAGTMYYLTLEAKDGGKKKLYEAKVWVKAGYIGRSKANFKELQEFKPVGDA

SEQ ID NO: 29 - protein, 99 aa, synthetic construct
MATGVRAVPGNENSLEIEELARFAVDEHNKKENALLEFVRVVKAKEQVVAGTMYYLTLEAKDGGKKKLYEAKVWVKAAGYYVYAPNFKELQEFKPVGDA

SEQ ID NO: 30 - protein, 99 aa, synthetic construct
MATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDA

SEQ ID NO: 31 - protein, 99 aa, synthetic construct
MATGVRAVPGNENSLEIEELARFAVDEHNKKENALLEFVRVVKAKEQVVAGTMYYLTLEAKDGGKKKLYEAKVWVKISRPGWWPWNFKELQEFKPVGDA

SEQ ID NO: 32 - protein, 99 aa, synthetic construct
MATGVRAVPGNENSLEIEELARFAVDEHNKKENALLEFVRVVKAKEQVVAGTMYYLTLEAKDGGKKKLYEAKVWVKSRSVRIWYSNFKELQEFKPVGDA

SEQ ID NO: 33 - protein, 99 aa, synthetic construct
MATGVRAVPGNENSLEIEELARFAVDEHNKKENALLEFVRVVKAKEQVVAGTMYYLTLEAKDGGKKKLYEAKVWVKVRRDIVVPRNFKELQEFKPVGDA

SEQ ID NO: 34 - protein, 99 aa, synthetic construct
MATGVRAVPGNENSLEIEELARFAVDEHNKKENALLEFVRVVKAKEQVVACTMYYLTLEAKDDGKKKLYEAKVWVKSRVRVIRWGNFKELQEFKPVGDA

SEQ ID NO: 35 - protein, 99 aa, synthetic construct
MATGVRAVPGNENSLEIEELARFAVDEHNKKENALLEFVRVVKAKEQVVAGTMYYLTLEAKDGGKKKLYEAKVWVKVRDIVSIRGNFKELQEFKPVGDA

SEQ ID NO: 36 - protein, 99 aa, synthetic construct
MATGVRAVPSNENSLEIEELARFAVDEHNKKENALLEFVRVVKAKEQVVAGTMYYLTLEAKDGGKKKLYEAKVWVKAIKYVRYKANFKELQEFKPVGDA

SEQ ID NO: 37 - protein, 99 aa, synthetic construct
MATGVRAVSGNENSLEIEELARFAVDEHNKKENALLEFVRVVKAKEQVVAGTMYYLTLEAKDGGKKKLYEAKVWVKSPVYWPKGGNFKELQEFKPVGDA

SEQ ID NO: 38 - protein, 99 aa, synthetic construct
MATGVRAVPGNENSLEIEELARFAVDEHNKKENALLEFVRVVKAKEQVVAGTMYYLTLEAKDGGKKKLYEAKVWVKSASYYVYSYNFKELQEFKPVGDA

SEQ ID NO: 39 - protein, 99 aa, synthetic construct
MATGVRAVPGNENSLEIEELARFAVDEHNKKENALLEFVRVVKAKEQVVAGTMYYLTLEAKDGGKKKLYEAKVWVKWRAVRWWWGNFKELQEFKPVGDA

SEQ ID NO: 40 - protein, 99 aa, synthetic construct
MATGVRAVPGNENSLEIEELARFAVDEHNKKENALLEFVRVVKAKEQVVAGTMYYLTLEAKDGGKKKLYEAKVWVKVVGKWWVNINFKELQEFKPVGDA

SEQ ID NO: 41 - protein, 99 aa, synthetic construct
MATGVRAVPGNENSLEIEELARFAVDEHNKKENALLEFVRVVKAKEQVVAGTMYYLTLEAKDGGKKKLYEAKVWVKARYVVYYRWNFKELQEFKPVGDA

SEQ ID NO: 42 - protein, 99 aa, synthetic construct
MATGVRAVPGNENSLEIEELARFAVDEHNKKENALLEFVRVVKAKEQVVAGTMYYLTLEAKDGGKKKLYEAKVWVKGRYSVYWYVNFKELQEFKPVGDA

SEQ ID NO: 43 - protein, 99 aa, synthetic construct
MATGVRAVPGNENSLEIEELARFAVDEHNKKENALLEFVRVVKAKEQVVAGTMYYLTLEAKDGGKKKLYEAKVWVKVRYPPNIWKNFKELQEFKPVGDA

SEQ ID NO: 44 - protein, 104 aa, synthetic construct
MATGVRAVPGNENSLEIEELARFAVDEHNKKENALLEFVRMVKAKEQYISVDKWVATMYYLTLEAKDGGKKKLYEAKVWVKYGGSCWRGGNFKELQEFKPVGDA

SEQ ID NO: 45 - protein, 99 aa, synthetic construct
MATGVRAVPGNENSLEIEELARFAVDEHNKKENALLEFVRVVKAKEQVVAGTMYYLTLEAKDGGKKKLYEAKVWVKVAAYPRASINFKELQEFKPVGDA

SEQ ID NO: 46 - protein, 99 aa, synthetic construct
MATGVRAVPGNENSLEIEELARFAVDEHNKKENALLEFVRVVKAKEQVVAGTMYYLTLEAKDGGKKKLYEAKVWVKIGIPIRIWSNFKELQEFKPVGDA

SEQ ID NO: 47 - protein, 99 aa, synthetic construct
MATGVRAVPGNENSLEIEELARFAVDEHNKKENALLEFVRVVKAKEQVVAGTMYYLTLEAKDGGKKKLYEAKVWVKVNIYVISPRNFKELQEFKPVGDA

SEQ ID NO: 48 - protein, 99 aa, synthetic construct
MATGVRAVPGNENSLEIEELARFAVDEHNKKENALLEFVCVVKAKEQVVAGTMYYLTLEAKDGGKKKLYEAKVWVKVAPIYSVDSNFKELQEFKPVGDA

SEQ ID NO: 49 - protein, 99 aa, synthetic construct
MATGVRAVPGNENSLEIEELARFAVDEHNKKENALLEFVRVVKAKEQVVAGTMYYLTLEAKDGGKKKLYEAKVWVKSVAIRNWYWNFKELQEFKPVGDA

SEQ ID NO: 50 - protein, 99 aa, synthetic construct
MATGVRAVPGNENSLEIEELARFAVDEHNKKENALLEFVRVVKAKEQVVAGTMYYLTLEAKDGGKKKLYEAKVWVKVRRYSPPNRNFKELQEFKPVGDA

SEQ ID NO: 51 - protein, 99 aa, synthetic construct
MATGVRAVPGNENSLEIEELARFAVDEHNKKENALLEFVRVVKAKEQVVAGTMYYLTLEAKDGGKKKLYEAKVWVKVRYWVDRRKNFKELQEFKPVGDA

SEQ ID NO: 52 - protein, 99 aa, synthetic construct
MATGVRAMPGNENSLEIEELARFAVDEHNKKENALLEFVRVVKAKEQVVAGTMYYLTLEAKDGGKKKLYEAKVWVKVWWVVSDPANFKELQEFKPVGDA

SEQ ID NO: 53 - protein, 104 aa, synthetic construct
MATGVRAVPGNENSLEIEELARFAVDEHNKKENALLEFVRVVKAKEQRARYYYVNRTMYYLTLEAKDGGKKKLYEAKVWVKWVIRGDSPANFKELQEFKLVGDA

SEQ ID NO: 54 - protein, 40 aa, synthetic construct; identical to SEQ ID NO: 1

SEQ ID NO: 55 - protein, 40 aa, synthetic construct
CDIPPFVCWWPSWCVWATWVCDGAPDCIDNYDEWICTPTP

SEQ ID NO: 56 - protein, 40 aa, synthetic construct
CWVKIFRCNIVGVCIVNTWVCDGRVDCSDPPDEGWCTPTP

SEQ ID NO: 57 - protein, 40 aa, synthetic construct
CAIDNFPCNWYIDCIGATWVCDGIIDCIDIADEYDCTPTP

SEQ ID NO: 58 - protein, 98 aa, synthetic construct; identical to SEQ ID NO: 2

SEQ ID NO: 59 - protein, 98 aa, synthetic construct
ATGVRAVPGNENSLEIEELARFAVDEHNKKENALLEFVRVVKAKEQVVAGTMYYLTLEAKDGGKKKLYEAKVWVKIRAIVYWSGNFKELQEFKPVGDA

SEQ ID NO: 60 - protein, 98 aa, synthetic construct
ATGVRAVPGNENSLEIEELARFAVDEHNKKENALLEFVRVMKAKEQVVAGTMYYLTLEAKDGGKKKLYEAKVWVKGVIYAYSYSNFKELQEFKPVGDA

SEQ ID NO: 61 - protein, 98 aa, synthetic construct
ATGVRAVPGNENSLEIEELARFAVDEHNKKENALLEFVRVVKAKEQVVAGTMYYLTLEAKDGGKKKLYEAKVWVKAGYIGRSKANFKELQEFKPVGDA

SEQ ID NO: 62 - protein, 98 aa, synthetic construct
ATGVRAVPGNENSLEIEELARFAVDEHNKKENALLEFVRVVKAKEQVVAGTMYYLTLEAKDGGKKKLYEAKVWVKAAGYYVYAPNFKELQEFKPVGDA

SEQ ID NO: 63 - protein, 98 aa, synthetic construct
ATGVRAVPGNENSLEIEELARFAVDEHNKKENALLEFVRVVKAKEQVVAGTMYYLTLEAKDGGKKKLYEAKVWVKISRPGWWPWNFKELQEFKPVGDA

SEQ ID NO: 64 - protein, 98 aa, synthetic construct
ATGVRAVPGNENSLEIEELARFAVDEHNKKENALLEFVRVVKAKEQVVAGTMYYLTLEAKDGGKKKLYEAKVWVKSRSVRIWYSNFKELQEFKPVGDA

SEQ ID NO: 65 - protein, 98 aa, synthetic construct
ATGVRAVPGNENSLEIEELARFAVDEHNKKENALLEFVRVVKAKEQVVAGTMYYLTLEAKDGGKKKLYEAKVWVKVRRDIVVPRNFKELQEFKPVGDA

SEQ ID NO: 66 - protein, 98 aa, synthetic construct
ATGVRAVPGNENSLEIEELARFAVDEHNKKENALLEFVRVVKAKEQVVAGTMYYLTLEAKDGGKKKLYEAKVWVKVRDIVSIRGNFKELQEFKPVGDA

SEQ ID NO: 67 - protein, 98 aa, synthetic construct
ATGVRAVPSNENSLEIEELARFAVDEHNKKENALLEFVRVVKAKEQVVAGTMYYLTLEAKDGGKKKLYEAKVWVKAIKYVRYKANFKELQEFKPVGDA

SEQ ID NO: 68 - protein, 98 aa, synthetic construct
ATGVRAVPGNENSLEIEELARFAVDEHNKKENALLEFVRVVKAKEQVVAGTMYYLTLEAKDGGKKKLYEAKVWVKSASYYVYSYNFKELQEFKPVGDA

SEQ ID NO: 69 - protein, 98 aa, synthetic construct
ATGVRAVPGNENSLEIEELARFAVDEHNKKENALLEFVRVVKAKEQVVAGTMYYLTLEAKDGGKKKLYEAKVWVKWRAVRWWWGNFKELQEFKPVGDA

SEQ ID NO: 70 - protein, 98 aa, synthetic construct
ATGVRAVPGNENSLEIEELARFAVDEHNKKENALLEFVRVVKAKEQVVAGTMYYLTLEAKDGGKKKLYEAKVWVKVVGKWWVNINFKELQEFKPVGDA

SEQ ID NO: 71 - protein, 98 aa, synthetic construct
ATGVRAVPGNENSLEIEELARFAVDEHNKKENALLEFVRVVKAKEQVVAGTMYYLTLEAKDGGKKKLYEAKVWVKARYVVYYRWNFKELQEFKPVGDA

SEQ ID NO: 72 - protein, 98 aa, synthetic construct
ATGVRAVPGNENSLEIEELARFAVDEHNKKENALLEFVRVVKAKEQVVAGTMYYLTLEAKDGGKKKLYEAKVWVKGRYSVYWYVNFKELQEFKPVGDA

SEQ ID NO: 73 - protein, 98 aa, synthetic construct
ATGVRAVPGNENSLEIEELARFAVDEHNKKENALLEFVRVVKAKEQVVAGTMYYLTLEAKDGGKKKLYEAKVWVKVRYPPNIWKNFKELQEFKPVGDA

SEQ ID NO: 74 - protein, 98 aa, synthetic construct
ATGVRAVPGNENSLEIEELARFAVDEHNKKENALLEFVRVVKAKEQVVAGTMYYLTLEAKDGGKKKLYEAKVWVKVAAYPRASINFKELQEFKPVGDA

SEQ ID NO: 75 - protein, 98 aa, synthetic construct
ATGVRAVPGNENSLEIEELARFAVDEHNKKENALLEFVRVVKAKEQVVAGTMYYLTLEAKDGGKKKLYEAKVWVKIGIPIRIWSNFKELQEFKPVGDA

SEQ ID NO: 76 - protein, 98 aa, synthetic construct
ATGVRAVPGNENSLEIEELARFAVDEHNKKENALLEFVRVVKAKEQVVAGTMYYLTLEAKDGGKKKLYEAKVWVKVNIYVISPRNFKELQEFKPVGDA

SEQ ID NO: 77 - protein, 98 aa, synthetic construct
ATGVRAVPGNENSLEIEELARFAVDEHNKKENALLEFVCVVKAKEQVVAGTMYYLTLEAKDGGKKKLYEAKVWVKVAPIYSVDSNFKELQEFKPVGDA

SEQ ID NO: 78 - protein, 98 aa, synthetic construct
ATGVRAVPGNENSLEIEELARFAVDEHNKKENALLEFVRVVKAKEQVVAGTMYYLTLEAKDGGKKKLYEAKVWVKSVAIRNWYWNFKELQEFKPVGDA

SEQ ID NO: 79 - protein, 98 aa, synthetic construct
ATGVRAVPGNENSLEIEELARFAVDEHNKKENALLEFVRVVKAKEQVVAGTMYYLTLEAKDGGKKKLYEAKVWVKVRRYSPPNRNFKELQEFKPVGDA

SEQ ID NO: 80 - protein, 99 aa, synthetic construct
AATGVRAVPGNENSLEIEELARFAVDEHNKKENALLEFVRVVKAKEQVVAGTMYYLTLEAKDGGKKKLYEAKVWVKVRYWVDRRKNFKELQEFKPVGDA

SEQ ID NO: 81 - protein, 103 aa, synthetic construct; identical to SEQ ID NO: 3

SEQ ID NO: 82 - protein, 103 aa, synthetic construct
ATGVRAVPGNENSLEIEELARFAVDEHNKKENALLEFVRMVKAKEQYISVDKWVATMYYLTLEAKDGGKKKLYEAKVWVKYGGSCWRGGNFKELQEFKPVGDA

SEQ ID NO: 83 - protein, 103 aa, synthetic construct
ATGVRAVPGNENSLEIEELARFAVDEHNKKENALLEFVRVVKAKEQRARYYYVNRTMYYLTLEAKDGGKKKLYEAKVWVKWVIRGDSPANFKELQEFKLVGDA

SEQ ID NO: 84 - protein, 98 aa, synthetic construct; identical to SEQ ID NO: 4

SEQ ID NO: 85 - protein, 98 aa, synthetic construct
ATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDA

SEQ ID NO: 86 - protein, 98 aa, synthetic construct
ATGVRAVPGNENSLEIEELARFAVDEHNKKENALLEFVRVVKAKEQVVACTMYYLTLEAKDDGKKKLYEAKVWVKSRVRVIRWGNFKELQEFKPVGDA

SEQ ID NO: 87 - protein, 98 aa, synthetic construct
ATGVRAVSGNENSLEIEELARFAVDEHNKKENALLEFVRVVKAKEQVVAGTMYYLTLEAKDGGKKKLYEAKVWVKSPVYWPKGGNFKELQEFKPVGDA

SEQ ID NO: 88 - protein, 98 aa, synthetic construct
ATGVRAMPGNENSLEIEELARFAVDEHNKKENALLEFVRVVKAKEQVVAGTMYYLTLEAKDGGKKKLYEAKVWVKVWWVVSDPANFKELQEFKPVGDA

SEQ ID NO: 89 - protein, 103 aa, synthetic construct
ATGVRAXXGNENXLEIEELARFAVDEHNKKENALLEFVRVVKAKEQXXXXXXXXXTMYYLTLEAKDGGKKKLYEAKVWVKXXXXXXXXXNFKELQEFKPVGDA

SEQ ID NO: 90 - protein, 58 aa, synthetic construct; identical to SEQ ID NO: 5

SEQ ID NO: 91 - protein, 58 aa, synthetic construct
VDNKFNKEQWLAWYEIHYLPNLNAMQHRAFILSPYDDPSQSANLLAEAKKLNDAQAPK

SEQ ID NO: 92 - protein, 58 aa, synthetic construct
VDNKFNKEWYAAELEIWELPNLNMKQWYAFIYSLEDDPSQSANLLAEAKKLNDAQAPK

SEQ ID NO: 93 - protein, 58 aa, synthetic construct
VDNKFNKEYSVAWLEIYLLPNLNMMQLRAFIVSLEDDPSQSANLLAEAKKLNDAQAPK

SEQ ID NO: 94 - protein, 58 aa, synthetic construct
VDNKFNKEELMAMYEIEYLPNLNAYQMVAFIVSLVDDPSQSANLLAEAKKLNDAQAPK

SEQ ID NO: 95 - protein, 58 aa, synthetic construct
VDNKFNKELAYAHMEIWYLPNLNSQQWYAFIESLRDDPSQSANLLAEAKKLNDAQAPK

SEQ ID NO: 96 - protein, 57 aa, synthetic construct
VDNKFNKEMSYAVLEILRLPNLNLMQLAFIYSLWDDPSQSANLLAEAKKLNDAQAPK

SEQ ID NO: 97 - protein, 58 aa, synthetic construct
VDNKFNKEYYYAAVEIQYLPNLNMWQMLAFIVSLYDDPRRPANLLAEAKKLNDAQAPK

SEQ ID NO: 98 - protein, 58 aa, synthetic construct
VDNKFNKEMYVAVLEIHSLPNLNLMQLVAFILSLYDDPSQSANLLAEAKKLNDAQAPK

SEQ ID NO: 99 - protein, 58 aa, synthetic construct
VDNKFNKEWSWAYSEIHLLPNLNHLQHYAFIMSLQDDPRRHANLLAEAKKLNDAQAPK

SEQ ID NO: 100 - protein, 58 aa, synthetic construct
VDNKFNKEYMLAELEIAALPNLNHWQEVAFIVSLVDDPSQSANLLAEAKKLNDAQAPK

SEQ ID NO: 101 - protein, 58 aa, synthetic construct
VDNKFNKESQYAYAEIYLLPNLNHWQSVAFIWSLYDDPSQSANLLAEAKKLNDAQAPK

SEQ ID NO: 102 - protein, 58 aa, synthetic construct
VDNKFNKEKVHAMLEIRWLPNLNMLQAYAFIQSLHDDPSQSANLLAEAKKLNDAQAPK

SEQ ID NO: 103 - protein, 58 aa, synthetic construct
VDNKFNKEERAAYVEIHWLPNLNSKQWEAFIMSLQDDPSQSANLLAEAKKLNDAQAPK

SEQ ID NO: 104 - protein, 98 aa, synthetic construct
ATGVRAXXXNENXLEIEELARFAVDEHNKKENALLEFVXVXKAKEQVVAXTMYYLTLEAKDXGKKKLYEAKVWVKXXXXXXXXXNFXELQEFKPVGDA

SEQ ID NO: 105 - protein, 103 aa, synthetic construct
ATGVRAVPGNENSLEIEELARFAVDEHNKKENALLEFVRXVKAKEQXXXXXXXXXTMYYLTLEAKDGGKKKLYEAKVWVKXXXXXXXXXNFKELQEFKXVGDA

SEQ ID NO: 106 - protein, 103 aa, synthetic construct
ATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDA

SEQ ID NO: 107 - protein, 103 aa, synthetic construct
ATGVRAXXXNENXLEIEELARFAVDEHNKKENALLEFVXXXKAKEQXXXXXXXXXTMYYLTLEAKDGGKKKLYEAKVWVKXXXXXXXXXNFXELQEFKXVGDA

SEQ ID NO: 108 - protein, 57 aa, synthetic construct
VDNKFNKEXXXXXEIXXLPNLNXXQXXAFIXSXXDDPXXXANLLAEAKKLNDAQAPK

SEQ ID NO: 109 - protein, 18 aa, synthetic construct
GGGGSGGSGGGGSGGRGS

SEQ ID NO: 110 - protein, 5 aa, synthetic construct
AAAAA

SEQ ID NO: 111 - protein, 10 aa, synthetic construct
AAAAAAAAAA

SEQ ID NO: 112 - protein, 11 aa, synthetic construct
AAAAAAGGGGG

SEQ ID NO: 113 - protein, 5 aa, synthetic construct
AGAGA

SEQ ID NO: 114 - protein, 5 aa, synthetic construct
EAAAK

SEQ ID NO: 115 - protein, 10 aa, synthetic construct
EAAAKEAAAK

SEQ ID NO: 116 - protein, 6 aa, synthetic construct
EKAREK

SEQ ID NO: 117 - protein, 5 aa, synthetic construct
GAAGA

SEQ ID NO: 118 - protein, 6 aa, synthetic construct
GASGAS

SEQ ID NO: 119 - protein, 5 aa, synthetic construct
GEGEF

SEQ ID NO: 120 - protein, 5 aa, synthetic construct
GGGGS

SEQ ID NO: 121 - protein, 9 aa, synthetic construct
GGGGSGGGG

SEQ ID NO: 122 - protein, 10 aa, synthetic construct
GGGGSGGGGS

SEQ ID NO: 123 - protein, 14 aa, synthetic construct
GGGGSGGGGSGGGG

SEQ ID NO: 124 - protein, 15 aa, synthetic construct
GGGGSGGGGSGGGGS

SEQ ID NO: 125 - protein, 20 aa, synthetic construct
GGGGSGGGGSGGGGSGGGGS

SEQ ID NO: 126 - protein, 25 aa, synthetic construct
GGGGSGGGGSGGGGSGGGGSGGGGS

SEQ ID NO: 127 - protein, 4 aa, synthetic construct
GGGS

SEQ ID NO: 128 - protein, 8 aa, synthetic construct
GGGSGGGS

SEQ ID NO: 129 - protein, 11 aa, synthetic construct
GGGSGGGSGGG

SEQ ID NO: 130 - protein, 12 aa, synthetic construct
GGGSGGGSGGGS

SEQ ID NO: 131 - protein, 5 aa, synthetic construct
GGSGG

SEQ ID NO: 132 - protein, 6 aa, synthetic construct
GGSGGS

SEQ ID NO: 133 - protein, 9 aa, synthetic construct
GGSGGSGGS

SEQ ID NO: 134 - protein, 12 aa, synthetic construct
GGSGGSGGSGGS

SEQ ID NO: 135 - protein, 6 aa, synthetic construct
GPGGPG

SEQ ID NO: 136 - protein, 5 aa, synthetic construct
GPGPG

SEQ ID NO: 137 - protein, 6 aa, synthetic construct
GPGPGP

SEQ ID NO: 138 - protein, 6 aa, synthetic construct
GPPGPP

SEQ ID NO: 139 - protein, 6 aa, synthetic construct
GPSGPG

SEQ ID NO: 140 - protein, 6 aa, synthetic construct
GPSGPS

SEQ ID NO: 141 - protein, 8 aa, synthetic construct
GPSGSGPG

SEQ ID NO: 142 - protein, 19 aa, synthetic construct
GSGGGGSGGGGSGGGGSGS

SEQ ID NO: 143 - protein, 14 aa, synthetic construct
GSGGGGSGGGGSGS

SEQ ID NO: 144 - protein, 9 aa, synthetic construct
GSGGGGSGS

SEQ ID NO: 145 - protein, 16 aa, synthetic construct
GSGGGSGGGSGGGSGS

SEQ ID NO: 146 - protein, 12 aa, synthetic construct
GSGGGSGGGSGS

SEQ ID NO: 147 - protein, 8 aa, synthetic construct
GSGGGSGS

SEQ ID NO: 148 - protein, 6 aa, synthetic construct
GSGGSG

SEQ ID NO: 149 - protein, 4 aa, synthetic construct
GSGS

SEQ ID NO: 150 - protein, 10 aa, synthetic construct
GSGSAGSGSA

SEQ ID NO: 151 - protein, 6 aa, synthetic construct
GSGSGS

SEQ ID NO: 152 - protein, 9 aa, synthetic construct
GSGSGSGSG

SEQ ID NO: 153 - protein, 4 aa, synthetic construct
GSSG

SEQ ID NO: 154 - protein, 8 aa, synthetic construct
GSSGGGGS

SEQ ID NO: 155 - protein, 10 aa, synthetic construct
KKKKKGGGGG

SEQ ID NO: 156 - protein, 5 aa, synthetic construct
PAPAP

SEQ ID NO: 157 - protein, 9 aa, synthetic construct
PGPGPGPGP

SEQ ID NO: 158 - protein, 6 aa, synthetic construct
QPGSGP

SEQ ID NO: 159 - protein, 8 aa, synthetic construct
SGAGSAGA

SEQ ID NO: 160 - protein, 6 aa, synthetic construct
SGGSGG

SEQ ID NO: 161 - protein, 9 aa, synthetic construct
SGGSGGSGG

SEQ ID NO: 162 - protein, 12 aa, synthetic construct
SGGSGGSGGSGG

SEQ ID NO: 163 - protein, 14 aa, synthetic construct
SGSGGSGGSGGSGG

SEQ ID NO: 164 - protein, 8 aa, synthetic construct
SGSGSGSG

SEQ ID NO: 165 - protein, 8 aa, synthetic construct
SSGSSGSS

SEQ ID NO: 166 - protein, 11 aa, synthetic construct
TGTGGSGSGTG

SEQ ID NO: 167 - protein, 10 aa, synthetic construct
TGTGGTGTGG

SEQ ID NO: 168 - protein, 14 aa, synthetic construct
YPKYVKQNTLKLAT

SEQ ID NO: 169 - protein, 118 aa, synthetic construct
GGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGY

SEQ ID NO: 170 - protein, 116 aa, synthetic construct
GGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDA

SEQ ID NO: 171 - protein, 349 aa, synthetic construct
GGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAG

SEQ ID NO: 172 - protein, 350 aa, synthetic construct
GGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGY

SEQ ID NO: 173 - protein, 348 aa, synthetic construct
GGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDA

SEQ ID NO: 174 - protein, 466 aa, synthetic construct
GGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGY

SEQ ID NO: 175 - protein, 464 aa, synthetic construct
GGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDA

SEQ ID NO: 176 - protein, 582 aa, synthetic construct
GGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGY

SEQ ID NO: 177 - protein, 580 aa, synthetic construct
GGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDA

SEQ ID NO: 178 - protein, 698 aa, synthetic construct
GGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGY

SEQ ID NO: 179 - protein, 696 aa, synthetic construct
GGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDA

SEQ ID NO: 180 - protein, 123 aa, synthetic construct
GGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGY

SEQ ID NO: 181 - protein, 121 aa, synthetic construct
GGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDA

SEQ ID NO: 182 - protein, 365 aa, synthetic construct
GGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGY

SEQ ID NO: 183 - protein, 363 aa, synthetic construct
GGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDA

SEQ ID NO: 184 - protein, 486 aa, synthetic construct
GGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGY

SEQ ID NO: 185 - protein, 484 aa, synthetic construct
GGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDA

SEQ ID NO: 186 - protein, 486 aa, synthetic construct; identical to SEQ ID NO: 184

SEQ ID NO: 187 - protein, 484 aa, synthetic construct; identical to SEQ ID NO: 185

SEQ ID NO: 188 - protein, 728 aa, synthetic construct
GGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGY

SEQ ID NO: 189 - protein, 726 aa, synthetic construct
GGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDA

SEQ ID NO: 190 - protein, 234 aa, synthetic construct
GGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGY

SEQ ID NO: 191 - protein, 232 aa, synthetic construct
GGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDAGGGGSGGSGGGGSGGRGSATGVRAVSGNENGLEIEELARFAVDEHNKKENALLEFVRVVKAKEQVVAGTMYYLTLEAKDGGKKKLYEAKVWVKRIVRVAVWVNFRELQEFKPVGDA

SEQ ID NO: 192 - protein, 244 aa, synthetic construct
GGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGY

SEQ ID NO: 193 - protein, 242 aa, synthetic construct
GGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDAGGGGSGGSGGGGSGGGGSATGVRAVPGNENSLEIEELARFAVDEHNKKENALLEFVRVVKAKEQSRYPKGNKVTMYYLTLEAKDGGKKKLYEAKVWVKVAVVARAAVNFKELQEFKPVGDA

SEQ ID NO: 194 - DNA, 354 bp, synthetic construct
  source 1..354: mol_type = other DNA
gtagcccgcgtctcctaccggtttaaattcttgtaattcacggaagttgacccagacggcgacgcgtacaatacgctttacccagaccttggcttcgtataattttttcttaccaccatcctttgcttctaatgtaaggtagtacattgtgcctgcgacaacctgttctttcgcttttaccacacgtacgaattccaacagcgcgttttctttcttattatgctcgtcgacggcaaaacgggccagttcctcaatctccaggccgttttcattaccggatactgcacggaccccagtcgctgagcctcttccacctgatcctccacctcctgacccaccgctccccccgccgcc

SEQ ID NO: 195 - DNA, 369 bp, synthetic construct
  source 1..369: mol_type = other DNA
gtagcccgcgtccccaacgggcttgaactcttgaagttctttgaaattgacggcagcgcgcgcaacgactgctaccttaacccaaactttggcttcgtaaagtttctttttacccccgtctttggcctccaatgtcagataatacatggtaactttattacccttggggtaacggctttgttctttcgccttgacgacgcgtacgaactcaagaagtgcgttctcttttttattatgttcgtcgacggcaaaacgagccaactcttcgatttcaagcgaattttcattaccgggcaccgcgcgcaccccggttgcgctgccaccacctccactgcctccaccgccagagcctccacttcctcccccgcc

(19) United States
(12) Patent Application Publication
(10) Pub. No.: US 2023/0391832 A1
(43) Pub. Date: Dec. 7, 2023

(54) COMPOSITIONS COMPRISING AAV-BINDING POLYPEPTIDES AND METHODS OF USING THE SAME

(71) Applicant: Isolere Bio, Inc., Durham, NC (US)

(72) Inventors: Kelli M. LUGINBUHL, Durham, NC (US); Linus HYUN, Durham, NC (US); Michael Dzuricky, Durham, NC (US)

(21) Appl. No.: 18/309,154

(22) Filed: Apr. 28, 2023

Related U.S. Application Data
(60) Provisional application No. 63/336,633, filed on Apr. 29, 2022.

(51) Int. Cl. C07K 14/005

(52) U.S. Cl. CPC C07K 14/005; C12N 2750/14122

(57) ABSTRACT

Disclosed herein are AAV binding proteins and methods of making and using the same.

### CROSS-REFERENCE TO RELATED APPLICATIONS

This application claims priority to U.S. Provisional No. 63/336,633, filed Apr. 29, 2022. This application is incorporated by reference herein in its entirety.

### REFERENCE TO AN ELECTRONIC SEQUENCE LISTING

The contents of the electronic sequence listing (ISOL_009_01US_SeqList_ST26.xml; Size: 199,483 bytes; and Date of Creation: Apr. 27, 2023) are herein incorporated by reference in its entirety.

### FIELD

The present disclosure is generally related to compositions and methods for purification of biologics. More specifically, the disclosure is related to purification matrices comprising adeno-associated virus-binding polypeptides and methods of using the same.

### BACKGROUND OF THE INVENTION

Adeno-associated virus (AAV) is a promising vehicle for delivery of one or more therapeutic genes in gene therapy. AAV is a small, replication-deficient DNA virus that is capable of integrating into the genome of an infected cell. AAV facilitates persistent expression of the therapeutic gene and reduces the need for repeated dosing of a gene therapy vector.

There are limitations to AAV manufacturing methods, however, that make widespread use of AAVs for gene therapy difficult. Manufacturing of AAV vectors produces three types of capsids: empty, partial, and full. Li et al. Drug Discovery and Development Sciex. 2020. Full capsids contain all of the desired genomic material. In contrast, empty capsids do not contain any genomic material, and partial capsids only contain fragments of the desired genomic material. Full capsids are desirable, and the presence of empty and partial capsids within a pharmaceutical composition may negatively affect the efficacy and safety of the composition. Selective quantification and purification of full capsids is difficult because the external capsid surface on empty and full capsids is highly similar.

Industry standard methods today for separating full from empty virus particles involve inefficient separation methods that work on the basis of charge (ion exchange chromatography) or density (gradient density ultracentrifugation). (see International Publication No. 2004/113494; U.S. Publication No. 2019/0055523; U.S. Pat. Nos. 11,021,689; and 11,015,174) Analytical methods for quantifying full particles relies on separately measuring full capsids (PCR or 260 nm absorbance) as well as total particles (total capsid ELISA, light scattering, 280 nm absorbance).

Thus, there is a need in the art for novel compositions that can bind to AAV capsids, which will aid in purification, and methods for determining the amount of full and empty capsids in a pharmaceutical composition.

### SUMMARY OF THE INVENTION

The present disclosure solves the need in the art for compositions that bind to AAV capsids and methods that distinguish between full and empty AAV capsids. Provided herein, in embodiments, are compositions comprising polypeptides that bind preferentially to full AAV capsids.

In some aspects, the present disclosure provides a composition comprising a polypeptide comprising a sequence CX₁X₂X₃X₄FX₅CX₆X₇X₈X₉X₁₀CX₁₁X₁₂X₁₃TWVCDGX₁₄X₁₅DCX₁₆DX₁₇X₁₈DEX₁₉X₂₀CTPTP (SEQ ID NO: 54). In embodiments, the present disclosure provides a composition comprising a polypeptide comprising a sequence CX₁X₂X₃X₄FX₅CX₆X₇X₈X₉X₁₀CX₁₁X₁₂X₁₃TWVCDGX₁₄X₁₅DCX₁₆DX₁₇X₁₈DEX₁₉X₂₀CTP TP (SEQ ID NO: 54), wherein each of X₁-X₂₀ are independently an amino acid, provided that at least 1, 2, 3, 4, or 5 amino acids of X₁-X₂₀ do not correspond to the amino acid of a wild-type avimer sequence. In some aspects, each of X₁-X₂₀ are independently D, I, P, V, W, S, A, K, R, N, Y, or G. In some aspects, X₁ is D, W, or A. In some aspects, X₂ is I or V. In some aspects, X₃ is P, K, or D. In some aspects, X₄ is P, I, or N. In some aspects, X₅ is V, R, or P. In some aspects, X₆ is W or N. In some aspects, X₇ is W or I. In some aspects, X₈ is P, V, or Y. In some aspects, X₉ is S, G, or I. In some aspects, X₁₀ is W, V, or D. In some aspects, X₁₁ is V or I. In some aspects, X₁₂ is W, V, or G. In some aspects, X₁₃ is A or N. In some aspects, X₁₄ is A, R, or I. In some aspects, X₁₅ is P, V, or I. In some aspects, X₁₆ is I or S. In some aspects, X₁₇ is N, P, or I. In some aspects, X₁₈ is Y, P, or A. In some aspects, X₁₉ is W, G, or Y. In some aspects, X₂₀ is I, W, or D.

In some aspects, the present disclosure provides a composition comprising a polypeptide comprising the sequence of any one of SEQ ID NOS: 55-57. In some aspects, the present disclosure provides a composition comprising a polypeptide comprising a sequence that is at least 90%, at least 91%, at least 92%, at least 93%, at least 94%, at least 95%, at least 96%, at least 97%, at least 98%, or at least 99% identical to any one of SEQ ID NOS: 55-57.

In some aspects, the present disclosure provides a composition comprising a polypeptide comprising a sequence ATGVRAVPGNENSLEIEELARFAVDEH NKKENALLEFVRVVKAKEQVVAGTMYYLTLEAKDGGKKKLYEAKVWVKU₁U₂U₃U₄U₅U₆U₇U₈U₉NEKELQEFKPVGDA (SEQ ID NO: 58). In some aspects, the present disclosure provides a composition comprising a polypeptide comprising a sequence ATGVRAVPGNENSLEIEELARFAVDEHNKKENALLEFVRVVKAKEQVVAG TMYYLTLEAKDGGKKKLYEAKVWVKU₁U₂U₃U₄U₅U₆U₇U₈U₉NFKELQEFKPVGDA (SEQ ID NO: 58), wherein each of U₁-U₉ are independently an amino acid, provided that at least 1, 2, 3, 4, or 5 amino acids of U₁-U₉ do not correspond to the amino acid of a wild-type affimer sequence. In some aspects, each of U₁-U₉ are independently I, G, A, S, V, W, R, Y, D, K, P, V, or C. In some aspects, U₁ is I, G, A, S, V, W, or A. In some aspects, U₂ is R, V, G, A, S, I, P, Y, or N. In some aspects, U₃ is A, I, Y, G, R, S, V, D, P, or K. In some aspects, U₄ is I, Y, P, V, D, R, K, S, P, or W. In some aspects, U₅ is V, A, G, Y, R, I, W, P, C or S. In some aspects, U₆ is Y, R, V, W, I, V, S, N, P, or D. In some aspects, U₇ is W, S, Y, V, R, I, A, or P. In some aspects, U₈ is S, Y, K, A, P, W, R, N, or D. In some aspects, and U₉ is G, S, A, P, W, R, Y, I, V, or K.

In some aspects, the present disclosure provides a composition comprising a polypeptide comprising the sequence of any one of SEQ ID NOS: 59, 61-66, 68-76, and 78-In some aspects, the present disclosure provides a composition comprising a polypeptide comprising a sequence that is at least 90%, at least 91%, at least 92%, at least 93%, at least 94%, at least 95%, at least 96%, at least 97%, at least 98%, or at least 99% identical to any one of SEQ ID NOS: 59, 61-66, 68-76, and 78-80.

In some aspects, the present disclosure provides a composition comprising a polypeptide comprising a sequence ATGVRAVPGNENSLEIEELARFAVDEHNKKENALLEF VRB₉VKAKEQZ₁Z₂Z₃Z₄Z₅Z₆Z₇Z₈Z₉TMYYLTLEAKDGGKKKLYEAKVWVKU₁U₂U₃U₄U₅U₆U₇U₈U₉NFKELQEFKB₁₀VGDA, wherein each of Z₁-Z₉, each of B₉-B₁₀, and each of U₁-U₉ are independently an amino acid (SEQ ID NO: 105), provided that at least 1, 2, 3, 4, or 5 amino acids of Z₁-Z₉ and U₁-U₉ do not correspond to the amino acid of a wild-type affimer sequence. In some aspects, of U₁-U₉ is independently I, G, A, S, V, W, R, Y, D, K, P, V, or C. In some aspects, each of U₁-U₉ is independently Y, G, S, C, W, R, V, I, D, P, or A. In some aspects, U₁ is Y or W. In some aspects, U₂ is G or V. In some aspects, U₃ is G or I. In some aspects, U₄ is S or R. In some aspects, U₅ is C or G. In some aspects, U₆ is W or D. In some aspects, U₇ is R or S; U₈ is G or P; or U₉ is G or A. In some aspects, each of Z₁-Z₉ is independently Y, I, S, V, D, K, W, A, R, or N. In some aspects, Z₁ is Y or R. In some aspects, Z₂ is I or A. In some aspects, Z₃ is S or R. In some aspects, Z₄ is V or Y. In some aspects, Z₅ is D or Y. In some aspects, Z₆ is K or Y. In some aspects, Z₇ is W or V. In some aspects, Z₈ is V or N. In some aspects, Z₉ is A or R. In some aspects, B₉ is V or M. In some aspects, B₁₀ is P or L.

In some aspects, the present disclosure provides a composition comprising a polypeptide comprising the sequence of any one of SEQ ID NOS: 82-83 and 106. In some aspects, the present disclosure provides a composition comprising a polypeptide comprising a sequence that is at least 90%, at least 91%, at least 92%, at least 93%, at least 94%, at least 95%, at least 96%, at least 97%, at least 98%, or at least 99% identical to any one of SEQ ID NOS: 82-83 and 106.

In some aspects, the present disclosure provides a composition comprising a polypeptide comprising a sequence ATGVRAB₁B₂B₆NENB₃LEIEELARFAVDEHNKKENALL EFVB₇VB₈KAKEQVVAB₄TMYYLTLEAKDB₁₁GKKKLYEAKVWVKU₁U₂U₃U₄U₅U₆U₇U₈U₉NFB₅ELQEFKPVGDA, wherein each of B₁-B₈ and U₁-U₉ are independently an amino acid, provided that at least 1, 2, 3, 4, or 5 amino acids of B₁-B₄ and U₁-U₉ do not correspond to the amino acid of a wild-type affimer sequence. In some aspects, B₁ is M or V. In some aspects, B₂ is S or P. In some aspects, B₃ is G or S. In some aspects, B₄ is C or G. In some aspects, B₅ is R or K. In some aspects, B₆ is G or S. In some aspects, B₇ is R or C. In some aspects, B₈ is V or M. In some aspects, and B₁₁ is G or D.

In some aspects, each of U₁-U₉ is independently I, G, A, S, V, W, R, Y, D, K, P, V, or C. In some aspects, each of U₁-U₉ is independently R, I, V, A, W, S, P, Y, K, or G. In some aspects, U₁ is R, S, or V. In some aspects, U₂ is I, R, P, or W. In some aspects, U₃ is V or W. In some aspects, U₄ is R, Y, or V. In some aspects, U₅ is V or W. In some aspects, U₆ is A, I, P, or S. In some aspects, U₇ is V, R, K, or D. In some aspects, U₈ is W, G, or P. In some aspects, or U₉ is V, G, or A.

In some aspects, the present disclosure provides a composition comprising a polypeptide comprising the sequence of any one of SEQ ID NOS: 85-88, 60, 67, and 77. In some aspects, the present disclosure provides a composition comprising a polypeptide comprising sequence that is at least 90%, at least 91%, at least 92%, at least 93%, at least 94%, at least 95%, at least 96%, at least 97%, at least 98%, or at least 99% identical to any one of SEQ ID NOS: 85-88, 60, 67, and 77.

In some aspects, the present disclosure provides a composition comprising a polypeptide a sequence ATGVRAB₁B₂B₆NENB₃LEIEELARFAVDEHNKKENALLEFVB₇B₉B₈KAKEQZ₁Z₂Z₃Z₄Z₅Z₆Z₇Z₈Z₉TMYYLTLEAKDGGKKKLYEAKVWVKU₁U₂U₃U₄U₅U₆U₇U₈U₉NFB₅ELQEFKB₁₀VGDA, wherein each of B₁, B₂, B₃, B₃-B₁₀, Z₁-Z₉ and U₁-U₉ are independently an amino acid (SEQ ID NO: 107), provided that at least 1, 2, 3, 4, or 5 amino acids of B₁-B₃, B₅-B₁₀, Z₁-Z₉, and U₁-U₉ do not correspond to the amino acid of a wild-type affimer sequence. In some aspects, B₁ is M or V. In some aspects, B₂ is S or P. In some aspects, B₃ is G or S. In some aspects, B₅ is K or R. In some aspects, B₆ is G or S. In some aspects, B₇ is R or C. In some aspects, B₈ is V or M. In some aspects, B₉ is V or M. In some aspects, B₁₀ is P or L. In some aspects, Z₁ is Y, S, or R. In some aspects, Z₂ is I, A, or R. In some aspects, Z₃ is Y, S, or R. In some aspects, Z₄ is P, V, or Y. In some aspects, Z₅ is K, D, or Y. In some aspects, Z₆ is G, K, or Y. In some aspects, Z₇ is N, W, or V. In some aspects, Z₈ is K, V, or N. In some aspects, Z₉ is V, A, or R. In some aspects, U₁ is I, G, A, S, V, W, or A. In some aspects, U₂ is R, V, G, A, S, I, P, Y, or N. In some aspects, U₃ is A, I, Y, G, R, S, V, D, P, or K. In some aspects, U₄ is I, Y, P, V, D, R, K, S, P, or W. In some aspects, U₅ is V, A, G, Y, R, I, W, P, C, or S. In some aspects, U₆ is Y, R, V, W, I, V, S, N, P, or D. In some aspects, U₇ is W, S, Y, V, R, I, A, or P. In some aspects, U₈ is S, Y, K, A, P, W, R, N, or D. In some aspects, U₉ is G, S, A, P, W, R, Y, I, V, or K.

In some aspects, the present disclosure provides a composition comprising a polypeptide comprising a sequence VDNKFNKEJ₁J₂J₃AJ₄J₅EIJ₆J₇LPNLNJ₈J₉QJ₁₀J₁₁AFLJ₁₂S J₁₇J₁₃DDPJ₁₄J₁₅J₁₆ANLLAEAKKLNDAQAPK, wherein each of J₁-J₁₆ is independently an amino acid or absent (SEQ ID NO: 108), provided that at least 1, 2, 3, 4, or 5 amino acids of J₁-J₁₆ do not correspond to the amino acid of a wild-type affibody sequence.

In some aspects, the present disclosure provides a composition comprising a polypeptide wherein each of J₁-J₁₇ are independently Q, W, L, Y, H, A, M, R, E, K, V, S, Q, R, or P. In some aspects, J₁ is Q, W, Y, E, L, M, K, or S. In some aspects, J₂ is W, Y, S, L, A, M, Q, V, or R. In some aspects, J₃ is L, A, V, M, Y, W, or H. In some aspects, J₄ is W, E, M, H, V, A, or Y. In some aspects, J₅ is Y, L, M, V, S, or A. In some aspects, J₆ is H, W, Y, E, L, Q, A, Y, M, R, or S. In some aspects, J₇ is Y, W, L, R, S, E, or A. In some aspects, J₈ is A, M, S, L, or H. In some aspects, J₉ is M, K, Y, Q, W, or L. In some aspects, J₁₀ is H, W, L, M, E, S, A, or absent. In some aspects, J₁₁ is R, Y, V, A, L, or E. In some aspects, J₁₂ is L, Y, V, W, E, M, or Q. In some aspects, J₁₃ is Y, E, V, R, W, Q, or H. In some aspects, J₁₄ is R or S. In some aspects, J₁₅ is R or Q. In some aspects, Jib is H, S, P, or R. In some aspects, J₁₁ is L or P.

In some aspects, the present disclosure provides a composition comprising a polypeptide comprising the sequence of any one of SEQ ID NOS: 91-103. In some aspects, the present disclosure provides a composition comprising a polypeptide comprising a sequence that is at least 90%, at least 91%, at least 92%, at least 93%, at least 94%, at least 95%, at least 96%, at least 97%, at least 98%, or at least 99% identical to any one of SEQ ID NOS: 91-103.

In some aspects, the present disclosure provides a composition comprising a polypeptide comprising an amino acid sequence with at least 90%, at least 91%, at least 92%, at least 93%, at least 94%, at least 95%, at least 96%, at least 97%, at least 98%, or at least 99% identity to any one of SEQ ID NOS: 55-57, 59-80, 82, 83, 85-88, 91-103, and 106.

In some embodiments of the foregoing or related aspects, the disclosure provides a composition comprising a multimeric polypeptide comprising at least one polypeptide comprising a sequence disclosed herein or comprising a sequence of SEQ ID NOs: 54-80, 82, 83, 85-88, 91-103, 105-107, and a polypeptide linker.

In some embodiments of the foregoing or related aspects, the disclosure provides a composition comprising a multimeric polypeptide comprising from about 1 to about 10 polypeptides of any one of a polypeptide comprising a sequence disclosed herein or comprising a sequence of SEQ ID NOs: 54-80, 82, 83, 85-88, 91-103, 105-107, and from about 1 to about polypeptide linkers.

In some embodiments of the foregoing or related aspects, the disclosure provides a composition comprising a multimeric polypeptide comprising from N- to C-terminus: a first polypeptide linker, a first polypeptide comprising a sequence disclosed herein or comprising a sequence of SEQ ID NOs: 54-80, 82, 83, 85-88, 91-103, 105-107.

The multimeric polypeptide comprising a second polypeptide linker that is C-terminal to the first polypeptide of any one of a polypeptide comprising a sequence disclosed herein or comprising a sequence of SEQ ID NOs: 54-80, 82, 83, 85-88, 91-103, 105-107 and a second polypeptide comprising a sequence disclosed herein or comprising a sequence of SEQ ID NOs: 54-80, 82, 83, 85-88, 91-103, 105-107, wherein the second polypeptide comprising a sequence disclosed herein or comprising a sequence of SEQ ID NOs: 54-80, 82, 83, 85-88, 91-103, 105-107 is C-terminal to the second polypeptide linker comprising a sequence disclosed herein or comprising a sequence of SEQ ID NOs: 54-80, 82, 83, 85-88, 91-103, 105-107.

In some aspects, the present disclosure provides a composition comprising a multimeric polypeptide having an amino acid sequence with at least 90%, at least 91%, at least 92%, at least 93%, at least 94%, at least 95%, at least 96%, at least 97%, at least 98%, at least 99%, or 100% identity to any one of SEQ ID NOS: 169-193.

In some aspects, the present disclosure provides a composition comprising a nucleic acid encoding a polypeptide of a polypeptide comprising a sequence disclosed herein or comprising a sequence of SEQ ID NOs: 54-80, 82, 83, 85-88, 91-103, 105-107. In some aspects, the present disclosure provides a composition comprising a nucleic acid encoding a multimeric polypeptide comprising at least one of a polypeptide comprising a sequence disclosed herein or comprising a sequence of SEQ ID NOs: 54-80, 82, 83, 85-88, 91-103, 105-107, 169-193.

In some aspects, the present disclosure provides a method of binding an AAV capsid. In some aspects, the present disclosure provides a method of binding an AAV capsid, comprising contacting an AAV capsid with a polypeptide comprising a sequence disclosed herein or comprising a sequence of SEQ ID NOs: 54-80, 82, 83, 85-88, 91-103, 105-107 or a multimeric polypeptide comprising at least one of a polypeptide comprising a sequence disclosed herein or comprising a sequence of SEQ ID NOs: 54-80, 82, 83, 85-88, 91-103, 105-107, 169-193. In some aspects, the AAV is AAV9. In some aspects, the AAV capsid is full.

In some aspects, the present disclosure provides a method of separating full AAV capsids from empty AAV capsids. In some aspects, the present disclosure provides a method of separating full AAV capsids from empty AAV capsids, the method comprising: (a) contacting a polypeptide with a solution comprising the mixture of full AAV capsids and empty AAV capsids, wherein the polypeptide shows at least a two-fold increase in binding preference for full AAV capsids versus empty AAV capsids; (b) allowing the polypeptide to bind the full AAV capsids; (c) separating empty AAV capsids from the full AAV capsids bound to the polypeptide. In some aspects, the polypeptide comprises an avimer, affimer, or affibody. In some aspects, the polypeptide is a polypeptide comprising a sequence disclosed herein or comprising a sequence of SEQ ID NOs: 54-80, 82, 83, 85-88, 91-103, 105-107 or a multimeric polypeptide comprising at least one of a polypeptide comprising a sequence disclosed herein or comprising a sequence of SEQ ID NOs: 54-80, 82, 83, 85-88, 91-103, 105-107, 169-193.

In some aspects, the present disclosure provides a composition comprising a nucleic acid with at least 90%, at least 91%, at least 92%, at least 93%, at least 94%, at least 95%, at least 96%, at least 97%, at least 98%, at least 99%, or 100% identity to any one of SEQ ID NOs: 194-195.

These and other embodiments will be further described below in the Detailed Description, Examples, and Claims.

### BRIEF DESCRIPTION OF THE DRAWINGS

FIG. 1 shows the binding of the polypeptides of Example 1 to AAV9 capsids compared to human serum albumin (HSA). In comparison to a control polypeptide (i.e. the human polycystic kidney domain 2, which is known to bind to AAV9 capsids), 34 polypeptides had higher specificity for AAV9 capsids.

FIG. 2 shows the luminescence intensity of the polypeptides of Example 1 bound to AAV9 capsids. The intensity is normalized to the intensity of the control sequence (i.e. the human polycystic kidney domain 2, which is known to bind to AAV9 capsids). Many polypeptides exhibited more than 10× the binding signal of the control sequence.

FIGS. 3A-3D shows the binding of seven of the polypeptides of Example 1 to AAV2 (FIG. 3A), AAV5 (FIG. 3B), AAV8 (FIG. 3C), and AAV9 (FIG. 3D) capsids. The control sequence (i.e. the human polycystic kidney domain 2, which is known to bind to AAV9 capsids) is labeled “C” in each graph.

FIG. 4 shows that the five of the polypeptides of Example 1 are sensitive to acidic pH (pH 3, pH 4, pH 5). The graph shows that each clone binds with a lower intensity at acidic pH than at neutral pH (pH 7.2).

FIG. 5 shows the full capsid bias index (FCBI) of select polypeptides of Example 1. The selected polypeptides selectively bind to full capsids over empty capsids. The control sequence (i.e. the human polycystic kidney domain 2, which is known to bind to AAV9 capsids) is labeled “C” in each graph. The control ligand is known to bind to AAV9, but is not known to discriminate between empty and full AAV particles. The equation for calculating FCBI is: FCBI=((AAV Full Capsids RLU of Sample)/(AAV Full Capsids RLU of Control))/((AAV Empty Capsids RLU of Sample)/(AAV Empty Capsids RLU of Control)).

FIG. 6 shows the specificity ratio (SR) for each of the polypeptides displayed in FIG. 5. Each of polypeptides 1-7 preferentially bind to full AAV capsids compared to human serum albumin. The AAV SR is calculated using the following equation: AAV SR=(AAV full capsids RLU)/(human serum albumin RLU).

FIG. 7A shows the RLU for binding of polypeptides 1-7 of FIG. 5 for AAV9 full capsids, empty capsids, or human serum albumin. The polypeptide concentration was 900 nM.

FIG. 7B shows the RLU for binding of polypeptides 1-7 of FIG. 5 for AAV9 full capsids, empty capsids, or human serum albumin. The polypeptide concentration was 300 nM.

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “about” or “approximately” when preceding a numerical value indicates the value plus or minus a range of 10%. For example, “about 100” encompasses 90 and 110.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features described herein can be used in any combination.

Moreover, the present disclosure also contemplates that in some embodiments, any feature or combination of features set forth herein can be excluded or omitted. To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc., as if each such subcombination is expressly set forth herein. Moreover, such language also indicates that one or more of the specified amino acids can be disclaimed. For example, in particular embodiments the amino acid is not A, G or I; is not A; is not G or V; etc., as if each such possible disclaimer is expressly set forth herein.

An “adeno-associated virus” (AAV) is a small, replication-deficient parvovirus. As used herein, AAV may refer to a wildtype or mutant AAV of any one of the following serotypes: AAV1, AAV2, AAV3 (including types 3A and 3B), AAV4, AAV5, AAV6, AAV7, AAV8, AAV9, AAV10, AAV11, AAV12, AAV13, AAVrh32.33, AAVrh8, AAVrh10, AAVrh74, AAVhu.68, avian AAV, bovine AAV, canine AAV, equine AAV, ovine AAV, snake AAV, bearded dragon AAV, AAV2i8, AAV2g9, AAV-LK03, AAV7m8, AAV Anc80, AAV PHP.B, and any other AAV now known or later discovered. In some embodiments, an AAV may have a single-stranded genome, or a double-stranded genome (e.g., a self-complementary AAV).

An “AAV particle” typically comprises a capsid, and a nucleic acid (e.g., a nucleic acid comprising a transgene) encapsidated by the protein capsid. The “capsid” is a near-spherical protein shell that comprises individual “capsid protein subunits” or “capsid proteins” (e.g., about 60 capsid protein subunits) associated and arranged with T=1 icosahedral symmetry. Accordingly, the capsids of the AAV vectors described herein comprise a plurality of capsid proteins. When an AAV particle is described as comprising a capsid protein, it will be understood that the AAV particle comprises a capsid, wherein the capsid comprises one or more AAV capsid proteins. When an AAV particle is described as binding to a binding domain, it will be understood that the binding domain may bind to one or more capsid proteins within the capsid. The term “empty AAV particle” or “empty capsid” refers to an AAV particle or capsid that does not comprise any vector genome or nucleic acid comprising an expression cassette or transgene. The term “full AAV particle” or “full capsid” refers to an AAV particle or capsid that comprises a vector genome or nucleic acid comprising an expression cassette or transgene. The term “partial AAV particle” or “partial capsid” refers to an AAV particle or capsid that comprises part of a vector genome or nucleic acid comprising an expression cassette or transgene.

As used herein, the term “AAV sample” used interchangeably herein with “AAV composition” refers to a composition that contains AAV particles. In some embodiments, the “AAV sample” refers to a composition containing AAV of a particular serotype. For example, an “AAV8 sample” refers to a composition comprising AAV8 particles.

As used herein, the term “contaminant” and “impurity” are used interchangeably. A contaminant may refer to any substance that is not desired in a purified composition. In some embodiments, the contaminant is any substance other than the biologic desired to be purified. Non-limiting examples of contaminants include, but are not limited to, a solvent, a protein, a peptide, a carbohydrate, a nucleic acid, a virus, a cell (e.g., a bacterial, yeast, or mammalian cell), a carbohydrate, a lipid, or a lipopolysaccharide. In some embodiments, the contaminant is an endotoxin or a mycotoxin. In some embodiments, the contaminant is empty or partial AAV particles or capsids.

As used herein, the term “fragment” as it refers to a protein or polypeptide includes a truncated form of the protein or polypeptide. For example, a fragment of AAVR may include at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% of the amino acids of full-length AAVR.

As used herein, the terms “peptide,” “polypeptide,” and “protein” are used interchangeably, and refer to a compound comprised of amino acid residues covalently linked by peptide bonds. A protein must contain at least two amino acids, and no limitation is placed on the maximum number of amino acids that can comprise a protein's sequence. The term “peptide” may refer to a short chain of amino acids including, for example, natural peptides, recombinant peptides, synthetic peptides, or a combination thereof. Proteins and peptides may include, for example, biologically active fragments, substantially homologous polypeptides, oligopeptides, homodimers, heterodimers, variants of polypeptides, modified polypeptides, derivatives, analogs, and fusion proteins, among others.

A “polynucleotide” is a sequence of nucleotide bases, and may be RNA, DNA or DNA-RNA hybrid sequences (including both naturally occurring and non-naturally occurring nucleotides). In some embodiments, a polynucleotide is either a single or double stranded DNA sequence.

As used herein, by “isolate” or “purify” (or grammatical equivalents) a viral particle, it is meant that the viral particle is at least partially separated from at least some of the other components in a starting material comprising the viral particle (e.g., a cell lysate). In representative embodiments an “isolated” or “purified” viral particle is enriched by at least about 10-fold, about 100-fold, about 1000-fold, about 10,000-fold or more as compared with the starting material.

As used herein, the term “amino acid” encompasses any naturally occurring amino acid, modified forms thereof, and synthetic amino acids.

The term “percent identity” in the context of two or more nucleic acid or polypeptide sequences, refers to two or more sequences or subsequences that have a specified percentage of nucleotides or amino acid residues that are the same, when compared. Unless otherwise indicated, percent identity is determined using the National Center for Biotechnology Information (NCBI)'s Basic Local Alignment Search Tool (BLAST®), available at blast.ncbi.nlm.nih.gov/Blast.cgi, version BLAST+2.13.0. In some embodiments, the percent identity is calculated over the entire length of the compared sequences. In some embodiments, the sequence identity is calculated over a fragment of each compared sequence of about 10 amino acids, about 15 amino acids, about 20 amino acids, about 25 amino acids, about 30 amino acids, about 35 amino acids, about 40 amino acids, about 45 amino acids, about 50 amino acids, about 55 amino acids, about 60 amino acids, about 65 amino acids, about 70 amino acids, about 75 amino acids, about 80 amino acids, about 85 amino acids, about 90 amino acids, about 95 amino acids, about 100 amino acids, about 105 amino acids, about 110 amino acids, about 115 amino acids, about 120 amino acids, about 125 amino acids, about 130 amino acids, about 135 amino acids, about 140 amino acids, about 145 amino acids, about 150 amino acids, about 155 amino acids, about 160 amino acids, about 165 amino acids, about 170 amino acids, about 175 amino acids, about 180 amino acids, about 185 amino acids, about 190 amino acids, about 195 amino acids, or about 200 amino acids.

### Compositions Comprising AAV-Binding Polypeptides

The disclosure provides novel adeno-associated virus (AAV)-binding polypeptides. In some embodiments, the novel AAV-binding polypeptides bind preferentially to full AAV particles or capsids. In some embodiments, the novel AAV-binding polypeptides bind to viral particles containing a capsid comprising one or more of viral protein 1 (VP1), viral protein 2 (VP2), and viral protein 3 (VP3).

### Polypeptides Based on an Aylmer Scaffold

In some embodiments, the compositions provided herein comprise polypeptides comprising a sequence CX₁X₂X₃X₄FX₅CX₆X₇X₈X₉X₁₀CX₁₁X₁₂X₁₃TWVCDGX₁₄X₁₅DCX₁₆DX₁₇X₁₈DEX₁₉X₂₀CTPTP (SEQ ID NO: 54), wherein each of X₁-X₂₀ are independently an amino acid. In embodiments, at least 1, 2, 3, 4, or 5 amino acids of X₁-X₂₀ do not correspond to the amino acid of a wild-type avimer sequence. In embodiments, at least 1, 2, 3, 4, 5, 6, 7, 8, 9, 10, 11, 12, 13, 14, 15, 16, 17, 18, or 19 amino acids (or any range therein) of X₁-X₂₀ do not correspond to the amino acid of a wild-type avimer sequence. In embodiments, 1, 2, 3, 4, 5, 6, 7, 8, 9, 10, 11, 12, 13, 14, 15, 16, 17, 18, 19, or 20 amino acids (or any range therein) of X₁-X₂₀ do not correspond to the amino acid of a wild-type avimer sequence. In embodiments, provided herein are polypeptides with between 1-5, between 1-10, or between 1-20 amino acids, i.e., about 1, 2, 3, 4, 5, 6, 7, 8, 9, 10, 11, 12, 13, 14, 15, 16, 17, 18, 19, or 20 amino acids, which are mutated, deleted, or inserted, as compared to the polypeptide of SEQ ID NO: 54. Table 1A contains wild-type avimer sequences. In embodiments, the mutations, deletions, or insertions may occur at amino acid positions other than X₁-X₂₀ of the polypeptide of SEQ ID NO: 54.

TABLE 1A

| | SEQ |
|---|---|
| | ID |
| Polypeptide | NO: |
| TCSQDEFRCHDGK CISRQFVCDSDRDCLDGSDEASCP | 6 |
| TCKSGDFSCGGRVNRCIPQFWRCDGQVDCDNGSDEQG | 7 |

In embodiments, each of X₁-X₁₉ are amino acids independently selected from D, I, P, V, W, S, A, K, R, N, or G. In embodiments, each of X₁-X₄ are independently D, I, P, W, V, K, I, A, or N. In embodiments, X₁ is D, W, or A; X₂ is I or V; X₃ is P, K, or D; and X₄ is P, I, or N. In embodiments, X₅ is V, R, or P. In embodiments, each of X₆-X₁₀ are independently selected from W, P, S, N, I, V, G, W, Y, or D. In embodiments, X₆ is W or N; X₇ is W or I; X₈ is P, V, or Y; X₉ is S, G, or Y; and X₁₀ is W, V, or D. In embodiments, each of X₁₁, X₁₂, and X₁₃ are independently V, I, W, A, or G. In embodiments, X₁₁ is V or I; X₁₂ is W, V, or G; and X₁₃ is A or N. In embodiments, each of X₁₄ and X₁₅ are independently A, P, R, V, or I. In embodiments, X₁₆ is I or S. In embodiments, X₁₇ and X₁₈ are independently N, Y, P, I, or A. In embodiments, X₁₇ is N, P, or I; and X₁₈ is Y, P, or A. In embodiments, each of X₁₉ and X₂₀ are independently W, I, G, Y or D. In embodiments, X₁₉ is W, G, or Y; and zX₂₀ is I, W, or D.

In embodiments, the polypeptide comprises a sequence selected from Table 1B or a polypeptide with at least 80%, at least 85%, at least 90%, at least 91%, at least 92%, at least 93%, at least 94%, at least 95%, at least 96%, at least 97%, at least 98%, at least 99%, or 100% identity to the sequence of Table 1B.

TABLE 1B

| | SEQ |
|---|---|
| | ID |
| Polypeptide | NO: |
| CDIPPFVCWWPSWCVWATWVCDGAPDCIDNYDEWICTPTP | 55 |
| CWVKIFRCNIVGVCIVNTWVCDGRVDCSDPPDEGWCTPTP | 56 |
| CAIDNFPCNWYIDCIGATWVCDGIIDCIDIADEYDCTPTP | 57 |

### Polypeptides Based on an Affimer: Loop 1 Scaffold

In some embodiments, the compositions provided herein comprise polypeptides comprising the sequence ATGVRAVPGNENSLEIEELARFAVDEHNKKENALLEFVRVVKAKEQVVAG TMYYLTLEAKDGGKKKLYEAKVWVKU₁U₂U₃U₄U₃U₆U₇U₈U₉NFKELQEFKPVGDA (SEQ ID NO: 58). In embodiments, at least 1, 2, 3, 4, or 5 amino acids of U₁-U₉ do not correspond to the amino acid of a wild-type affimer sequence. In embodiments, at least 1, 2, 3, 4, 6, 7, or 9 amino acids of U₁-U₉ do not correspond to the amino acid of a wild-type affimer sequence. In embodiments, 1, 2, 3, 4, 6, 7, 9, or 10 amino acids of U₁-U₉ do not correspond to the amino acid of a wild-type affimer sequence. In embodiments, provided herein are polypeptides with between 1-5 or between 1-10 or between 1-20 amino acids, i.e., about 1, 2, 3, 4, 5, 6, 7, 8, 9, 10, 11, 12, 13, 14, 15, 16, 17, 18, 19, or 20 amino acids, which are mutated, deleted, or inserted, as compared to the polypeptide of SEQ ID NO: 58. The wild-type affimer sequence is provided in Table 2A. In embodiments, the mutations, deletions, or insertions may occur at amino acid positions other than U₁-U₉ of the polypeptide of SEQ ID NO: 58.

TABLE 2A

| | SEQ |
|---|---|
| | ID |
| Polypeptide | NO: |
| ATGVRAVPGNENSLEIEELARFAVD | 8 |
| EHNKKENALLEFVRVVKAKEQVVAG | |
| TMYYLTLEAKDGGKKKLYEAKVWVK | |
| PWE NFKELQEFKPVGDA | |

In embodiments, each of U₁-U₉ are independently I, G, A, S, V, W, R, Y, D, K, P, V, K or C. In embodiments, U₁ is I, G, A, S, V, W, or A. In embodiments, U₂ is R, V, G, A, S, I, P, Y, or N. In embodiments, U₃ is A, I, Y, G, R, S, V, D, P, or K. In embodiments, U₄ is I, Y, P, V, D, R, K, S, P, or W. In embodiments, U₅ is V, A, G, Y, R, I, W, P, C or S. In embodiments, U₆ is Y, R, V, W, I, V, S, N, P, or D. In embodiments, U₇ is W, S, Y, V, R, I, A, or P. In embodiments, U₈ is S, Y, K, A, P, W, R, N, or D. In embodiments, U₉ is G, S, A, P, W, R, Y, I, V, or K.

In embodiments, the polypeptide comprises an amino acid sequence selected from Table 2B or a polypeptide with at least 80%, at least 85%, at least 90%, at least 91%, at least 92%, at least 93%, at least 94%, at least 95%, at least 96%, at least 97%, at least 98%, at least 99%, or 100% identity to an amino acid sequence of Table 2B.

TABLE 2B

| Polypeptide | SEQ ID NO: |
|---|---|
| ATGVRAVPGNENSLEIEELARFAVDEHNKKENALLEFVRVVKAKEQ | 59 |
| VVAGTMYYLTLEAKDGGKKKLYEAKVWVKIRAIVYWSGNFKELQ | |
| EFKPVGDA | |
| ATGVRAVPGNENSLEIEELARFAVDEHNKKENALLEFVRVVKAKEQ | 61 |
| VVAGTMYYLTLEAKDGGKKKLYEAKVWVKAGYIGRSKANFKEL | |
| QEFKPVGDA | |
| ATGVRAVPGNENSLEIEELARFAVDEHNKKENALLEFVRVVKAKEQ | 62 |
| VVAGTMYYLTLEAKDGGKKKLYEAKVWVKAAGYYVYAPNFKEL | |
| QEFKPVGDA | |
| ATGVRAVPGNENSLEIEELARFAVDEHNKKENALLEFVRVVKAKEQ | 63 |
| VVAGTMYYLTLEAKDGGKKKLYEAKVWVKISRPGWWPWNFKEL | |
| QEFKPVGDA | |
| ATGVRAVPGNENSLEIEELARFAVDEHNKKENALLEFVRVVKAKEQ | 64 |
| VVAGTMYYLTLEAKDGGKKKLYEAKVWVKSRSVRIWYSNFKELQ | |
| EFKPVGDA | |
| ATGVRAVPGNENSLEIEELARFAVDEHNKKENALLEFVRVVKAKEQ | 65 |
| VVAGTMYYLTLEAKDGGKKKLYEAKVWVKVRRDIVVPRNFKELQ | |
| EFKPVGDA | |
| ATGVRAVPGNENSLEIEELARFAVDEHNKKENALLEFVRVVKAKEQ | 66 |
| VVAGTMYYLTLEAKDGGKKKLYEAKVWVKVRDIVSIRGNFKELQ | |
| EFKPVGDA | |
| ATGVRAVPGNENSLEIEELARFAVDEHNKKENALLEFVRVVKAKEQ | 68 |
| VVAGTMYYLTLEAKDGGKKKLYEAKVWVKSASYYVYSYNFKELQ | |
| EFKPVGDA | |
| ATGVRAVPGNENSLEIEELARFAVDEHNKKENALLEFVRVVKAKEQ | 69 |
| VVAGTMYYLTLEAKDGGKKKLYEAKVWVKWRAVRWWWGNFK | |
| ELQEFKPVGDA | |
| ATGVRAVPGNENSLEIEELARFAVDEHNKKENALLEFVRVVKAKEQ | 70 |
| VVAGTMYYLTLEAKDGGKKKLYEAKVWVKVVGKWWVNINFKE | |
| LQEFKPVGDA | |
| ATGVRAVPGNENSLEIEELARFAVDEHNKKENALLEFVRVVKAKEQ | 71 |
| VVAGTMYYLTLEAKDGGKKKLYEAKVWVKARYVVYYRWNFKEL | |
| QEFKPVGDA | |
| ATGVRAVPGNENSLEIEELARFAVDEHNKKENALLEFVRVVKAKEQ | 72 |
| VVAGTMYYLTLEAKDGGKKKLYEAKVWVKGRYSVYWYVNFKEL | |
| QEFKPVGDA | |
| ATGVRAVPGNENSLEIEELARFAVDEHNKKENALLEFVRVVKAKEQ | 73 |
| VVAGTMYYLTLEAKDGGKKKLYEAKVWVKVRYPPNIWKNFKEL | |
| QEFKPVGDA | |
| ATGVRAVPGNENSLEIEELARFAVDEHNKKENALLEFVRVVKAKEQ | 74 |
| VVAGTMYYLTLEAKDGGKKKLYEAKVWVKVAAYPRASINFKELQ | |
| EFKPVGDA | |
| ATGVRAVPGNENSLEIEELARFAVDEHNKKENALLEFVRVVKAKEQ | 75 |
| VVAGTMYYLTLEAKDGGKKKLYEAKVWVKIGIPIRIWSNFKELQE | |
| FKPVGDA | |
| ATGVRAVPGNENSLEIEELARFAVDEHNKKENALLEFVRVVKAKEQ | 76 |
| VVAGTMYYLTLEAKDGGKKKLYEAKVWVKVNIYVISPRNFKELQ | |
| EFKPVGDA | |
| ATGVRAVPGNENSLEIEELARFAVDEHNKKENALLEFVRVVKAKEQ | 78 |
| VVAGTMYYLTLEAKDGGKKKLYEAKVWVKSVAIRNWYWNFKEL | |
| QEFKPVGDA | |
| ATGVRAVPGNENSLEIEELARFAVDEHNKKENALLEFVRVVKAKEQ | 79 |
| VVAGTMYYLTLEAKDGGKKKLYEAKVWVKVRRYSPPNRNFKEL | |
| QEFKPVGDA | |
| AATGVRAVPGNENSLEIEELARFAVDEHNKKENALLEFVRVVKAKE | 80 |
| QVVAGTMYYLTLEAKDGGKKKLYEAKVWVKVRYWVDRRKNFK | |
| ELQEFKPVGDA | |

Polypeptides based on an Affimer: Loop 2 Scaffold

In embodiments, provided herein are polypeptides comprising a sequence ATGVRAVPGNENSLEIEELARFAVDEHNKKENALLEFVRVVKAKEQZ₁Z₂Z₃Z₄Z₅Z₆Z₇Z₈Z₉TMYYLTLEAKDGGKKKLYEAKVWVKU₁U₂U₃U₄U₅U₆U₇U₈U₉NEKELQEFKPVG DA, wherein each of Z₁-Z₉ and each of U₁-U₉ are independently an amino acid. (SEQ ID NO: 81). In embodiments, at least 1, 2, 3, 4, or 5 amino acids of U₁-U₉ and Z₁-Z₉ do not correspond to the amino acid of a wild-type affimer sequence. In embodiments, at least 1, 2, 3, 4, 5, 6, 7, 8, 9, 10, 11, 12, 13, 14, 15, 16, or 17 amino acids (or any ranges therein) of U₁-U₉ and Z₁-Z₉ do not correspond to the amino acid of a wild-type affimer sequence. In embodiments, provided herein are polypeptides with between 1-5 or between 1-10 or between 1-20 amino acids, i.e., about 1, 2, 3, 4, 5, 6, 7, 8, 9, 10, 11, 12, 13, 14, 15, 16, 17, 18, 19, or 20 amino acids, which are mutated, deleted, or inserted, as compared to the polypeptide of SEQ ID NO: 81. In embodiments, the mutations, deletions, or insertions may occur at amino acid positions other than U₁-U₉ and Z₁-Z₉ of the polypeptide of SEQ ID NO: 81. The wild-type affimer sequence is provided in Table 2A.

In embodiments, provided herein are polypeptides comprising a sequence ATGVRAVPGNENSLEIEELARFAVDEHNKKENALLEFVRB₉VKAKEQZ₁Z₂Z₃Z₄Z₅Z₆Z 7Z₈Z₉TMYYLTLEAKDGGKKKLYEAKVWVKU₁U₂U₃U₄U₅U₆U₇U₈U₉NEKELQEFKB₁₀V GDA, wherein each of Z₁-Z₉, each of B₉ and B₁₀, and each of U₁-U₉ are independently an amino acid (SEQ ID NO: 105). In embodiments, at least 1, 2, 3, 4, or 5 amino acids of U₁-U₉, B₉, B₁₀, and Z₁-Z₉ do not correspond to the amino acid of a wild-type affimer sequence. In embodiments, at least 1, 2, 3, 4, 5, 6, 7, 8, 9, 10, 11, 12, 13, 14, 15, 16, 17, 18, or 19 amino acids of U₁-U₉, B₉, B₁₀, and Z₁-Z₉ do not correspond to the amino acid of a wild-type affimer sequence. In embodiments, 1, 2, 3, 4, 5, 6, 7, 8, 9, 10, 11, 12, 13, 14, 15, 16, 17, 18, 19, or 20 amino acids (or ranges therein) of U₁-U₉, B₉, B₁₀, and Z₁-Z₉ do not correspond to the amino acid of a wild-type affimer sequence. In embodiments, provided herein are polypeptides with between 1-5, between 1-10 or between 1-20 amino acids, e.g., about 1, 2, 3, 4, 5, 6, 7, 8, 9, 10, 11, 12, 13, 14, 15, 16, 17, 18, 19, or 20 amino acids, which are mutated, deleted, or inserted, as compared to the polypeptide of SEQ ID NO: 105. In embodiments, the mutations, deletions, or insertions may occur at amino acid positions other than B₉, B₁₀, U₁-U₉ and Z₁-Z₉ of the polypeptide of SEQ ID NO: 105. The wild-type affimer sequence is provided in Table 2A.

In embodiments, each of U₁-U₉ are independently I, G, A, S, V, W, R, Y, D, K, P, V, K or C. In embodiments, U₁ is I, G, A, S, V, W, or A. In embodiments, U₂ is R, V, G, A, S, I, P, Y, or N. In embodiments, U₃ is A, I, Y, G, R, S, V, D, P, or K. In embodiments, U₄ is I, Y, P, V, D, R, K, S, P, or W. In embodiments, U₅ is V, A, G, Y, R, I, W, P, C or S. In embodiments, U₆ is Y, R, V, W, I, V, S, N, P, or D. In embodiments, U₇ is W, S, Y, V, R, I, A, or P. In embodiments, U₈ is S, Y, K, A, P, W, R, N, or D. In embodiments, U₉ is G, S, A, P, W, R, Y, I, V, or K.

In embodiments, each of U₁-U₉ is independently Y, G, S, C, W, R, V, I, D, P, or A. In embodiments, U₁ is Y or W; U₂ is G or V; U₃ is G or I; U₄ is S or R; U₅ is C or G; U₆ is W or D; U₇ is R or S; U₈ is G or P; or U₉ is G or A. In embodiments, U₁ is V, Y, or W; U₂ is A, G, or V; U₃ is V, G, or I; U₄ is V, S, or R; U₅ is A, C, or G; U₆ is R, W, or D; U₇ is A, R, or S; U₈ is A, G, or P; or U₉ is V, G, or A.

In embodiments, B₉ is V or M. In embodiments, B₁₀ is P or L.

In embodiments, each of Z₁-Z₉ is independently Y, I, S, V, D, K, W, A, R, or N. In embodiments, Z₁ is Y or R. In embodiments, Z₂ is I or A. In embodiments, Z₃ is S or R. In embodiments, Z₄ is V or Y. In embodiments, Z₅ is D or Y. In embodiments, Z₆ is K or Y. In embodiments, Z₇ is W or V. In embodiments, Z₈ is V or N. In embodiments, Z₉ is A or R. In embodiments, Z₁ is Y or R. In embodiments, Z₂ is I or A. In embodiments, Z₃ is S or R. In embodiments, Z₄ is V or Y. In embodiments, Z₅ is D or Y. In embodiments, Z₆ is K or Y. In embodiments, Z₇ is W or V. In embodiments, Z₈ is V or N. In embodiments, Z₉ is A or R. In embodiments, Z₁ is S or R. In embodiments, Z₂ is R or A. In embodiments, Z₃ is Y or R. In embodiments, Z₄ is P or Y. In embodiments, Z₅ is K or Y. In embodiments, Z₆ is G or Y. In embodiments, Z₇ is N or V. In embodiments, Z₈ is K or N. In embodiments, Z₉ is V or R.

In embodiments, the polypeptide comprises a sequence selected from Table 2C or a polypeptide with at least 80%, at least 85%, at least 90%, at least 91%, at least 92%, at least 93%, at least 94%, at least 95%, at least 96%, at least 97%, at least 98%, at least 99%, or 100% identity to the sequence of any one of the polypeptides of Table 2C.

TABLE 2C

| | SEQ |
|---|---|
| | ID |
| Polypeptide | NO: |
| ATGVRAVPGNENSLEIEELARFAVDEHNKK | 82 |
| ENALLEFVRMVKAKEQYISVDKWVATMYYL | |
| TLEAKDGGKKKLYEAKVWVKYGGSCWRGGN | |
| FKELQEFKPVGDA | |
| ATGVRAVPGNENSLEIEELARFAVDEHNKK | 83 |
| ENALLEFVRVVKAKEQRARYYYVNRTMYYL | |
| TLEAKDGGKKKLYEAKVWVKWVIRGDSPAN | |
| FKELQEFKLVGDA | |
| ATGVRAVPGNENSLEIEELARFAVDEHNKK | 106 |
| ENALLEFVRVVKAKEQSRYPKGNKVTMYYL | |
| TLEAKDGGKKKLYEAKVWVKVAVVARAAVN | |
| FKELQEFKPVGDA | |

Polypeptides Based on an Affimer: Loop 1 Scaffold with Additional Mutations

In embodiments, the compositions provided herein comprise polypeptides comprising the sequence: ATGVRAB₁B₂GNENB₃LEIEELARFAVDEHNKKENALLEFVRVVKAKEQVVAB₄ TMYYLTLEAKDGGKKKLYEAKVVVVKU₁U₂U₃U₄U₃U₆U₇U₈U₉NFKELQEFKPVGDA (SEQ ID NO: 84), wherein each of B₁, B₂, B₃, Ba, and U₁-U₉ are independently an amino acid. In embodiments, at least 1, 2, 3, 4, or 5 amino acids of B₁, B₂, B₃, Ba, and U₁-U₉ do not correspond to the amino acid of a wild-type affimer sequence. In embodiments, at least 1, 2, 3, 4, 5, 6, 7, 8, 9, 10, 11, or 12 amino acids (or combinations thereof) of B₁, B₂, B₃, Ba, and U₁-U₉ do not correspond to the amino acid of a wild-type affimer sequence. In embodiments, 1, 2, 3, 4, 5, 6, 7, 8, 9, 10, 11, 12, or 13 amino acids (or combinations thereof) of B₁, B₂, B₃, Ba, and U₁-U₉ do not correspond to the amino acid of a wild-type affimer sequence. In embodiments, provided herein are polypeptides with between 1-5, between 1-10, or between 1-13 amino acids, e.g., about 1, 2, 3, 4, 5, 6, 7, 8, 9, 10, 11, 12, or 13 amino acids, which are mutated, deleted, or inserted, as compared to the polypeptide of SEQ ID NO: 84. In embodiments, the mutations, deletions, or insertions may occur at amino acid positions other than B₁-134 and U₁-U₉ of the polypeptide of SEQ ID NO: 84. The wild-type affimer sequence is provided in Table 2A.

In embodiments, the compositions provided herein comprise polypeptides comprising the sequence: ATGVRAB₁B₂B₆NENB₃LEIEELARFAVDEHNKKENALLEFV B₇VB₈KAKEQVVABaTMYYLTLEAKDBliGKKKLYEAKVVVVKU₁U₂U₃U₄U₃U₆U₇U₈U₉ NFB₅ELQEFKPVGDA (SEQ ID NO: 104), wherein each of B₁-B₈, B₁₁, and U₁-U₉ are independently an amino acid. In embodiments, at least 1, 2, 3, 4, or 5 amino acids of B₁-B₈, B₁₁, and U₁-U₉ do not correspond to the amino acid of a wild-type affimer sequence. In embodiments, at least 1, 2, 3, 4, 5, 6, 7, 8, 9, 10, 11, 12, 13, 14, 15, 16, or 17 amino acids (or combinations thereof) of B₁-B₈ and U₁-U₉ do not correspond to the amino acid of a wild-type affimer sequence. In embodiments, 1, 2, 3, 4, 5, 6, 7, 8, 9, 10, 11, 12, 13, 14, 15, 16, 17, or 18 amino acids (or combinations thereof) of B₁-B₈ and U₁-U₉ do not correspond to the amino acid of a wild-type affimer sequence. In embodiments, provided herein are polypeptides with between 1-5 or between 1-10, or between 1-17 amino acids, e.g., about 1, 2, 3, 4, 5, 6, 7, 8, 9, 10, 11, 12, 13, 14, 15, 16, or 17 amino acids, which are mutated, deleted, or inserted, as compared to the polypeptide of SEQ ID NO: 104. In embodiments, at least 1, 2, 3, 4, or 5 amino acids of B₁-B₈ and U₁-U₉ do not correspond to the amino acid of a wild-type affimer sequence (provided in Table 2A). In embodiments, the mutations, deletions, or insertions may occur at amino acid positions other than B₁-B₈ and U₁-U₉ of the polypeptide of SEQ ID NO: 104.

In embodiments, B₁ is M or V. In embodiments, B₁ is M. In embodiments, B₂ is S or P. In embodiments, B₂ is S. In embodiments, B₃ is G or S. In embodiments, B₃ is G. In embodiments, B₄ is C or G. In embodiments, B₄ is C. In embodiments, B₅ is R or K. In embodiments, B₅ is R. In embodiments, B₆ is G or S. In embodiments, B₆ is S. In embodiments, B₇ is R or C. In embodiments, B₇ is C. In embodiments, B₈ is V or M. In embodiments, B₈ is M. In embodiments, B₁₁ is G or D.

In embodiments, each of U₁-U₉ are independently I, G, A, S, V, W, R, Y, D, K, P, V, K or C. In embodiments, U₁ is I, G, A, S, V, W, or A. In embodiments, U₂ is R, V, G, A, S, I, P, Y, or N. In embodiments, U₃ is A, I, Y, G, R, S, V, D, P, or K. In embodiments, U₄ is I, Y, P, V, D, R, K, S, P, or W. In embodiments, U₅ is V, A, G, Y, R, I, W, P, C or S. In embodiments, U₆ is Y, R, V, W, I, V, S, N, P, or D. In embodiments, U₇ is W, S, Y, V, R, I, A, or P. In embodiments, U₈ is S, Y, K, A, P, W, R, N, or D. In embodiments, U₉ is G, S, A, P, W, R, Y, I, V, or K.

In embodiments, each of U₁-U₉ is independently R, I, V, A, W, S, P, Y, K, or G. In embodiments, U₁ is R, S, or V; U₂ is I, R, P, or W; U₃ is V or W; U₄ is R, Y, or V; U₅ is V or W; U₆ is A, I, P, or S; U₇ is V, R, K, or D; U₈ is W, G, or P; or U₉ is V, G, or A.

In embodiments, each of U₁-U₉ are independently R, I, V, A, and W. In embodiments, U₁ is R. In embodiments, U₂ is I. In embodiments, U₃ is V. In embodiments, 1.14 is R. In embodiments, U₅ is V. In embodiments, U₆ is A. In embodiments, U₇ is V. In embodiments, U₈ is W. In embodiments, U₉ is V.

In embodiments, the polypeptide comprises a sequence selected from Table 2D or a polypeptide with at least 80%, at least 85%, at least 90%, at least 91%, at least 92%, at least 93%, at least 94%, at least 95%, at least 96%, at least 97%, at least 98%, at least 99%, or 100% identity to the sequence of Table 2D.

TABLE 2D

| | SEQ |
|---|---|
| | ID |
| Polypeptide | NO: |
| ATGVRAVSGNENGLEIEELARFAVDEHNKKENALLEFVRVVKAKE | 85 |
| QVVAGTMYYLTLEAKDGGKKKLYEAKVWVKRIVRVAVWVNFRE | |
| LQEFKPVGDA | |
| ATGVRAVPGNENSLEIEELARFAVDEHNKKENALLEFVRVVKAKE | 86 |
| QVVACTMYYLTLEAKDDGKKKLYEAKVWVKSRVRVIRWGNFKE | |
| LQEFKPVGDA | |
| ATGVRAVSGNENSLEIEELARFAVDEHNKKENALLEFVRVVKAKE | 87 |
| QVVAGTMYYLTLEAKDGGKKKLYEAKVWVKSPVYWPKGGNFKEL | |
| QEFKPVGDA | |
| ATGVRAMPGNENSLEIEELARFAVDEHNKKENALLEFVRVVKAKE | 88 |
| QVVAGTMYYLTLEAKDGGKKKLYEAKVWVKVWWVVSDPANFK | |
| ELQEFKPVGDA | |
| ATGVRAVPGNENSLEIEELARFAVDEHNKKENALLEFVRVMKAKE | 60 |
| QVVAGTMYYLTLEAKDGGKKKLYEAKVWVKGVIYAYSYSNFKEL | |
| QEFKPVGDA | |
| ATGVRAVPSNENSLEIEELARFAVDEHNKKENALLEFVRVVKAKE | 67 |
| QVVAGTMYYLTLEAKDGGKKKLYEAKVWVKAIKYVRYKANFKEL | |
| QEFKPVGDA | |
| ATGVRAVPGNENSLEIEELARFAVDEHNKKENALLEFVCVVKAKE | 77 |
| QVVAGTMYYLTLEAKDGGKKKLYEAKVWVKVAPIYSVDSNFKELQ | |
| EFKPVGDA | |

### Affimer—Combination Scaffold

In embodiments, the compositions provided herein comprise polypeptides comprising the sequence ATGVRAB₁B₂GNENB₃LEIEELARFAVDEHNKKENALLEFVRVVKAKEQZ₁Z₂Z₃Z₄Z₃Z₆Z₇Z₈Z₉TMYYLTLEAKDGGKKKLYEAKVWVKU₁U₁₂U₃U₄U₃U₆U₇U₈U₉NFKELQEFKP VGDA, wherein each of B₁, B₂, B₃, Z₁-Z₉ and U₁-U₉ are independently an amino acid (SEQ ID NO: 89), provided that at least 1, 2, 3, 4, or 5 amino acids of B₁-B₃, Z₁-Z₉ and U₁-U₉ do not correspond to the amino acid of a wild-type affimer sequence of Table 2A. In embodiments, at least 1, 2, 3, 4, 5, 6, 7, 8, 9, 10, 11, 12, 13, 14, 15, 16, 17, 18, 19, 20, or 21 amino acids of B₁-B₃, Z₁-Z₉ and U₁-U₉ do not correspond to the amino acid of a wild-type affimer sequence of Table 2A. In embodiments, 1, 2, 3, 4, 5, 6, 7, 8, 9, 10, 11, 12, 13, 14, 15, 16, 17, 18, 19, 20, 21, or 22 amino acids (or ranges therein) of B₁-B₃, Z₁-Z₉ and U₁-U₉ do not correspond to the amino acid of a wild-type affimer sequence of Table 2A. In embodiments, provided herein are polypeptides with between 1-5, between 1-10, or between 1-22 amino acids, e.g., about 1, 2, 3, 4, 5, 6, 7, 8, 9, 10, 11, 12, 13, 14, 15, 16, 17, 18, 19, 20, 21, or 22 amino acids, which are mutated, deleted, or inserted, as compared to the polypeptide of SEQ ID NO: 89. In embodiments, the mutations, deletions, or insertions may occur at amino acid positions other than B₁-B₃, Z₁-Z₉ and U₁-U₉ of the polypeptide of SEQ ID NO: 89. In embodiments, the compositions provided herein comprise polypeptides comprising the sequence ATGVRAB₁B₂B₆NENB₃LEIEELARFAVDEHNKKENALLEFVB₇B₉B₈KAKEQZ₁Z₂Z₃Z₄Z KB₁₀VGDA, wherein each of B₁, B₂, B₃, B₃-B₁₀, Z₁-Z₉ and U₁-U₉ are independently an amino acid (SEQ ID NO: 107), provided that at least 1, 2, 3, 4, or 5 amino acids of B₁-B₃, Z₁-Z₉, and U₁-U₉ do not correspond to the amino acid of a wild-type affimer sequence of Table 2A. In embodiments, provided herein are polypeptides with between 1-5 or between 1-10 amino acids, i.e., about 1, 2, 3, 4, 5, 6, 7, 8, 9, or 10 amino acids, which are mutated, deleted, or inserted, as compared to the polypeptide of SEQ ID NO: 107. In embodiments, the mutations, deletions, or insertions may occur at amino acid positions other than B₁-B₃, Z₁-Z₉ and U₁-U₉ of the polypeptide of SEQ ID NO: 107.

In embodiments, B₁ is M or V. In embodiments, B₁ is M. In embodiments, B₂ is S or P. In embodiments, B₂ is S. In embodiments, B₃ is G or S. In embodiments, B₃ is G. In embodiments, B₅ is R or K. In embodiments, B₅ is R. In embodiments, B₆ is G or S. In embodiments, B₆ is S. In embodiments, B₇ is R or C. In embodiments, B₇ is C. In embodiments, B₈ is V or M. In embodiments, B₉ is V or M. In embodiments, B₁₀ is P or L. In embodiments, B₈ is M. In embodiments, B₁₁ is G or D.

In embodiments, each of U₁-U₉ are independently I, G, A, S, V, W, R, Y, D, K, P, V, K or C. In embodiments, U₁ is I, G, A, S, V, W, or A. In embodiments, U₂ is R, V, G, A, S, I, P, Y, or N. In embodiments, U₃ is A, I, Y, G, R, S, V, D, P, or K. In embodiments, U₄ is I, Y, P, V, D, R, K, S, P, or W. In embodiments, U₅ is V, A, G, Y, R, I, W, P, C or S. In embodiments, U₆ is Y, R, V, W, I, V, S, N, P, or D. In embodiments, U₇ is W, S, Y, V, R, I, A, or P. In embodiments, U₈ is S, Y, K, A, P, W, R, N, or D. In embodiments, U₉ is G, S, A, P, W, R, Y, I, V, or K.

In embodiments, each of U₁-U₉ is independently R, I, V, A, W, S, P, Y, K, or G. In embodiments, U₁ is R, S, or V; U₂ is I, R, P, or W; U₃ is V or W; U₄ is R, Y, or V; U₅ is V or W; U₆ is A, I, P, or S; U₇ is V, R, K, or D; U₈ is W, G, or P; or U₉ is V, G, or A. In embodiments, each of U₁-U₉ is independently Y, G, S, C, W, R, V, I, D, P, or A. In embodiments, U₁ is Y or W; U₂ is G or V; U₃ is G or I; U₄ is S or R; U₅ is C or G; U₆ is W or D; U₇ is R or S; U₈ is G or P; or U₉ is G or A.

In embodiments, each of Z₁-Z₉ is independently Y, I, S, V, D, K, W, A, R, or N. In embodiments, Z₁ is Y or R. In embodiments, Z₂ is I or A. In embodiments, Z₃ is S or R. In embodiments, Z₄ is V or Y. In embodiments, Z₅ is D or Y. In embodiments, Z₆ is K or Y. In embodiments, Z₇ is W or V. In embodiments, Z₈ is V or N. In embodiments, Z₉ is A or R.

### Affibody

In embodiments, the compositions provided herein comprise polypeptides comprising the sequence: VDNKENKEJ₁J₂J₃AJ₄J₅EIJ₆J₇LPNLNJ₈J₉QJ₁₀J₁₁AFIJ₁₂SLJ₁₃DDPJA₁₄J₁₅J₁₆ANLLAEAK KLNDAQAPK (SEQ ID NO: 90), wherein each of J₁-J₁₆ is independently an amino acid. In embodiments, at least 1, 2, 3, 4, or 5 amino acids of J₁-J₁₆ do not correspond to the amino acid of a wild-type affibody sequence. In embodiments, at least 1, 2, 3, 4, 5, 6, 7, 8, 9, 10, 11, 12, 13, 14, or 15, amino acids of J₁-J₁₆ do not correspond to the amino acid of a wild-type affibody sequence. In embodiments, at least 1, 2, 3, 4, 5, 6, 7, 8, 9, 10, 11, 12, 13, 14, 15, or 16 amino acids (or ranges therein) of J₁-J₁₆ do not correspond to the amino acid of a wild-type affibody sequence. In embodiments, the mutations, deletions, or insertions may occur at amino acid positions other than J₁-J₁₆ of the polypeptide of SEQ ID NO: 90. In embodiments, the compositions provided herein comprise polypeptides comprising the sequence: VDNKFNKEJ₁J₂J₃AJ₄J₅EIJ₆J₇LPNLNJ₈J₉QJ₁₀AFIJ₁₂SJ₁₇J₁₃DDPJ₁₄J₁₃J₁₆ANLLAEAK KLNDAQAPK (SEQ ID NO: 108), wherein each of J₁-J₁₇ is independently an amino acid or any one of J₁-J₁₇ is absent. In embodiments, at least 1, 2, 3, 4, or 5 amino acids of J₁-J₁₇ do not correspond to the amino acid of a wild-type affibody sequence. In embodiments, at least 1, 2, 3, 4, 5, 6, 7, 8, 9, 10, 11, 12, 13, 14, 15, or 16 amino acids of J₁-J₁₇ do not correspond to the amino acid of a wild-type affibody sequence. In embodiments, at least 1, 2, 3, 4, 5, 6, 7, 8, 9, 10, 11, 12, 13, 14, 15, 16, or 17 amino acids of J₁-J₁₇ do not correspond to the amino acid of a wild-type affibody sequence. In embodiments, provided herein are polypeptides with between 1-5, between 1-10, or between 1-17 amino acids, e.g., about 1, 2, 3, 4, 5, 6, 7, 8, 9, 10, 11, 12, 13, 14, 15, 16, or 17 amino acids, which are mutated, deleted, or inserted, as compared to the polypeptide of SEQ ID NO: 90. In embodiments, provided herein are polypeptides with between 1-5, between 1-10, or between 1-17 amino acids, i.e., about 1, 2, 3, 4, 5, 6, 7, 8, 9, 10, 11, 12, 13, 14, 15, 16, or 17 amino acids, which are mutated, deleted, or inserted, as compared to the polypeptide of SEQ ID NO: 108. In embodiments, the mutations, deletions, or insertions may occur at amino acid positions other than J₁-J₁₇ of the polypeptide of SEQ ID NO: 90. The wild-type affibody sequence is provided in Table 3A.

TABLE 3A

| Polypeptide | SEQ ID NO: |
|---|---|
| VDNKFNKEQQNAFYEILHL | 9 |
| PNLNEEQRNAFIQSLKDDP | |
| SQSANLLAEAKKLNDAQAP | |
| K | |

In embodiments, each of J₁-J₁₆ are independently Q, W, L, Y, H, A, M, R, E, K, V, S, Q, R, or P. In embodiments, each of J₁-J₃ are independently Q, W, L, Y, A, S, V, E, M, K, V, H, or R. In embodiments, J₁ is Q, W, Y, E, L, M, or S. In embodiments, J₂ is W, Y, S, L, A, M, Q, V, or R. In embodiments, J₃ is L, A, V, M, Y, W, or H. In embodiments, each of J₄ and J₅ is independently W, E, M, H, V, A, Y, L, or S. In embodiments, J₄ is W, E, M, H, V, A, or Y. In embodiments, J₅ is Y, L, M, V, S, or A. In embodiments, each of J₆ or J₇ is H, W, Y, E, L, Q, A, M, R, or S. In embodiments, J₆ is H, W, Y, E, L, Q, A, Y, M, R, or S. In embodiments, J₇ is Y, W, L, R, S, or A. In embodiments, each of J₈ and J₉ are independently A, M, S, L, H, K, Y, Q, or W. In embodiments, J₈ is A, M, S, L, or H. In embodiments, J₉ is M, K, Y, Q, W, or L. In embodiments, each of J₁₀ and J₁₁ are independently H, W, L, M, E, S, A, R, Y, or V. In embodiments, J₁₀ is H, W, L, M, E, S or A. In embodiments, J₁₁ is R, Y, V, A, L, or E. In embodiments, J₁₂ is L, Y, V, E, M, or Q. In embodiments, J₁₃ is Y, E, V, R, W, Q, or H. In embodiments, each of J₁₄-J₁₆ are independently R, P, H, S or Q. In embodiments, J₁₄ is R or S. In embodiments, J₁₅ is R or Q. In embodiments, J₁₆ is R or S or P or H. In embodiments, J₁₇ is L or P.

In embodiments, the polypeptide comprises a sequence selected from Table 3B or a polypeptide with at least 80%, at least 85%, at least 90%, at least 91%, at least 92%, at least 93%, at least 94%, at least 95%, at least 96%, at least 97%, at least 98%, at least 99%, or 100% identity to the sequence of Table 3B.

TABLE 3B

| | SEQ |
|---|---|
| | ID |
| Polypeptide | NO: |
| VDNKFNKEQWLAWYEIHYLPNLNAMQHRAFILSPYDDPSQSANLL | 91 |
| AEAKKLNDAQAPK | |
| VDNKFNKEWYAAELEIWELPNLNMKQWYAFIYSLEDDPSQSANLL | 92 |
| AEAKKLNDAQAPK | |
| VDNKFNKEYSVAWLEIYLLPNLNMMQLRAFIVSLEDDPSQSANLL | 93 |
| AEAKKLNDAQAPK | |
| VDNKFNKEELMAMYEIEYLPNLNAYQMVAFIVSLVDDPSQSANLL | 94 |
| AEAKKLNDAQAPK | |
| VDNKENKELAYAHMEIWYLPNLNSQQWYAFIESLRDDPSQSANLL | 95 |
| AEAKKLNDAQAPK | |
| VDNKFNKEMSYAVLEILRLPNLNLMQLAFIYSLWDDPSQSANLL | 96 |
| AEAKKLNDAQAPK | |
| VDNKFNKEYYYAAVEIQYLPNLNMWQMLAFIVSLYDDPRRPANLL | 97 |
| AEAKKLNDAQAPK | |
| VDNKFNKEMYVAVLEIHSLPNLNLMQLVAFILSLYDDPSQSANLL | 98 |
| AEAKKLNDAQAPK | |
| VDNKFNKEWSWAYSEIHLLPNLNHLQHYAFIMSLQDDPRRHANLL | 99 |
| AEAKKLNDAQAPK | |
| VDNKFNKEYMLAELEIAALPNLNHWQEVAFIVSLVDDPSQSANLL | 100 |
| AEAKKLNDAQAPK | |
| VDNKFNKESQYAYAEIYLLPNLNHWQSVAFIWSLYDDPSQSANLL | 101 |
| AEAKKLNDAQAPK | |
| VDNKFNKEKVHAMLEIRWLPNLNMLQAYAFIQSLHDDPSQSANL | 102 |
| LAEAKKLNDAQAPK | |
| VDNKFNKEERAAYVEIHWLPNLNSKQWEAFIMSLQDDPSQSANLL | 103 |
| AEAKKLNDAQAPK | |

### Multimeric Polypeptides

In embodiments, provided herein are multimeric polypeptides. As used herein, a multimeric polypeptide refers to a polypeptide containing two or more domains. In embodiments, the domains are polypeptides described herein. In embodiments, the multimeric polypeptide comprises one or more domains having an amino acid sequence according to any one of SEQ ID NOS: 54, 58, 81, 84, 89, 90, 104, and 108. In embodiments, the multimeric polypeptide comprises two or more domains having an amino acid sequence independently selected from any one of SEQ ID NOS: 54, 58, 81, 84, 89, 90, 104, and 108, wherein the two domains are the same. In embodiments, the multimeric polypeptide comprises two or more domains having an amino acid sequence independently selected from any one of SEQ ID NOS: 54, 58, 81, 84, 89, 90, 104, and 108, wherein the two domains are different. In embodiments, the domain is a linker. In embodiments, linker domains separate polypeptide domains having an amino acid sequence independently selected from any one of SEQ ID NOS: 54, 58, 81, 84, 89, 90, 104, and 108. In embodiments, the linker is a polypeptide. In embodiments, the polypeptide linker comprises glycine and serine. In embodiments, the polypeptide linker comprises glycine, serine, and arginine. In embodiments, the polypeptide linker has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100 identity to any one of SEQ ID NOS: 109-168. In embodiments, the multimeric polypeptide comprises two or more different polypeptide linkers. In embodiments, the multimeric polypeptide comprises two or more of the same polypeptide linkers. In embodiments, the multimeric polypeptide comprises one or more domains having an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOS: 54, 58, 81, 84, 89, 90, 104, and 108. In embodiments, the multimeric polypeptide comprises from about 2 to about 100 domains. In embodiments the multimeric polypeptide comprises from about 1 to about 100 domains having an amino acid sequence independently selected from any one of SEQ ID NOS: 54, 58, 81, 84, 89, 90, 104, and 108 or a polypeptide with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOS: 54, 58, 81, 84, 89, 90, 104, and 108. In embodiments the multimeric polypeptide comprises from about 1 to about 100 domains, wherein the domains are linkers. In embodiments, the linker domains have an amino acid sequence independently selected from any one of SEQ ID NOS: 109-168 or an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOS: 109-168. In embodiments, the multimeric polypeptide comprises the linker domain GS G. In embodiments, the multimeric polypeptide comprises one or more linker domains with an amino acid sequence that is at least %, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 109. In embodiments, the multimeric polypeptide described herein comprises about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 domains. In embodiments, the multimeric polypeptide comprises from 2 to about 5, from 3 to about 6, from about 2 to about 8, from about 2 to about 10, from 4 to about 10, from 5 to about 15, from about 13 to about 20, from about 18 to about 25, from about 20 to about 30, from about 25 to about 35, from about 30 to about 40, from about 35 to about 45, or from about 40 to about 50 domains. In embodiments, the C-terminus of the multimeric polypeptide comprises the dipeptide glycine tyrosine. In embodiments, the multimeric polypeptide has an amino acid sequence selected from any one of SEQ ID NOS: 169-193 or an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOS: 169-193.

### Modifications to Polypeptides Described Herein

In embodiments, the polypeptides described herein comprise an N-terminal methionine. In embodiments, the polypeptides described herein lack an N-terminal methionine. In embodiments, the polypeptides described herein comprise a polyhistidine tag. In embodiments, the polyhistidine tag is located at the N-terminus of the polypeptide sequence. In embodiments, the polyhistidine tag is located at the C-terminus of the polypeptide sequence. In embodiments, the polypeptides are biotinylated. In embodiments, the polypeptides described herein comprise the dipeptide, glycine-tyrosine, at the C-terminus.

In embodiments, the polypeptides described are extended at the N- or C-terminus. In embodiments, the extension comprises a polypeptide linker. In embodiments, the linker is selected from the group consisting of: GGGGSGGSGGGGSGGRGS (SEQ ID NO: 109), AAAAA (SEQ ID NO: 110), AAAAAAAAAA (SEQ ID NO: 111), AAAAAAGGGGG (SEQ ID NO: 112), AGAGA (SEQ ID NO: 113), EAAAK (SEQ ID NO: 114), EAAAKEAAAK (SEQ ID NO: 115), EKAREK (SEQ ID NO: 116), GAAGA (SEQ ID NO: 117), GASGAS (SEQ ID NO: 118), GEGEF (SEQ ID NO: 119), GGGGS (SEQ ID NO: 120), GGGGSGGGG (SEQ ID NO: 121), GGGGSGGGGS (SEQ ID NO: 122), GGGGSGGGGSGGGG (SEQ ID NO: 123), GGGGSGGGGSGGGGS (SEQ ID NO: 124), GGGGSGGGGSGGGGSGGGGS (SEQ ID NO: 125), GGGGSGGGGSGGGGSGGGGSGGGGS (SEQ ID NO: 126), GGGS (SEQ ID NO: 127), GGGSGGGS (SEQ ID NO: 128), GGGSGGGSGGG (SEQ ID NO: 129), GGGSGGGSGGGS (SEQ ID NO: 130), GGS, GGSGG (SEQ ID NO: 131), GGSGGS (SEQ ID NO: 132), GGSGGSGGS (SEQ ID NO: 133), GGSGGSGGSGGS (SEQ ID NO: 134), GPGGPG (SEQ ID NO: 135), GPGPG (SEQ ID NO: 136), GPGPGP (SEQ ID NO: 137), GPPGPP (SEQ ID NO: 138), GPSGPG (SEQ ID NO: 139), GPSGPS (SEQ ID NO: 140), GPSGSGPG (SEQ ID NO: 141), GSGGGGSGGGGSGGGGSGS (SEQ ID NO: 142), GSGGGGSGGGGSGS (SEQ ID NO: 143), GSGGGGSGS (SEQ ID NO: 144), GSGGGSGGGSGGGSGS (SEQ ID NO: 145), GSGGGSGGGSGS (SEQ ID NO: 146), GSGGGSGS (SEQ ID NO: 147), GSGGSG (SEQ ID NO: 148), GSGS (SEQ ID NO: 149), GSGSAGSGSA (SEQ ID NO: 150), GSGSGS (SEQ ID NO: 151), GSGSGSGSG (SEQ ID NO: 152), GSSG (SEQ ID NO: 153), GSSGGGGS (SEQ ID NO: 154), KKKKKGGGGG (SEQ ID NO: 155), PAPAP (SEQ ID NO: 156), PGPGPGPGP (SEQ ID NO: 157), QPGSGP (SEQ ID NO: 158), SGAGSAGA (SEQ ID NO: 159), SGGSGG (SEQ ID NO: 160), SGGSGGSGG (SEQ ID NO: 161), SGGSGGSGGSGG (SEQ ID NO: 162), SGSGGSGGSGGSGG (SEQ ID NO: 163), SGSGSGSG (SEQ ID NO: 164), SSGSSGSS (SEQ ID NO: 165), TGTGGSGSGTG (SEQ ID NO: 166), TGTGGTGTGG (SEQ ID NO: 167), and YPKYVKQNTLKLAT (SEQ ID NO: 168). In embodiments, the polypeptides comprise a linker with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOS: 109-168. In embodiments, the linker has the amino acid sequence of SEQ ID NO: 109.

### Methods Utilizing Compositions Comprising AAV Binding Polypeptides

In embodiments, provided herein are methods of binding AAV capsid, comprising contacting AAV capsids with a polypeptide or multimeric polypeptide described herein. In embodiments, the polypeptides described herein bind to a wildtype or mutant AAV particle of any one of the following serotypes: AAV1, AAV2, AAV3 (including types 3A and 3B), AAV4, AAV5, AAV6, AAV7, AAV8, AAV9, AAV10, AAV11, AAV12, AAV13, AAVrh32.33, AAVrh8, AAVrh10, AAVrh74, AAVhu.68, avian AAV, bovine AAV, canine AAV, equine AAV, ovine AAV, snake AAV, bearded dragon AAV, AAV2i8, AAV2g9, AAV-LK03, AAV7m8, AAV Anc80, or AAV PHP.B. In embodiments, the AAV is AAV9. In embodiments, the polypeptides bind to full AAV capsids.

Provided herein is a method of separating full AAV capsids from empty AAV capsids, the method comprising: (a) contacting a polypeptide with a solution comprising the mixture of full AAV capsids and empty AAV capsids, wherein the polypeptide shows at least a two-fold increase in binding preference for full AAV capsids versus empty AAV capsids; (b) allowing the polypeptide to bind the full AAV capsids; (c) separating empty AAV capsids from the full AAV capsids bound to the polypeptide. In embodiments, the polypeptide is any polypeptide described herein. In embodiments, the polypeptide comprises an avimer, affimer, or affibody scaffold sequence, as shown in Table A. In embodiments, the method comprises quantifying full AAV capsids in the sample. In embodiments, the method comprises determining a ratio of full AAV capsids to empty AAV capsids in the sample. In embodiments, the method comprises quantifying the full virus particles in the sample. In embodiments, the solution is step (a) comprises a cell lysate. In embodiments, the solution in step (a) is clarified.

In embodiments, the polypeptides described herein have a full capsid bias (“FCBI”). The FCBI refers to the ability of the polypeptides to preferentially bind to full AAV capsids. The FCBI is calculated using the following equation: ((AAV Full Capsids RLU of Sample)/(AAV Full Capsids RLU of Control))/((AAV Empty Capsids RLU of Sample)/(AAV Empty Capsids RLU of Control)). The RLU refers to the relative luminescence intensity. In embodiments, the FCBI is greater than 1.5, greater than 2, greater than 2.5, greater than 3, greater than 3.5, greater than 4, greater than 4.5, greater than 5, greater than 5.5, greater than 6, greater than 6.5, greater than 7, greater than 7.5, greater than 8, greater than 8.5, greater than 9, greater than 9.5, greater than 10, greater than 10.5, greater than 11, greater than 11.5, greater than 12, greater than 12.5, greater than 13, greater than 13.5, greater than 14, greater than 14.5, greater than 15, greater than 15.5, greater than 16, greater than 16.5, greater than 17, greater than 17.5, greater than 18, greater than 18, greater than 18.5, greater than 19, greater than 19.5, or greater than 20. In embodiments, the FCBI is greater than 17. In embodiments, the FCBI ranges from 1.5 to about 20, including all subranges and ranges therebetween. For example, the FCBI may be about 1.5, about 2, about 2.5, about 3, about 3.5, about 4, about 4.5, about 5, about 5.5, about 6, about 6.5, about 7, about 7.5, about 8, about 8.5, about 9, about 9.5, about about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, or about 20.

In embodiments, each polypeptide described herein has an AAV specificity ratio (AAV SR). The AAV SR is calculated using the following equation: AAV SR=(AAV full capsids RLU)/(control polypeptide RLU). In embodiments, the AAV SR refers to preferential binding of the polypeptides compared to a control polypeptide. In embodiments, the control polypeptide is human serum albumin. In embodiments, the AAV SR is greater than about 1, greater than about 2, greater than about 3, greater than about 4, greater than about 5, greater than about 6, greater than about 7, greater than about 8, greater than about 9, greater than about greater than about 11, greater than about 12, greater than about 13, greater than about 14, greater than about 15, greater than about 16, greater than about 17, greater than about 18, greater than about 19, greater than about 20, greater than about 21, greater than about 22, greater than about 23, greater than about 24, greater than about 25, greater than about 26, greater than about 27, greater than about 28, greater than about 29, greater than about 30, greater than about 31, greater than about 32, greater than about 33, greater than about 34, greater than about 35, greater than about 36, greater than about 37, greater than about 38, greater than about 39, greater than about 40, greater than about 41, greater than about 42, greater than about 43, greater than about 44, greater than about 45, greater than about 46, greater than about 47, greater than about 48, greater than about 49, greater than about 50, greater than about 51, greater than about 52, greater than about 53, greater than about 54, greater than about 55, greater than about 56, greater than about 57, greater than about 58, greater than about 59, greater than about 60, greater than about 61, greater than about 62, greater than about 63, greater than about 64, greater than about 65, greater than about 66, greater than about 67, greater than about 68, greater than about 69, greater than about 70, greater than about 71, greater than about 72, greater than about 73, greater than about 74, greater than about 75, greater than about 76, greater than about 77, greater than about 78, greater than about 79, greater than about 80, greater than about 81, greater than about 82, greater than about 83, greater than about 84, greater than about 85, greater than about 86, greater than about 87, greater than about 88, greater than about 89, greater than about 90, greater than about 91, greater than about 92, greater than about 93, greater than about 94, greater than about 95, greater than about 96, greater than about 97, greater than about 98, greater than about 99, greater than about 100. In embodiments, the AAV SR ranges from 1 to about 100, including all ranges and subranges therebetween. In embodiments, the AAV SR is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100. In embodiments, the AAV SR is at least 2.5. In embodiments, the AAV SR is at least 5. In embodiments, the AAV SR is at least 15. In embodiments, the AAV SR is at least 85.

In embodiments, the methods provided herein may be used during manufacturing of AAV particles. In embodiments, the methods are used during quality control to quantify the number of particles having full AAV capsids. In embodiments, the methods described herein may be used during purification of AAV particles. In embodiments, the polypeptides are used during ELISA methods. In embodiments, the methods are used after lysis or after clarification.

### EXAMPLES

### Example 1. Development of AAV-Binding Polypeptides

Purpose: Four protein scaffold libraries were panned to develop novel polypeptides that bind to AAV particles and capsids. The protein scaffold sequences used for each library are identified in Table A1 below. Table A2 contains amino acids included at positions X₁-X₂₉ for the avimer. Table A3 contains amino acids included at positions U₁-U₉ for the Affimer Loop 1. Table A4 contains amino acids included at positions Z₁-Z₉ and U₁-U₉ for the Affimer Loop 2. Table A5 contains amino acids included at positions B₁-B₄ and U₁-U₉ for the Affimer Loop 2 with additional mutations. Table A6 contains amino acids included at positions J₁-J₁₆ for the affibody. The scaffold sequences are based on the reference polypeptide sequences of Table B.

TABLE A1
Protein Scaffold Libraries

| | | SEQ |
|---|---|---|
| | | ID |
| Scaffold | Sequence | NO: |
| Avimer | CX₁X₂X₃X₄FX₅CX₆X₇X₈X₉X₁₀CX₁₁X₁₂X₁₃TW | 1 |
| | VCDGX₁₄X₁₅DCX₁₆DX₁₇X₁₈DEX₁₉X₂₀CTPTP | |
| Affimer | ATGVRAVPGNENSLEIEELARFAVDEHNKK | 2 |
| Loop 1 | ENALLEFVRVVKAKEQVVAGTMYYLTLEAK | |
| | DGGKKKLYEAKVWVKU₁U₂U₃U₄U₅U₆U₇U₈U₉ | |
| | NFKELQEFKPVGDA | |
| Affimer | ATGVRAVPGNENSLEIEELARFAVDEHNKKE | 3 |
| Loop 2 | NALLEFVRVVKAKEQZ₁Z₂Z₃Z₄Z₅Z₆Z₇Z₈Z₉T | |
| | MYYLTLEAKDGGKKKLYEAKVWVKU₁U₂U₃U₄ | |
| | U₅U₆U₇U₈U₉NFKELQEFKPVGDA | |
| Affimer | ATGVRAB₁B₂GNENB₃LEIEELARFAVDEHNK | 4 |
| Loop 2 | KENALLEFVRVVKAKEQVVAB₄TMYYLTLEA | |
| with | KDGGKKKLYEAKVWVKU₁U₂U₃U₄U₅U₆U₇U₈U₉ | |
| additional | NFKELQEFKPVGDA | |
| mutations | | |
| Affibody | VDNKFNKEJ₁J₂J₃AJ₄J₅EIJ₆J₇LPNLNJ₈J₉ | 5 |
| | QJ₁₀J₁₁AFIJ₁₂SLJ₁₃DDPJ₁₄J₁₅J₁₆ANLLAE | |
| | AKKLNDAQAPK | |

TABLE A2
Avimer: Potential Amino Acids at X₁-X₂₀

| Position | Amino Acids in the Library |
|---|---|
| X₁ | Gly, Ala, Val, Ile, Pro, Asp, Arg, |
| | Lys, Tyr, Trp, Asn, Ser |
| X₂ | Gly, Ala, Val, Ile, Pro, Asp, Arg, |
| | Lys, Tyr, Trp, Asn, Ser |
| X₃ | Gly, Ala, Val, Ile, Pro, Asp, Arg, |
| | Lys, Tyr, Trp, Asn, Ser |
| X₄ | Gly, Ala, Val, Ile, Pro, Asp, Arg, |
| | Lys, Tyr, Trp, Asn, Ser |
| X₅ | Gly, Ala, Val, Ile, Pro, Asp, Arg, |
| | Lys, Tyr, Trp, Asn, Ser |
| X₆ | Gly, Ala, Val, Ile, Pro, Asp, Arg, |
| | Lys, Tyr, Trp, Asn, Ser |
| X₇ | Gly, Ala, Val, Ile, Pro, Asp, Arg, |
| | Lys, Tyr, Trp, Asn, Ser |
| X₈ | Gly, Ala, Val, Ile, Pro, Asp, Arg, |
| | Lys, Tyr, Trp, Asn, Ser |
| X₉ | Gly, Ala, Val, Ile, Pro, Asp, Arg, |
| | Lys, Tyr, Trp, Asn, Ser |
| X₁₀ | Gly, Ala, Val, Ile, Pro, Asp, Arg, |
| | Lys, Tyr, Trp, Asn, Ser |
| X₁₁ | Gly, Ala, Val, Ile, Pro, Asp, Arg, |
| | Lys, Tyr, Trp, Asn, Ser |
| X₁₂ | Gly, Ala, Val, Ile, Pro, Asp, Arg, |
| | Lys, Tyr, Trp, Asn, Ser |
| X₁₃ | Gly, Ala, Val, Ile, Pro, Asp, Arg, |
| | Lys, Tyr, Trp, Asn, Ser |
| X₁₄ | Gly, Ala, Val, Ile, Pro, Asp, Arg, |
| | Lys, Tyr, Trp, Asn, Ser |
| X₁₅ | Gly, Ala, Val, Ile, Pro, Asp, Arg, |
| | Lys, Tyr, Trp, Asn, Ser |
| X₁₆ | Gly, Ala, Val, Ile, Pro, Asp, Arg, |
| | Lys, Tyr, Trp, Asn, Ser |
| X₁₇ | Gly, Ala, Val, Ile, Pro, Asp, Arg, |
| | Lys, Tyr, Trp, Asn, Ser |
| X₁₈ | Gly, Ala, Val, Ile, Pro, Asp, Arg, |
| | Lys, Tyr, Trp, Asn, Ser |
| X₁₉ | Gly, Ala, Val, Ile, Pro, Asp, Arg, |
| | Lys, Tyr, Trp, Asn, Ser |
| X₂₀ | Gly, Ala, Val, Ile, Pro, Asp, Arg, |
| | Lys, Tyr, Trp, Asn, Ser |

TABLE A3
Affimer Loop 1: Potential Amino Acids at U₁-U₉

| Position | Amino Acids in the Library |
|---|---|
| U₁ | Gly, Ala, Val, Ile, Pro, Asp, Arg, |
| | Lys, Tyr, Trp, Asn, Ser |
| U₂ | Gly, Ala, Val, Ile, Pro, Asp, Arg, |
| | Lys, Tyr, Trp, Asn, Ser |
| U₃ | Gly, Ala, Val, Ile, Pro, Asp, Arg, |
| | Lys, Tyr, Trp, Asn, Ser |
| U₄ | Gly, Ala, Val, Ile, Pro, Asp, Arg, |
| | Lys, Tyr, Trp, Asn, Ser |
| U₅ | Gly, Ala, Val, Ile, Pro, Asp, Arg, |
| | Lys, Tyr, Trp, Asn, Ser |
| U₆ | Gly, Ala, Val, Ile, Pro, Asp, Arg, |
| | Lys, Tyr, Trp, Asn, Ser |
| U₇ | Gly, Ala, Val, Ile, Pro, Asp, Arg, |
| | Lys, Tyr, Trp, Asn, Ser |
| U₈ | Gly, Ala, Val, Ile, Pro, Asp, Arg, |
| | Lys, Tyr, Trp, Asn, Ser |
| U₉ | Gly, Ala, Val, Ile, Pro, Asp, Arg, |
| | Lys, Tyr, Trp, Asn, Ser |

TABLE A4
Affimer Loop 2: Potential Amino Acids at Z₁-Z₉ and U₁-U₉

| Position | Amino Acids in the Library |
|---|---|
| Z₁ | Ala, Asp, Gly, Ile, Lys, Asn, Pro, Arg, |
| | Ser, Val, Trp, Tyr |
| Z₂ | Ala, Asp, Gly, Ile, Lys, Asn, Pro, Arg, |
| | Ser, Val, Trp, Tyr |
| Z₃ | Ala, Asp, Gly, Ile, Lys, Asn, Pro, Arg, |
| | Ser, Val, Trp, Tyr |
| Z₄ | Ala, Asp, Gly, Ile, Lys, Asn, Pro, Arg, |
| | Ser, Val, Trp, Tyr |
| Z₅ | Ala, Asp, Gly, Ile, Lys, Asn, Pro, Arg, |
| | Ser, Val, Trp, Tyr |
| Z₆ | Ala, Asp, Gly, Ile, Lys, Asn, Pro, Arg, |
| | Ser, Val, Trp, Tyr |
| Z₇ | Ala, Asp, Gly, Ile, Lys, Asn, Pro, Arg, |
| | Ser, Val, Trp, Tyr |
| Z₈ | Ala, Asp, Gly, Ile, Lys, Asn, Pro, Arg, |
| | Ser, Val, Trp, Tyr |
| Z₉ | Ala, Asp, Gly, Ile, Lys, Asn, Pro, Arg, |
| | Ser, Val, Trp, Tyr |
| U₁ | Gly, Ala, Val, Ile, Pro, Asp, Arg, Lys, |
| | Tyr, Trp, Asn, Ser |
| U₂ | Gly, Ala, Val, Ile, Pro, Asp, Arg, Lys, |
| | Tyr, Trp, Asn, Ser |
| U₃ | Gly, Ala, Val, Ile, Pro, Asp, Arg, Lys, |
| | Tyr, Trp, Asn, Ser |
| U₄ | Gly, Ala, Val, Ile, Pro, Asp, Arg, Lys, |
| | Tyr, Trp, Asn, Ser |
| U₅ | Gly, Ala, Val, Ile, Pro, Asp, Arg, Lys, |
| | Tyr, Trp, Asn, Ser |
| U₆ | Gly, Ala, Val, Ile, Pro, Asp, Arg, Lys, |
| | Tyr, Trp, Asn, Ser |
| U₇ | Gly, Ala, Val, Ile, Pro, Asp, Arg, Lys, |
| | Tyr, Trp, Asn, Ser |
| U₈ | Gly, Ala, Val, Ile, Pro, Asp, Arg, Lys, |
| | Tyr, Trp, Asn, Ser |
| U₉ | Gly, Ala, Val, Ile, Pro, Asp, Arg, Lys, |
| | Tyr, Trp, Asn, Ser |

TABLE A5
Affimer Loop 2: Potential Amino Acids at B₁-B₄, U₁-U₉

| Position | Amino Acids in the Library |
|---|---|
| B₁ | Ala, Asp, Gly, Ile, Lys, Asn, Pro, Arg, |
| | Ser, Val, Trp, Tyr |
| B₂ | Ala, Asp, Gly, Ile, Lys, Asn, Pro, Arg, |
| | Ser, Val, Trp, Tyr |
| B₃ | Ala, Asp, Gly, Ile, Lys, Asn, Pro, Arg, |
| | Ser, Val, Trp, Tyr |
| B₄ | Ala, Asp, Gly, Ile, Lys, Asn, Pro, Arg, |
| | Ser, Val, Trp, Tyr |
| U₁ | Gly, Ala, Val, Ile, Pro, Asp, Arg, Lys, |
| | Tyr, Trp, Asn, Ser |
| U₂ | Gly, Ala, Val, Ile, Pro, Asp, Arg, Lys, |
| | Tyr, Trp, Asn, Ser |
| U₃ | Gly, Ala, Val, Ile, Pro, Asp, Arg, Lys, |
| | Tyr, Trp, Asn, Ser |
| U₄ | Gly, Ala, Val, Ile, Pro, Asp, Arg, Lys, |
| | Tyr, Trp, Asn, Ser |
| U₅ | Gly, Ala, Val, Ile, Pro, Asp, Arg, Lys, |
| | Tyr, Trp, Asn, Ser |
| U₆ | Gly, Ala, Val, Ile, Pro, Asp, Arg, Lys, |
| | Tyr, Trp, Asn, Ser |
| U₇ | Gly, Ala, Val, Ile, Pro, Asp, Arg, Lys, |
| | Tyr, Trp, Asn, Ser |
| U₈ | Gly, Ala, Val, Ile, Pro, Asp, Arg, Lys, |
| | Tyr, Trp, Asn, Ser |
| U₉ | Gly, Ala, Val, Ile, Pro, Asp, Arg, Lys, |
| | Tyr, Trp, Asn, Ser |

TABLE A6
Affibody: Potential Amino Acids at J₁-J₁₆

| Position | Amino Acids in the Library |
|---|---|
| J₁ | Met, Ala, Arg, Lys, Leu, Glu, Gln, Trp, |
| | Tyr, His, Val, Ser |
| J₂ | Met, Ala, Arg, Lys, Leu, Glu, Gln, Trp, |
| | Tyr, His, Val, Ser |
| J₃ | Met, Ala, Arg, Lys, Leu, Glu, Gln, Trp, |
| | Tyr, His, Val |
| J₄ | Met, Ala, Arg, Lys, Leu, Glu, Gln, Trp, |
| | Tyr, His, Val, Ser |
| J₅ | Met, Ala, Arg, Lys, Leu, Glu, Gln, Trp, |
| | Tyr, His, Val, Ser |
| J₆ | Met, Ala, Arg, Lys, Leu, Glu, Gln, Trp, |
| | Tyr, His, Val |
| J₇ | Met, Ala, Arg, Lys, Leu, Glu, Gln, Trp, |
| | Tyr, His, Ser |
| J₈ | Met, Ala, Arg, Lys, Leu, Glu, Gln, Trp, |
| | Tyr, His, Ser |
| J₉ | Met, Ala, Arg, Lys, Leu, Glu, Gln, Trp, |
| | Tyr, His, Ser |
| J₁₀ | Met, Ala, Arg, Lys, Leu, Glu, Gln, Trp, |
| | Tyr, His, Val, Ser |
| J₁₁ | Met, Ala, Arg, Lys, Leu, Glu, Gln, Trp, |
| | Tyr, His, Val |
| J₁₂ | Met, Ala, Arg, Lys, Leu, Glu, Gln, Trp, |
| | Tyr, His, Val, Ser |
| J₁₃ | Met, Ala, Arg, Lys, Leu, Glu, Gln, Trp, |
| | Tyr, His, Val, Ser |
| J₁₄ | Ser, Arg |
| J₁₅ | Gln, Arg |
| J₁₆ | Ser, His, Pro |

TABLE B
Wild-Type Polypeptide Sequences

| Refer- | | | |
|---|---|---|---|
| ence | | Basis | SEQ |
| Se- | | for | ID |
| quence | Sequence | Scaffold | NO: |
| 1 | TCSQDEFRCHDGK CISRQFVCD | Avimer | 6 |
| | SDRDCLDGSDEASCP | | |
| 2 | TCKSGDFSCGGRVNRCIPQFWRC | Avimer | 7 |
| | DGQVDCDNGSDEQG | | |
| 3 | ATGVRAVPGNENSLEIEELARFAV | Affimer | 8 |
| | DEHNKKENALLEFVRVVKAKEQVV | Loop | |
| | AGTMYYLTLEAKDGGKKKLYEAKV | 1, | |
| | WVKPWENFKELQEFKPVGDA | Affimer | |
| | | Loop | |
| | | 2, and | |
| | | Affimer | |
| | | Loop | |
| | | 2 with | |
| | | additional | |
| | | mutations | |
| 4 | VDNKFNKEQQNAFYEILHLPN | Affibody | 9 |
| | LNEEQRNAFIQSLKDDPSQS | | |
| | ANLLAEAKKLNDAQAPK | | |

Methods: A library encoding the protein scaffold sequences of Table A was produced using a trimer-controlled synthesis method. The trimer-controlled library specifies an exact combination of codons and a precise percentage of desired amino acids.

88 variants from each scaffold library cloned from the Round 5 panning pools were sequenced. Of the 264 variants sequenced, 48 Avimer, 47 Affibody, and 52 Affimer variants were free of frameshift causing mutations. Lysates of bacterial expression for these 147 variants that passed the Sanger sequencing screening were evaluated by ELISA to measure binding specifically to AAV9 capsids coated on the assay plate. Some variants contained spontaneous mutations compared to the polypeptides of SEQ ID NOS: 1-5.

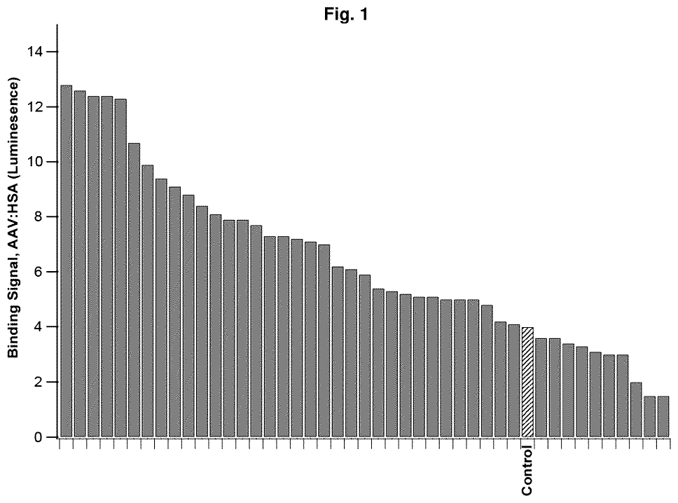

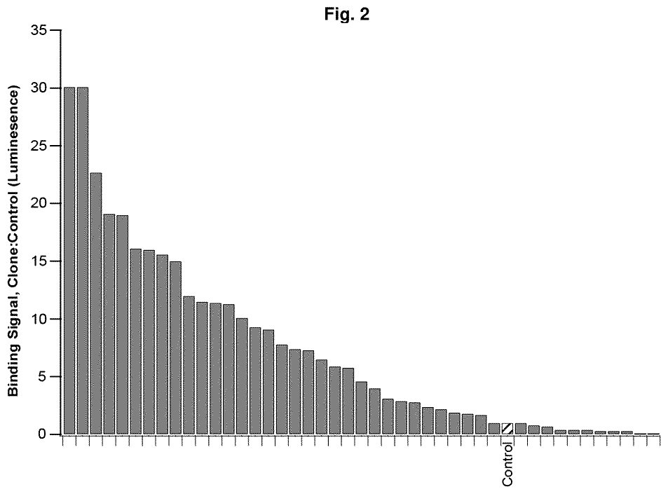

Briefly, AAV9 capsids or human serum albumin (HSA) was coated to 96-well plates by overnight incubation at 4° C. degrees and then blocked. E. coli expression cultures for the selected His-tagged variants were lysed, applied to the AAV9-coated plates, and washed with TBST buffer. Binding was measured by reading luminescence intensity (Relative Luminescence Unit or RLU) on a Biotek Synergy II plate reader after adding horseradish peroxidase labelled anti-his tag to each well. The signal of each variant binding to AAV9 was normalized to the signal generated from either non-specific binding to HSA (FIG. 1) or specific binding to a known AAV9 binding domain control (FIG. 2).

Results: Table C shows polypeptides that exhibit either (A)>3-fold specificity for AAV9 capsids than human serum albumin (HSA) (FIG. 1) or (B) polypeptides that have a >3-fold specificity for AAV9 capsids compared to a human polycystic kidney domain-2 (positive control) by ELISA (FIG. 2). The PKD-2 domain has known affinity to AAV9 capsids, whereas HSA does not bind to AAV9 capsids.

TABLE C
Polypeptides with Specificity for AAV9 Capsids

| | SEQ |
|---|---|
| | ID |
| Polypeptide | NO: |
| (M)CDIPPFVCWWPSWCVWATWVCDGAPDCIDNYDEWICTPTP | 10 |
| (M)CWVKIFRCNIVGVCIVNTWVCDGRVDCSDPPDEGWCTPTP | 11 |
| (M)CAIDNFPCNWYIDCIGATWVCDGIIDCIDIADEYDCTPTP | 12 |
| (M)VDNKFNKEQWLAWYEIHYLPNLNAMQHRAFILSPYDDPSQSANLLA | 13 |
| EAKKLNDAQAPK | |
| (M)VDNKFNKEWYAAELEIWELPNLNMKQWYAFIYSLEDDPSQSANLLA | 14 |
| EAKKLNDAQAPK | |
| (M)VDNKFNKEYSVAWLEIYLLPNLNMMQLRAFIVSLEDDPSQSANLLAE | 15 |
| AKKLNDAQAPK | |
| (M)VDNKFNKEELMAMYEIEYLPNLNAYQMVAFIVSLVDDPSQSANLLA | 16 |
| EAKKLNDAQAPK | |
| (M)VDNKFNKELAYAHMEIWYLPNLNSQQWYAFIESLRDDPSQSANLLA | 17 |
| EAKKLNDAQAPK | |
| (M)VDNKFNKEMSYAVLEILRLPNLNLMQLAFIYSLWDDPSQSANLLAEA | 18 |
| KKLNDAQAPK | |
| (M)VDNKFNKEYYYAAVEIQYLPNLNMWQMLAFIVSLYDDPRRPANLLA | 19 |
| EAKKLNDAQAPK | |
| (M)VDNKFNKEMYVAVLEIHSLPNLNLMQLVAFILSLYDDPSQSANLLAE | 20 |
| AKKLNDAQAPK | |
| (M)VDNKFNKEWSWAYSEIHLLPNLNHLQHYAFIMSLQDDPRRHANLLA | 21 |
| EAKKLNDAQAPK | |
| (M)VDNKFNKEYMLAELEIAALPNLNHWQEVAFIVSLVDDPSQSANLLAE | 22 |
| AKKLNDAQAPK | |
| (M)VDNKFNKESQYAYAEIYLLPNLNHWQSVAFIWSLYDDPSQSANLLAE | 23 |
| AKKLNDAQAPK | |
| (M)VDNKFNKEKVHAMLEIRWLPNLNMLQAYAFIQSLHDDPSQSANLLA | 24 |
| EAKKLNDAQAPK | |
| (M)VDNKFNKEERAAYVEIHWLPNLNSKQWEAFIMSLQDDPSQSANLLA | 25 |
| EAKKLNDAQAPK | |
| (M)ATGVRAVPGNENSLEIEELARFAVDEHNKKENALLEFVRVVKAKEQV | 26 |
| VAGTMYYLTLEAKDGGKKKLYEAKVWVKIRAIVYWSGNFKELQEFKPV | |
| GDA | |
| (M)ATGVRAVPGNENSLEIEELARFAVDEHNKKENALLEFVRVMKAKEQV | 27 |
| VAGTMYYLTLEAKDGGKKKLYEAKVWVKGVIYAYSYSNFKELQEFKPV | |
| GDA | |
| (M)ATGVRAVPGNENSLEIEELARFAVDEHNKKENALLEFVRVVKAKEQV | 28 |
| VAGTMYYLTLEAKDGGKKKLYEAKVWVKAGYIGRSKANFKELQEFKP | |
| VGDA | |
| (M)ATGVRAVPGNENSLEIEELARFAVDEHNKKENALLEFVRVVKAKEQV | 29 |
| VAGTMYYLTLEAKDGGKKKLYEAKVWVKAAGYYVYAPNFKELQEFKP | |
| VGDA | |
| (M)ATGVRAVSGNENGLEIEELARFAVDEHNKKENALLEFVRVVKAKEQV | 30 |
| VAGTMYYLTLEAKDGGKKKLYEAKVWVKRIVRVAVWVNFRELQEFKP | |
| VGDA | |
| (M)ATGVRAVPGNENSLEIEELARFAVDEHNKKENALLEFVRVVKAKEQV | 31 |
| VAGTMYYLTLEAKDGGKKKLYEAKVWVKISRPGWWPWNFKELQEFKP | |
| VGDA | |
| (M)ATGVRAVPGNENSLEIEELARFAVDEHNKKENALLEFVRVVKAKEQV | 32 |
| VAGTMYYLTLEAKDGGKKKLYEAKVWVKSRSVRIWYSNFKELQEFKPV | |
| GDA | |
| (M)ATGVRAVPGNENSLEIEELARFAVDEHNKKENALLEFVRVVKAKEQV | 33 |
| VAGTMYYLTLEAKDGGKKKLYEAKVWVKVRRDIVVPRNFKELQEFKPV | |
| GDA | |
| (M)ATGVRAVPGNENSLEIEELARFAVDEHNKKENALLEFVRVVKAKEQV | 34 |
| VACTMYYLTLEAKDDGKKKLYEAKVWVKSRVRVIRWGNFKELQEFKP | |
| VGDA | |
| (M)ATGVRAVPGNENSLEIEELARFAVDEHNKKENALLEFVRVVKAKEQV | 35 |
| VAGTMYYLTLEAKDGGKKKLYEAKVWVKVRDIVSIRGNFKELQEFKPV | |
| GDA | |
| (M)ATGVRAVPSNENSLEIEELARFAVDEHNKKENALLEFVRVVKAKEQV | 36 |
| VAGTMYYLTLEAKDGGKKKLYEAKVWVKAIKYVRYKANFKELQEFKP | |
| VGDA | |
| (M)ATGVRAVSGNENSLEIEELARFAVDEHNKKENALLEFVRVVKAKEQV | 37 |
| VAGTMYYLTLEAKDGGKKKLYEAKVWVKSPVYWPKGGNFKELQEFKP | |
| VGDA | |
| (M)ATGVRAVPGNENSLEIEELARFAVDEHNKKENALLEFVRVVKAKEQV | 38 |
| VAGTMYYLTLEAKDGGKKKLYEAKVWVKSASYYVYSYNFKELQEFKPV | |
| GDA | |
| (M)ATGVRAVPGNENSLEIEELARFAVDEHNKKENALLEFVRVVKAKEQV | 39 |
| VAGTMYYLTLEAKDGGKKKLYEAKVWVKWRAVRWWWGNFKELQEF | |
| KPVGDA | |
| (M)ATGVRAVPGNENSLEIEELARFAVDEHNKKENALLEFVRVVKAKEQV | 40 |
| VAGTMYYLTLEAKDGGKKKLYEAKVWVKVVGKWWVNINFKELQEFKP | |
| VGDA | |
| (M)ATGVRAVPGNENSLEIEELARFAVDEHNKKENALLEFVRVVKAKEQV | 41 |
| VAGTMYYLTLEAKDGGKKKLYEAKVWVKARYVVYYRWNFKELQEFKP | |
| VGDA | |
| (M)ATGVRAVPGNENSLEIEELARFAVDEHNKKENALLEFVRVVKAKEQV | 42 |
| VAGTMYYLTLEAKDGGKKKLYEAKVWVKGRYSVYWYVNFKELQEFKP | |
| VGDA | |
| (M)ATGVRAVPGNENSLEIEELARFAVDEHNKKENALLEFVRVVKAKEQV | 43 |
| VAGTMYYLTLEAKDGGKKKLYEAKVWVKVRYPPNIWKNFKELQEFKP | |
| VGDA | |
| (M)ATGVRAVPGNENSLEIEELARFAVDEHNKKENALLEFVRMVKAKEQ | 44 |
| YISVDKWVATMYYLTLEAKDGGKKKLYEAKVWVKYGGSCWRGGNFKELQ | |
| EFKPVGDA | |
| (M)ATGVRAVPGNENSLEIEELARFAVDEHNKKENALLEFVRVVKAKEQV | 45 |
| VAGTMYYLTLEAKDGGKKKLYEAKVWVKVAAYPRASINFKELQEFKPV | |
| GDA | |
| (M)ATGVRAVPGNENSLEIEELARFAVDEHNKKENALLEFVRVVKAKEQV | 46 |
| VAGTMYYLTLEAKDGGKKKLYEAKVWVKIGIPIRIWSNFKELQEFKPVG | |
| DA | |
| (M)ATGVRAVPGNENSLEIEELARFAVDEHNKKENALLEFVRVVKAKEQV | 47 |
| VAGTMYYLTLEAKDGGKKKLYEAKVWVKVNIYVISPRNFKELQEFKPV | |
| GDA | |
| (M)ATGVRAVPGNENSLEIEELARFAVDEHNKKENALLEFVCVVKAKEQV | 48 |
| VAGTMYYLTLEAKDGGKKKLYEAKVWVKVAPIYSVDSNFKELQEFKPV | |
| GDA | |
| (M)ATGVRAVPGNENSLEIEELARFAVDEHNKKENALLEFVRVVKAKEQV | 49 |
| VAGTMYYLTLEAKDGGKKKLYEAKVWVKSVAIRNWYWNFKELQEFKP | |
| VGDA | |
| (M)ATGVRAVPGNENSLEIEELARFAVDEHNKKENALLEFVRVVKAKEQV | 50 |
| VAGTMYYLTLEAKDGGKKKLYEAKVWVKVRRYSPPNRNFKELQEFKPV | |
| GDA | |
| (M)ATGVRAVPGNENSLEIEELARFAVDEHNKKENALLEFVRVVKAKEQV | 51 |
| VAGTMYYLTLEAKDGGKKKLYEAKVWVKVRYWVDRRKNFKELQEFKP | |
| VGDA | |
| (M)ATGVRAMPGNENSLEIEELARFAVDEHNKKENALLEFVRVVKAKEQV | 52 |
| VAGTMYYLTLEAKDGGKKKLYEAKVWVKVWWVVSDPANFKELQEFKP | |
| VGDA | |
| (M)ATGVRAVPGNENSLEIEELARFAVDEHNKKENALLEFVRVVKAKEQR | 53 |
| ARYYYVNRTMYYLTLEAKDGGKKKLYEAKVWVKWVIRGDSPANFKEL | |
| QEFKLVGDA | |

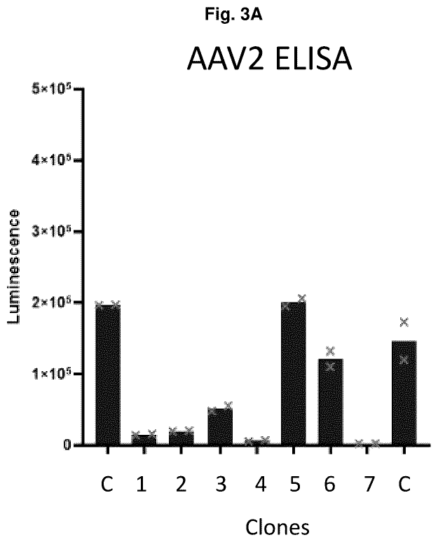

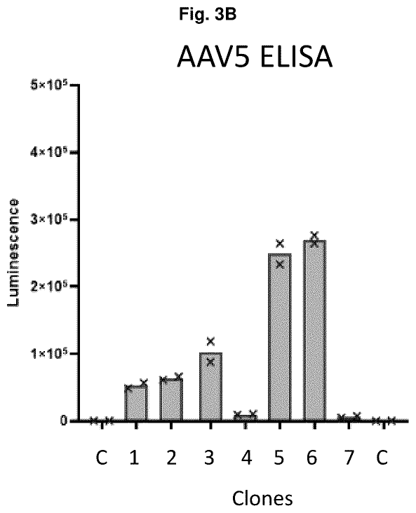

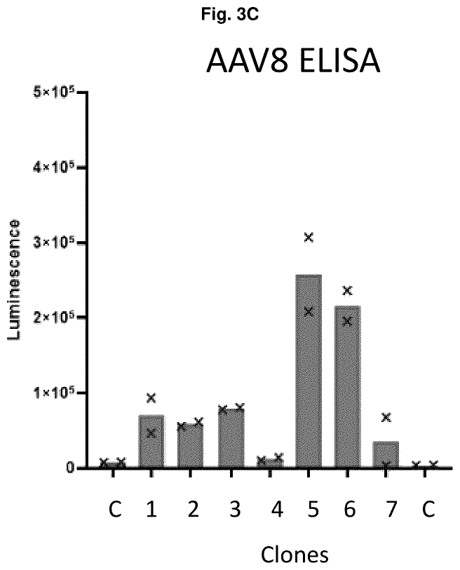

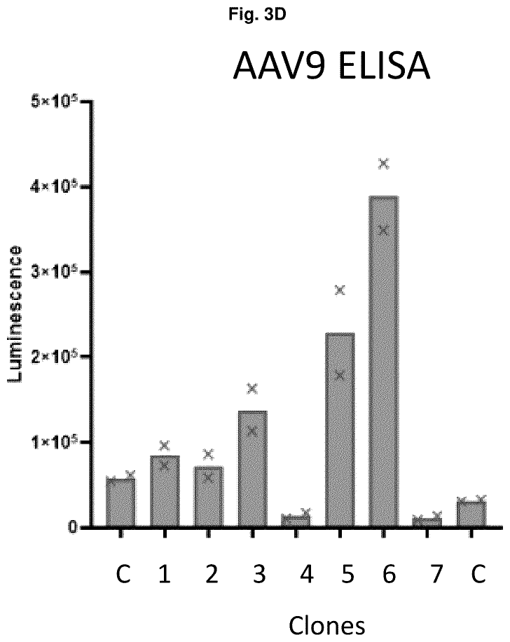

Seven of the variants of Table C were screened for cross-reactivity to AAV2, AAV5, and AAV8. Five variants showed strong cross-reactivity (FIGS. 3A-D). Two of the variants showed strong cross-reactivity to AAV2 in addition to AAV5, AAV8, and AAV9 capsids. In contrast, the control sequence known to bind AAV9, only cross reacted strongly to AAV2. The control sequence (labeled ‘C’) bound poorly or very weakly to AAV5 or AAV8. This data represents the potential of the variants for binding to multiple AAV serotypes.

The pH dependence of binding of the polypeptides of Table C was evaluated by ELISA. Initially, the bacterial lysates of cultures expressing the polypeptides of Table C were incubated in a citric acid solution (pH 4.0) containing AAV9 capsids for 30 minutes. Subsequently, the solution was washed in Tris-Buffered Saline (TBS), which contained 0.05% TWEEN® 20, at neutral pH. Five polypeptides, which showed reduce AAV9 binding signal, were selected for further characterization.

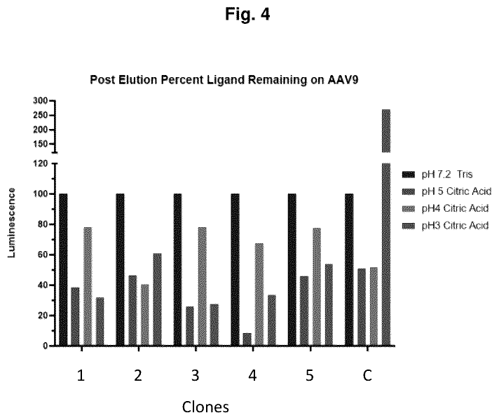

The five polypeptides were expressed and purified via a C-terminal 6X-His tag. The purified polypeptides (1 μM) were incubated with an ELISA plate that was coated with AAV9 full capsids. The polypeptides were eluted from the ELISA plate with a citric acid solution at pH 3, 4, or 5 (acidic elution) or in the TBS solution described above at pH 7.2 (neutral elution). The ELISA signal from the protein that was still bound to the AAV9 capsid was measured. The post elution % ligand remaining bound to AAV9 was calculated by dividing the signal after pH 3, 4, or 5 incubation by the signal after pH 7.2 incubation, and multiplying by 100. All five variants and the PKD2 control protein assayed showed reduced AAV9 binding signal after the acidic elution step compared to a neutral elution step (FIG. 4). This indicates that polypeptide binding to AAV9 capsids is pH sensitive.

### Example 2. Use of Polypeptides of Table C for Preferential Binding to Full Capsids

Purpose: The polypeptides of Table C were screened for polypeptides, which showed specific binding to full AAV capsids.

Methods: AAV capsids or human serum albumin (HSA) was coated to 96-well plates by overnight incubation at 4° C. degrees and then blocked. E. coli expression cultures expressing the His-tagged scaffolds were lysed and applied to the coated plates for one hour at room temperature. The plates were washed, and 100 μL of an anti-His tag antibody, which was conjugated to horseradish peroxide was added to the plates for one hour. The plates were washed, and substrate was added to develop the luminescence signal. Binding of the AAV capsid to scaffold was measured by reading luminescence intensity (Relative Luminescence Unit or RLU) on a Biotek Synergy II plate reader. Each of the 44 polypeptides of Table C bound to full AAV capsids.

These polypeptides were expressed in bacteria and induced with isopropyl β-D-1-thiogalactopyranoside to express protein IPTG and purified. The cell pellets from the bacteria were lysed, and the lysate was incubated with either AAV full capsids or AAV empty capsids. For evaluation of these variants, we used a calculation termed AAV FCBI (Full Capsids Bias Index). FCBI was calculated by the following equation: FCBI=((AAV Full Capsids RLU of Sample)/(AAV Full Capsids RLU of Control))/((AAV Empty Capsids RLU of Sample)/(AAV Empty Capsids RLU of Control)). The control is an N-terminal His-tagged polycystic kidney domain 2 (PKD2 domain) from the AAV receptor. The PKD2 domain is known to bind AAV with no discrimination between full and empty capsids.

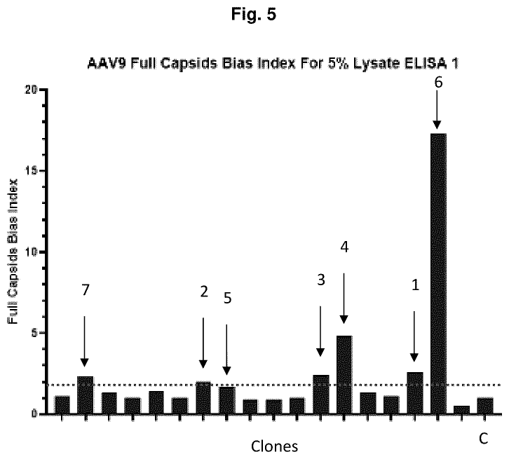

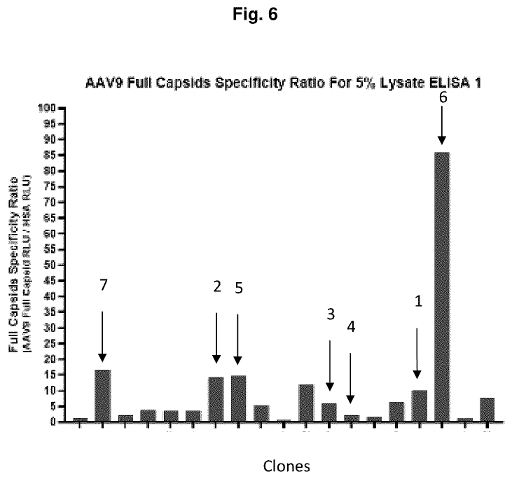

Results: Seven polypeptides (marked with arrows in FIG. 5 and FIG. 6) bound to the AAV full capsids with at least two-fold greater binding affinity than to empty capsids. (FIG. 5). An AAV Specificity Ratio (AAV SR) was also plotted (FIG. 6) and calculated by the following equation: AAV SR=(AAV full capsids RLU)/(human serum albumin RLU).

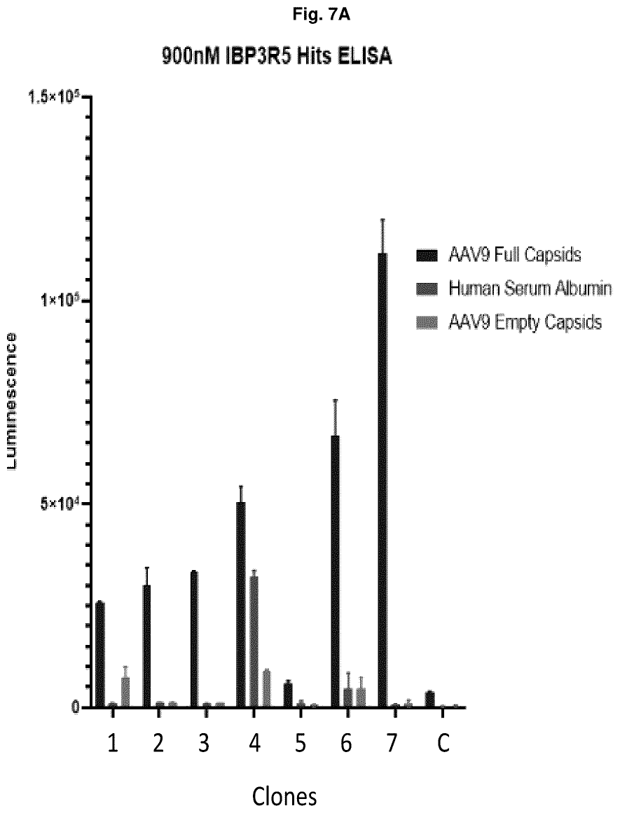

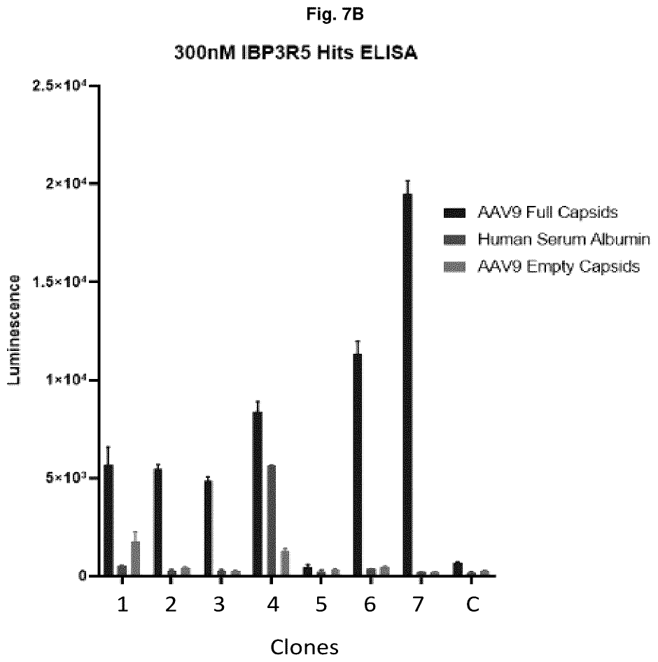

The polypeptides were expressed in E. Coli and purified via a 6X His-tag. The binding signal of the polypeptides to AAV full capsids, AAV empty capsids, and human serum albumin was evaluated at polypeptide concentrations of 900 nM (FIG. 7A) and 300 nM (FIG. 7B). At a polypeptide concentration of 900 nM, the AAV FCBI of all seven polypeptides was higher than that of the non-specific AAV control ligand. At a polypeptide concentration of 300 nM, the AAV FCBI of five polypeptides was higher than that of the non-specific AAV control ligand.

### Numbered Embodiments of the Disclosure

Notwithstanding the appended claims, the disclosure sets forth the following numbered embodiments:

1. A polypeptide comprising a sequence CX₁X₂X₃X₄FX₃CX₆X₇X₈X₉X₁₀CX₁₁X₁₂X₁₃ TWVCDGX₁₄X₁₃DCX₁₆DX₁₇X₁₈DEX₁₉X₂₀CTPTP, wherein each of X₁-X₂₉ are independently an amino acid, provided that at least 1, 2, 3, 4, or 5 amino acids of X₁-X₂₉ do not correspond to the amino acid of a wild-type avimer sequence.

2. The polypeptide of embodiment 1, wherein each of X₁-X₂₉ are independently D, I, P, V, W, S, A, K, R, N, or G.

3. The polypeptide of embodiment 1 or 2, wherein each of X₁-X₄ are independently D, I, P, W, V, K, I, A, or N.

4. The polypeptide of any one of embodiments 1-3, wherein:
X₁ is D, W, or A;
X₂ is I or V;
X₃ is P, K, or D; and
X₄ is P, I, or N.

5. The polypeptide of any one of embodiments 1-4, wherein X₅ is V, R, or P.

6. The polypeptide of embodiments 1-5, wherein each of X₆-X₁₀ are independently W, P, S, N, I, V, G, W, Y, or D.

7. The polypeptide of any one of embodiments 1-6, wherein:
X₆ is W or N;
X₇ is W or I;
X₈ is P, V, or Y;
X₉ is S, G, or Y;
X₁₀ is W, V, or D.

8. The polypeptide of any one of embodiments 1-7, wherein each of X₁₁, X₁₂, and X₁₃ are independently V, I, W, A, or G.

9. The polypeptide of any one of embodiments 1-8, wherein:
X₁₁ is V or I;
X₁₂ is W, V, or G;
X₁₃ is A or N.

The polypeptide of any one of embodiments 1-9, wherein each of X₁₄ and X₁₅ are independently A, P, R, V, or I.

11. The polypeptide of any one of embodiments 1-10, wherein:
X₁₄ is A, R, or I; and
X₁₅ is P, V, or I.

12. The polypeptide of any one of embodiments 1-11, wherein X₁₆ is I or S.

13. The polypeptide of any one of embodiments 1-12, wherein each of X₁₇ and X₁₈ are independently N, Y, P, I, or A.

14. The polypeptide of any one of embodiments 1-13, wherein:
X₁₇ is N, P, or I; and
X₁₈ is Y, P, or A.

15. The polypeptide of any one of embodiments 1-14, wherein each of X₁₉ and X₂₀ are independently W, I, G, Y or D.

16. The polypeptide of any one of embodiments 1-15, wherein:
X₁₉ is W, G, or Y; and
X₂₀ is I, W, or D.

17. The polypeptide of any of embodiments 1-10, comprising a sequence:
CDIPPFVCWWPSWCVWATWVCDGAPDCIDNYDEWICTPTP;
CWVKIFRCNIVGVCIVNTWVCDGRVDCSDPPDEGWCTPTP; or
CAIDNFPCNWYIDCIGATWVCDGIIDCIDIADEYDCTPTP; OR
   a sequence that is 90%, 95%, 96%, 97%, 98%, or 99% identity thereto.

18. A polypeptide comprising a sequence

ATGVRAVPGNENSLEIEELARFAVDEHNKKENALLE
FVRVVKAKEQVVAGTMYYLTLEAKDGGKKKLYEAKV
WVKU₁U₂U₃U₄U₅U₆U₇U₈U₉NFKELQEFKPVGDA,

wherein each of U₁-U₉ are independently an amino acid, provided that at least 1, 2, 3, 4, or 5 amino acids of U₁-U₉ do not correspond to the amino acid of a wild-type affimer sequence.

19. The polypeptide of embodiment 18, wherein each of U₁-U₉ are independently I, G, A, S, V, W, R, Y, D, K, P, V, K or C.

20. The polypeptide of embodiment 18 or 19, wherein U₁ is I, G, A, S, V, W, or A.

21. The polypeptide of any one of embodiment 18-20, wherein U₂ is R, V, G, A, S, I, P, Y, or N.

22. The polypeptide of any one of embodiment 18-21, wherein U₃ is A, I, Y, G, R, S, V, D, P, or K.

23. The polypeptide of any one of embodiment 18-22, wherein U₄ is I, Y, P, V, D, R, K, S, P, or W.

24. The polypeptide of any one of embodiment 18-23, wherein U₅ is V, A, G, Y, R, I, W, P, C or S.

The polypeptide of any one of embodiment 18-24, wherein U₆ is Y, R, V, W, I, V, S, N, P, or D.

26. The polypeptide of any one of embodiment 18-25, wherein U₇ is W, S, Y, V, R, I, A, or P.

27. The polypeptide of any one of embodiment 18-26, wherein U₈ is S, Y, K, A, P, W, R, N, or D.

28. The polypeptide of any one of embodiment 18-27, wherein U₉ is G, S, A, P, W, R, Y, I, V, or K.

29. The polypeptide of any of embodiments 18-28, comprising a sequence:

ATGVRAVPGNENSLEIEELARFAVDEHNKKENALLEFVRVVKAKEQVVA
GTMYYLTLEAKDGGKKKLYEAKVWVKIRAIVYWSGNFKELQEFKPVGDA;
ATGVRAVPGNENSLEIEELARFAVDEHNKKENALLEFVRVMKAKEQVVA
GTMYYLTLEAKDGGKKKLYEAKVWVKGVIYAYSYSNFKELQEFKPVGDA;
ATGVRAVPGNENSLEIEELARFAVDEHNKKENALLEFVRVVKAKEQVVA
GTMYYLTLEAKDGGKKKLYEAKVWVKAGYIGRSKANFKELQEFKPVGDA;
ATGVRAVPGNENSLEIEELARFAVDEHNKKENALLEFVRVVKAKEQVVA
GTMYYLTLEAKDGGKKKLYEAKVWVKAAGYYVYAPNFKELQEFKPVGDA;
ATGVRAVPGNENSLEIEELARFAVDEHNKKENALLEFVRVVKAKEQVVA
GTMYYLTLEAKDGGKKKLYEAKVWVKISRPGWWPWNFKELQEFKPVGDA;
ATGVRAVPGNENSLEIEELARFAVDEHNKKENALLEFVRVVKAKEQVVA
GTMYYLTLEAKDGGKKKLYEAKVWVKSRSVRIWYSNFKELQEFKPVGDA;
ATGVRAVPGNENSLEIEELARFAVDEHNKKENALLEFVRVVKAKEQVVA
GTMYYLTLEAKDGGKKKLYEAKVWVKVRRDIVVPRNFKELQEFKPVGDA;
ATGVRAVPGNENSLEIEELARFAVDEHNKKENALLEFVRVVKAKEQVVA
GTMYYLTLEAKDGGKKKLYEAKVWVKVRDIVSIRGNFKELQEFKPVGDA;
ATGVRAVPSNENSLEIEELARFAVDEHNKKENALLEFVRVVKAKEQVVA
GTMYYLTLEAKDGGKKKLYEAKVWVKAIKYVRYKANFKELQEFKPVGDA;
ATGVRAVPGNENSLEIEELARFAVDEHNKKENALLEFVRVVKAKEQVVA
GTMYYLTLEAKDGGKKKLYEAKVWVKSASYYVYSYNFKELQEFKPVGDA;
ATGVRAVPGNENSLEIEELARFAVDEHNKKENALLEFVRVVKAKEQVVA
GTMYYLTLEAKDGGKKKLYEAKVWVKWRAVRWWWGNFKELQEFKPVGDA;
ATGVRAVPGNENSLEIEELARFAVDEHNKKENALLEFVRVVKAKEQVVA
GTMYYLTLEAKDGGKKKLYEAKVWVKVVGKWWVNINFKELQEFKPVGDA;
ATGVRAVPGNENSLEIEELARFAVDEHNKKENALLEFVRVVKAKEQVVA
GTMYYLTLEAKDGGKKKLYEAKVWVKARYVVYYRWNFKELQEFKPVGDA;
ATGVRAVPGNENSLEIEELARFAVDEHNKKENALLEFVRVVKAKEQVVA
GTMYYLTLEAKDGGKKKLYEAKVWVKGRYSVYWYVNFKELQEFKPVGDA;
ATGVRAVPGNENSLEIEELARFAVDEHNKKENALLEFVRVVKAKEQVVA
GTMYYLTLEAKDGGKKKLYEAKVWVKVRYPPNIWKNFKELQEFKPVGDA;
ATGVRAVPGNENSLEIEELARFAVDEHNKKENALLEFVRVVKAKEQVVA
GTMYYLTLEAKDGGKKKLYEAKVWVKVAAYPRASINFKELQEFKPVGDA;
ATGVRAVPGNENSLEIEELARFAVDEHNKKENALLEFVRVVKAKEQVVA
GTMYYLTLEAKDGGKKKLYEAKVWVKIGIPIRIWSNFKELQEFKPVGDA;
ATGVRAVPGNENSLEIEELARFAVDEHNKKENALLEFVRVVKAKEQVVA
GTMYYLTLEAKDGGKKKLYEAKVWVKVNIYVISPRNFKELQEFKPVGDA;
ATGVRAVPGNENSLEIEELARFAVDEHNKKENALLEFVCVVKAKEQVVA
GTMYYLTLEAKDGGKKKLYEAKVWVKVAPIYSVDSNFKELQEFKPVGDA;
ATGVRAVPGNENSLEIEELARFAVDEHNKKENALLEFVRVVKAKEQVVA
GTMYYLTLEAKDGGKKKLYEAKVWVKSVAIRNWYWNFKELQEFKPVGDA;
ATGVRAVPGNENSLEIEELARFAVDEHNKKENALLEFVRVVKAKEQVVA
GTMYYLTLEAKDGGKKKLYEAKVWVKVRRYSPPNRNFKELQEFKPVGDA;
or
AATGVRAVPGNENSLEIEELARFAVDEHNKKENALLEFVRVVKAKEQVV
AGTMYYLTLEAKDGGKKKLYEAKVWVKVRYWVDRRKNFKELQEFKPVGD
A;

or
a sequence that is 90%, 95%, 96%, 97%, 98%, or 99% identity thereto.

30. A polypeptide comprising a sequence ATGVRAVPGNENSLEIEELARFAVDEHNKKENALLEFVRVVKAKEQZ₁Z₂Z₃Z₄Z₅Z₆Z₇Z₈Z₉TMYYLTLEAKDGGKKKLYEAKVWVKU₁U₁₂U₃U₄U₅U₆U₇U₈U₉NFKELQEFKPVG DA, wherein each of Z₁-Z₉ and each of U₁-U₉ are independently an amino acid, provided that at least 1, 2, 3, 4, or 5 amino acids of Z₁-Z₉ and U₁-U₉ do not correspond to the amino acid of a wild-type affimer sequence.

31. The polypeptide of any one of embodiment 30, wherein each of U₁-U₉ is independently an amino acid of embodiments 19-28.

32. The polypeptide of any one of embodiment 30, wherein each of U₁-U₉ is independently Y, G, S, C, W, R, V, I, D, P, or A.

33. The polypeptide of any one of embodiment 32, wherein
U₁ is Y or W;
U₂ is G or V;
U₃ is G or I;
U₄ is S or R;
U₅ is C or G;
U₆ is W or D;
U₇ is R or S;
U₈ is G or P; or
U₉ is G or A.

34. The polypeptide of any one of embodiments 30-33, wherein each of Z₁-Z₉ is independently Y, I, S, V, D, K, W, A, R, or N.

35. The polypeptide of any one of embodiments 30-34, wherein Z₁ is Y or R.

36. The polypeptide of any one of embodiments 30-35, wherein Z₂ is I or A.

37. The polypeptide of any one of embodiments 30-36, wherein Z₃ is S or R.

38. The polypeptide of any one of embodiments 30-37, wherein Z₄ is V or Y.

39. The polypeptide of any one of embodiments 30-38, wherein Z₅ is D or Y.

40. The polypeptide of any one of embodiments 30-39, wherein Z₆ is K or Y.

41. The polypeptide of any one of embodiments 30-40, wherein Z₇ is W or V.

42. The polypeptide of any one of embodiments 30-41, wherein Z₈ is V or N.

43. The polypeptide of any one of embodiments 30-42, wherein Z₉ is A or R.

44. The polypeptide of any one of embodiments 30-43, comprising a sequence:
ATGVRAVPGNENSLEIEELARFAVDEHNKKENALLEFVRMVKAKEQYISVD KWVATMYYLTLEAKDGGKKKLYEAKVWVKYGGSCWRGGNFKELQEFKP VGDA; or
ATGVRAVPGNENSLEIEELARFAVDEHNKKENALLEFVRVVKAKEQRARYY YVNRTMYYLTLEAKDGGKKKLYEAKVWVKWVIRGDSPANFKELQEFKLV GDA; or
a sequence that is 90%, 95%, 96%, 97%, 98%, or 99% identity thereto.

A polypeptide comprising a sequence

ATGVRAB₁B₂GNENB₃LEIEELARFAVDEHNKKENALLEFVRVVK
AKEQVVAB₄TMYYLTLEAKDGGKKKLYEAKVWVKU₁U₂U₃U₄U₅U₆
U₇U₈U₉NFKELQEFKPVGDA,

wherein each of B₁, B₂, B₃, B₄, and U₁-U₉ are independently an amino acid, provided that at least 1, 2, 3, 4, or 5 amino acids of B₁-B₄ and U₁-U₉ do not correspond to the amino acid of a wild-type affimer sequence.

46. The polypeptide of embodiment 45, wherein B₁ is M or V.

47. The polypeptide of embodiment 45 or 46, wherein B₁ is M.

48. The polypeptide of anyone of embodiments 45-47, wherein B₂ is S or P.

49. The polypeptide of anyone of embodiments 45-48, wherein B₂ is S.

50. The polypeptide of anyone of embodiments 45-49, wherein B₃ is G or S.

51. The polypeptide of anyone of embodiments 45-50, wherein B₃ is G.

52. The polypeptide of anyone of embodiments 45-51, wherein B₄ is C or G.

53. The polypeptide of anyone of embodiments 45-52, wherein B₄ is C.

54. The polypeptide of any one of embodiments 45-53, wherein each of U₁-U₉ is independently an amino acid of embodiments 19-28.

55. The polypeptide of any one of embodiments 42-54, wherein each of U₁-U₉ is independently R, I, V, A, W, S, P, Y, K, or G.

56. The polypeptide of any one of embodiments 42-55, wherein
U₁ is R, S, or V;
U₂ is I, R, P, or W;
U₃ is V or W;
U₄ is R, Y, or V;
U₅ is V or W;
U₆ is A, I, P, or S;
U₇ is V, R, K, or D;
U₈ is W, G, or P; or
U₉ is V, G, or A.

57. The polypeptide of any one of embodiments 42-56, comprising a sequence selected from:

ATGVRAVSGNENGLEIEELARFAVDEHNKKENALLEFVRVVKAKEQVVA
GTMYYLTLEAKDGGKKKLYEAKVWVKRIVRVAVWVNFRELQEFKPVGDA;
ATGVRAVPGNENSLEIEELARFAVDEHNKKENALLEFVRVVKAKEQVVA
CTMYYLTLEAKDDGKKKLYEAKVWVKSRVRVIRWGNFKELQEFKPVGDA;
ATGVRAVSGNENSLEIEELARFAVDEHNKKENALLEFVRVVKAKEQVVA
GTMYYLTLEAKDGGKKKLYEAKVWVKSPVYWPKGGNFKELQEFKPVGDA;
or
ATGVRAMPGNENSLEIEELARFAVDEHNKKENALLEFVRVVKAKEQVVA
GTMYYLTLEAKDGGKKKLYEAKVWVKVWWVVSDPANFKELQEFKPVGDA;

a sequence that is 90%, 95%, 96%, 97%, 98%, or 99% identity thereto.

58. A polypeptide having a sequence ATGVRAB₁B₂GNENB₃LEIEELARFAVDEHNKKENALLEFVRVVKAKEQZ₁Z₂Z₃Z₄Z₅Z₆Z₇Z₈Z₉TMYYLTLEAKDGGKKKLYEAKVWVKU₁U₂U₃U₄U₅U₆U₇U₈U₉NFKELQEFKP VGDA, wherein each of B₁, B₂, B₃, Z₁-Z₉ and U₁-U₉ are independently an amino acid, provided that at least 1, 2, 3, 4, or 5 amino acids of B₁-B₄, Z₁-Z₉ and U₁-U₉ do not correspond to the amino acid of a wild-type affimer sequence.

59. The polypeptide of embodiment 58, wherein each of B₁-B₃ are independently an amino acid of one of embodiments 46-53.

60. The polypeptide of embodiment 58 or 59, wherein each of Z₁-Z₉ are independently an amino acid of one of embodiments 34-43.

61. The polypeptide of any one of embodiments 58-60, wherein each of U₁-U₉ are independently an amino acid of embodiments 19-28, 32, 33, 55, or 56.

62. A polypeptide comprising a sequence VDNKENKEJ₁J₂J₃AJ₄J₅EIJ₆J₇LPNLNJ₈J₉QJ₁₀J₁₁AFIJ₁₂SLJ₁₃DDPJ₁₄J₁₅J₁₆ANLLAEAK KLNDAQAPK, wherein each of J₁-J₁₆ is independently an amino acid, provided that at least 1, 2, 3, 4, or 5 amino acids of J₁-J₁₆ do not correspond to the amino acid of a wild-type affibody sequence.

63. The polypeptide of embodiment 62, wherein each of J₁-J₁₆ are independently Q, W, L, Y, H, A, M, R, E, K, V, S, Q, R, or P.

64. The polypeptide of embodiment 62 or 63, wherein each of J₁-J₃ are independently Q, W, L, Y, A, S, V, E, M, K, V, H, or R.

55. The polypeptide of any one of embodiments 62-64, wherein J₁ is Q, W, Y, E, L, M, or S.

66. The polypeptide of any one of embodiments 62-65, wherein J₂ is W, Y, S, L, A, M, Q, V, or R.

67. The polypeptide of any one of embodiments 62-66, wherein J₃ L, A, V, M, Y, W, or H.

68. The polypeptide of any one of embodiments 62-67, wherein each of J₄ and J₅ is independently W, E, M, H, V, A, Y, L, or S.

69. The polypeptide of any one of embodiments 62-68, wherein J₄ is W, E, M, H, V, A, or Y.

70. The polypeptide of any one of embodiments 62-69, wherein J₅ is Y, L, M, V, S, or A.

71. The polypeptide of any one of embodiments 62-70, wherein each of J₆ or J₇ is H, W, Y, E, L, Q, A, M, R, or S.

72. The polypeptide of any one of embodiments 62-71, wherein J₆ is H, W, Y, E, L, Q, A, Y, M, R, or S.

73. The polypeptide of any one of embodiments 62-72, wherein J₇ is Y, W, L, R, S, or A.

74. The polypeptide of any one of embodiments 62-73, wherein each of J₈ and J₉ are independently A, M, S, L, H, K, Y, Q, or W.

75. The polypeptide of any one of embodiments 62-74, wherein J₈ is A, M, S, L, or H.

76. The polypeptide of any one of embodiments 62-74, wherein J₉ is M, K, Y, Q, W, or L.

77. The polypeptide of any one of embodiments 62-76, wherein each of J₁₀ and J₁₁ are independently H, W, L, M, E, S, A, R, Y, or V.

78. The polypeptide of any one of embodiments 62-77, wherein J₁₀ is H, W, L, M, E, S or A.

79. The polypeptide of any one of embodiments 62-78, wherein J₁₁ is R, Y, V, A, L, or E.

80. The polypeptide of any one of embodiments 62-79, wherein J₁₂ is L, Y, V, E, M, or Q.

81. The polypeptide of any one of embodiments 62-80, wherein J₁₃ is Y, E, V, R, W, Q, or H.

82. The polypeptide of any of embodiments 62-81, wherein each of J₁₄-J₁₆ are independently R, P, H, S or Q.

83. The polypeptide of any of embodiments 62-82, wherein J₁₄ is R or S.

84. The polypeptide of any of embodiments 62-83, wherein J₁₅ is R or Q.

85. The polypeptide of any of embodiments 62-84, wherein J₁₆ is H or S or P or R.

86. The polypeptide of any one of embodiments 62-85, comprising a sequence selected from:
VDNKFNKEQWLAWYEIHYLPNLNAMQHRAFILSPYDDPSQSANLLAEAKKL NDAQAPK;
VDNKFNKEWYAAELEIWELPNLNMKQWYAFIYSLEDDPSQSANLLAEAKKL NDAQAPK;
VDNKFNKEYSVAWLEIYLLPNLNMMQLRAFIVSLEDDPSQSANLLAEAKKLN DAQAPK;
VDNKFNKEELMAMYEIEYLPNLNAYQMVAFIVSLVDDPSQSANLLAEAKKL NDAQAPK
VDNKFNKELAYAHMEIWYLPNLNSQQWYAFIESLRDDPSQSANLLAEAKKL NDAQAPK
VDNKFNKEMSYAVLEILRLPNLNLMQLAFIYSLWDDPSQSANLLAEAKKLND AQAPK
VDNKFNKEYYYAAVEIQYLPNLNMWQMLAFIVSLYDDPRRPANLLAEAKKL NDAQAPK
VDNKFNKEMYVAVLEIHSLPNLNLMQLVAFILSLYDDPSQSANLLAEAKKLN DAQAPK
VDNKFNKEWSWAYSEIHLLPNLNHLQHYAFIMSLQDDPRRHANLLAEAKKL NDAQAPK
VDNKFNKEYMLAELEIAALPNLNHWQEVAFIVSLVDDPSQSANLLAEAKKLN DAQAPK
VDNKFNKESQYAYAEIYLLPNLNHWQSVAFIWSLYDDPSQSANLLAEAKKL NDAQAPK
VDNKFNKEKVHAMLEIRWLPNLNMLQAYAFIOSLHDDPSQSANLLAEAKK LNDAQAPK
VDNKFNKEERAAYVEIHWLPNLNSKQWEAFIMSLLODDPSQSANLLAEAKKL NDAQAPK; or
a sequence with at least 80%, at least 85%, at least 90%, at least 91%, at least 92%, at least 93%, at least 94%, at least 95%, at least 96%, at least 97%, at least 98%, at least 99%, or 100% identity to any one of the aforementioned sequences.

87. A method of binding an AAV capsid, comprising contacting AAV capsids with a polypeptide of anyone of embodiments 1-86.

88. The method of embodiment 87, wherein the AAV is AAV9.

89. The method of embodiment 87 or 88, wherein the AAV capsid is full.

90. A method of separating full AAV capsids from empty AAV capsids, the method comprising:
(a) contacting a polypeptide with a solution comprising the mixture of full AAV capsids and empty AAV capsids, wherein the polypeptide shows at least a two-fold increase in binding preference for full AAV capsids versus empty AAV capsids;
(b) allowing the polypeptide to bind the full AAV capsids;
(c) separating empty AAV capsids from the full AAV capsids bound to the polypeptide.

91. The method of embodiment 90, wherein the polypeptide comprises an avimer, affimer, or affibody.

92. The method of embodiment 90 or 91, wherein the polypeptide is a polypeptide of any one of embodiments 1-86.

93. The method of any one of embodiments 90-92, further comprising quantifying full AAV capsids in the sample.

94. The method of any one of embodiments 90-93, further comprising determining a ratio of full AAV capsids to empty AAV capsids in the sample.

95. The method of any one of embodiments 90-94, further comprising quantifying the full virus particles in the sample.

96. The method of any one of embodiments 90-95, wherein the solution is step (a) comprises a cell lysate.

97. The method of any one of embodiments 90-96, wherein the solution in step (a) is clarified.

98. A polypeptide comprising a sequence CX₁X₂X₃X₄FX₃CX₆X₇X₈X₉X₁₀CX₁₁X₁₂X₁₃ TWVCDGX₁₄X₁₃DCX₁₆DX₁₇X₁₈DEX₁₉X₂₀CTPTP (SEQ ID NO: 54), wherein each of X₁-X₂₀ are independently an amino acid, provided that at least 1, 2, 3, 4, or 5 amino acids of X₁-X₂₀ do not correspond to the amino acid of a wild-type avimer sequence.

99. The polypeptide of claim 98, wherein each of X₁-X₂₀ are independently D, I, P, V, W, S, A, K, R, N, Y, or G.

100. The polypeptide of claim 98 or 99, wherein:
X₁ is D, W, or A;
X₂ is I or V;
X₃ is P, K, or D;
X₄ is P, I, or N;
X₅ is V, R, or P;
X₆ is W or N;
X₇ is W or I;
X₈ is P, V, or Y;
X₉ is S, G, or I;
X₁₀ is W, V, or D;
X₁₁ is V or I;
X₁₂ is W, V, or G;
X₁₃ is A or N;
X₁₄ is A, R, or I;
X₁₅ is P, V, or I;
X₁₆ is I or S;
X₁₇ is N, P, or I;
X₁₈ is Y, P, or A;
X₁₉ is W, G, or Y; and
X₂₉ is I, W, or D.

101. The polypeptide of any of claims 98-100, comprising the sequence of any one of SEQ ID NOS: 55-57, or a sequence that is at least 90%, at least 91%, at least 92%, at least 93%, at least 94%, at least 95%, at least 96%, at least 97%, at least 98%, or at least 99% identical to any one of SEQ ID NOS: 55-57.

102. A polypeptide comprising a sequence ATGVRAVPGNENSLEIEELARFAVDEHNKKENALLEFVRVVKAKEQVVAG TMYYLTLEAKDGGKKKLYEAKVWVKU₁U₂U₃U₄U₃U₆U₇U₈U₉NEKELQEFKPVGDA (SEQ ID NO: 58), wherein each of U₁-U₉ are independently an amino acid, provided that at least 1, 2, 3, 4, or 5 amino acids of U₁-U₉ do not correspond to the amino acid of a wild-type affimer sequence.

103. The polypeptide of claim 102, wherein each of U₁-U₉ are independently I, G, A, S, V, W, R, Y, D, K, P, V, or C.

104. The polypeptide of claim 102 or 103, wherein:
U₁ is I, G, A, S, V, W, or A;
U₂ is R, V, G, A, S, I, P, Y, or N;
U₃ is A, I, Y, G, R, S, V, D, P, or K;
U₄ is I, Y, P, V, D, R, K, S, P, or W;
U₅ is V, A, G, Y, R, I, W, P, C or S;
U₆ is Y, R, V, W, I, V, S, N, P, or D;
U₇ is W, S, Y, V, R, I, A, or P;
U₈ is S, Y, K, A, P, W, R, N, or D; and
U₉ is G, S, A, P, W, R, Y, I, V, or K.

105. The polypeptide of any of claims 102-104, comprising the sequence of any one of SEQ ID NOS: 59, 61-66, 68-76, and 78-80, or a sequence that is at least 90%, at least 91%, at least 92%, at least 93%, at least 94%, at least 95%, at least 96%, at least 97%, at least 98%, or at least 99% identical to any one of SEQ ID NOS: 59, 61-66, 68-76, and 78-80.

106. A polypeptide comprising a sequence ATGVRAVPGNENSLEIEELARFAVDEHNKKENALLEFVRB₉VKAKEQZ₁Z₂Z₃Z₄Z₅Z₆Z₇Z₈Z₉TMYYLTLEAKDGGKKKLYEAKVWVKU₁U₂U₃U₄U₅U₆U₇U₈U₉NFKELQEFKB₁₀V GDA, wherein each of Z₁-Z₉, each of B₉-B₁₀, and each of U₁-U₉ are independently an amino acid (SEQ ID NO: 105), provided that at least 1, 2, 3, 4, or 5 amino acids of Z₁-Z₉ and U₁-U₉ do not correspond to the amino acid of a wild-type affimer sequence.

107. The polypeptide of any one of claim 106, wherein each of U₁-U₉ is independently I, G, A, S, V, W, R, Y, D, K, P, V, or C.

108. The polypeptide of any one of claim 106, wherein each of U₁-U₉ is independently Y, G, S, C, W, R, V, I, D, P, or A.

109. The polypeptide of any one of claim 108, wherein
U₁ is Y or W;
U₂ is G or V;
U₃ is G or I;
U₄ is S or R;
U₅ is C or G;
U₆ is W or D;
U₇ is R or S;
U₈ is G or P; or
U₉ is G or A.

110. The polypeptide of any one of claims 106-109, wherein each of Z₁-Z₉ is independently Y, I, S, V, D, K, W, A, R, or N.

111. The polypeptide of any one of claims 106-110, wherein:
Z₁ is Y or R;
Z₂ is I or A;
Z₃ is S or R;
Z₄ is V or Y;
Z₅ is D or Y;
Z₆ is K or Y;
Z₇ is W or V;
Z₈ is V or N; and
Z₉ is A or R.

112. The polypeptide of any one of claims 106-111, wherein:
B₉ is V or M; and
B₁₀ is P or L.

113. The polypeptide of any one of claims 106-112, comprising the sequence of any one of SEQ ID NOS: 82-83 and 106, or a sequence that is at least 90%, at least 91%, at least 92%, at least 93%, at least 94%, at least 95%, at least 96%, at least 97%, at least 98%, or at least 99% identical to any one of SEQ ID NOS: 82-83 and 106.

114. A polypeptide comprising a sequence

ATGVRAB₁B₂B₆NENB₃LEIEELARFAVDEHNKKENALLEFVB₇VB₈KA
KEQVVAB₄TMYYLTLEAKDB₁₁GKKKLYEAKVWVKU₁U₂U₃U₄U₅U₆U₇U₈
U₉NFB₅ELQEFKPVGDA,

wherein each of B₁-B₈ and U₁-U₉ are independently an amino acid, provided that at least 1, 2, 3, 4, or 5 amino acids of B₁-134 and U₁-U₉ do not correspond to the amino acid of a wild-type affimer sequence.

115. The polypeptide of claim 114, wherein:
B₁ is M or V;
B₂ is S or P;
B₃ is G or S;
B₄ is C or G;
B₅ is R or K;
B₆ is G or S;
B₇ is R or C;
B₈ is V or M; and
B₁₁ is G or D.

116. The polypeptide of claim 114 or 115, wherein each of U₁-U₉ is independently I, G, A, S, V, W, R, Y, D, K, P, V, or C.

117. The polypeptide of any one of claims 114-116, wherein each of U₁-U₉ is independently R, I, V, A, W, S, P, Y, K, or G.

118. The polypeptide of any one of claims 114-117, wherein
U₁ is R, S, or V;
U₂ is I, R, P, or W;
U₃ is V or W;
U₄ is R, Y, or V;
U₅ is V or W;
U₆ is A, I, P, or S;
U₇ is V, R, K, or D;
U₈ is W, G, or P; or
U₉ is V, G, or A.

119. The polypeptide of any of claims 114-118, comprising the sequence of any one of SEQ ID NOS: 85-88, 60, 67, and 77, or a sequence that is at least 90%, at least 91%, at least 92%, at least 93%, at least 94%, at least 95%, at least 96%, at least 97%, at least 98%, or at least 99% identical to any one of SEQ ID NOS: 85-88, 60, 67, and 77.

120. A polypeptide having a sequence ATGVRAB₁B₂B₆NENB₃LEIEELARFAVDEHNKKENALLEFVB₇B₉B₈KAKEQZ₁Z₂Z₃Z₄Z₅Z₆Z₇Z₈Z₉TMYYLTLEAKDGGKKKLYEAKVWVKU₁U₂U₃U₄U₅U₆U₇U₈U₉NFB₅ELWEF KB₁₀VGDA, wherein each of B₁, B₂, B₃, B₅-B₁₀, Z₁-Z₉ and U₁-U₉ are independently an amino acid (SEQ ID NO: 107), provided that at least 1, 2, 3, 4, or 5 amino acids of B₁-B₃, B₅-B₁₀, and U₁-U₉ do not correspond to the amino acid of a wild-type affimer sequence.

121. The polypeptide of claim 120, wherein:
B₁ is M or V;
B₂ is S or P;
B₃ is G or S;
B₅ is K or R;
B₆ is G or S;
B₇ is R or C;
B₈ is V or M;
B₉ is V or M; and
B₁₀ is P or L.

122. The polypeptide of claim 120 or 121, wherein:
Z₁ is Y, S, or R;
Z₂ is I, A, or R;
Z₃ is Y, S, or R;
Z₄ is P, V, or Y;
Z₅ is K, D, or Y;
Z₆ is G, K, or Y;
Z₇ is N, W, or V;
Z₈ is K, V, or N; and
Z₉ is V, A, or R.

123. The polypeptide of any one of claims 120-122, wherein:
U₁ is I, G, A, S, V, W, or A;
U₂ is R, V, G, A, S, I, P, Y, or N;
U₃ is A, I, Y, G, R, S, V, D, P, or K;
U₄ is I, Y, P, V, D, R, K, S, P, or W;
U₅ is V, A, G, Y, R, I, W, P, C, or S;
U₆ is Y, R, V, W, I, V, S, N, P, or D;
U₇ is W, S, Y, V, R, I, A, or P;
U₈ is S, Y, K, A, P, W, R, N, or D; and
U₉ is G, S, A, P, W, R, Y, I, V, or K.

124. A polypeptide comprising a sequence VDNKFNKEJ₁J₂J₃AJ₄J₅EIJ₆J₇LPNLNJ₈J₉QJ₁₀J₁₁AFIJ₁₂SJ₁₇J₁₃DDPJ₁₄J₁₅J₁₆ANTLLAEAK KLNDAQAPK, wherein each of J₁-J₁₇ is independently an amino acid or absent (SEQ ID NO: 108), provided that at least 1, 2, 3, 4, or 5 amino acids of J₁-J₁₇ do not correspond to the amino acid of a wild-type affibody sequence.

125. The polypeptide of claim 124, wherein each of J₁-J₁₇ are independently Q, W, L, Y, H, A, M, R, E, K, V, S, Q, R, or P.

126. The polypeptide of claim 124 or 125, wherein:
J₁ is Q, W, Y, E, L, M, K, or S;
J₂ is W, Y, S, L, A, M, Q, V, or R;
J₃ is L, A, V, M, Y, W, or H;
J₄ is W, E, M, H, V, A, or Y;
J₅ is Y, L, M, V, S, or A;
J₆ is H, W, Y, E, L, Q, A, Y, M, R, or S;
J₇ is Y, W, L, R, S, E, or A;
J₈ is A, M, S, L, or H;
J₉ is M, K, Y, Q, W, or L;
J₁₀ is H, W, L, M, E, S, A, or absent;
J₁₁ is R, Y, V, A, L, or E;
J₁₂ is L, Y, V, W, E, M, or Q;
J₁₃ is Y, E, V, R, W, Q, or H;
J₁₄ is R or S;
J₁₅ is R or Q;
J₁₆ is H, S, P, or R; and
J₁₇ is L or P.

127. The polypeptide of any one of claims 124-126, comprising the sequence of any one of SEQ ID NOS: 91-103, or a sequence that is at least 90%, at least 91%, at least 92%, at least 93%, at least 94%, at least 95%, at least 96%, at least 97%, at least 98%, or at least 99% identical to any one of SEQ ID NOS: 91-103.

128. A polypeptide comprising an amino acid sequence with at least 90%, at least 91%, at least 92%, at least 93%, at least 94%, at least 95%, at least 96%, at least 97%, at least 98%, or at least 99% identity to any one of SEQ ID NOS: 55-57, 59-80, 82, 83, 85-88, 91-103, and 106.

129. A multimeric polypeptide comprising at least one polypeptide of any one of claims 98-128 and a polypeptide linker.

130. The multimeric polypeptide of claim 129, comprising from about 1 to about 10 polypeptides of any one of claims 98-128 and from about 1 to about 10 polypeptide linkers.

131. The multimeric polypeptide of claim 129 or claim 3130, comprising from N- to C-terminus: a first polypeptide linker, a first polypeptide of any one of claims 1-30.

132. The multimeric polypeptide of claim 33, comprising a second polypeptide linker that is C-terminal to the first polypeptide of any one of claims 98-128 and a second polypeptide of any one of claims 98-128, wherein the second polypeptide of any one of claims 98-128 is C-terminal to the second polypeptide linker of any one of claims 98-128.

133. The multimeric polypeptide of any one of claims 129-132, having an amino acid sequence with at least 90%, at least 91%, at least 92%, at least 93%, at least 94%, at least 95%, at least 96%, at least 97%, at least 98%, at least 99%, or 100% identity to any one of SEQ ID NOS: 169-193.

134. A nucleic acid encoding a polypeptide of any one of claims 98-128 or a multimeric polypeptide of any one of claims 129-133.

135. A method of binding an AAV capsid, comprising contacting an AAV capsid with a polypeptide of anyone of claims 98-128 or a multimeric polypeptide of any one of claims 129-133.

136. The method of claim 135, wherein the AAV is AAV9.

137. The method of claim 135 or 136, wherein the AAV capsid is full.

138. A method of separating full AAV capsids from empty AAV capsids, the method comprising:
(a) contacting a polypeptide with a solution comprising the mixture of full AAV capsids and empty AAV capsids, wherein the polypeptide shows at least a two-fold increase in binding preference for full AAV capsids versus empty AAV capsids;
(b) allowing the polypeptide to bind the full AAV capsids;
(c) separating empty AAV capsids from the full AAV capsids bound to the polypeptide.

139. The method of claim 138, wherein the polypeptide comprises an avimer, affimer, or affibody.

140. The method of claim 138 or 139, wherein the polypeptide is a polypeptide of any one of claims 98-128 or the multimeric polypeptide of any one of claims 129-133.

141. A nucleic acid with at least 90%, at least 91%, at least 92%, at least 93%, at least 94%, at least 95%, at least 96%, at least 97%, at least 98%, at least 99%, or 100% identity to any one of SEQ ID NOS: 194-195.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that it constitutes valid prior art or form part of the common general knowledge in any country in the world. The following references are incorporated by reference herein in their entireties: U.S. Pat. No. 11,015,174 and U.S. Publication No. 2019/0055523.

1. A polypeptide comprising a sequence CX₁X₂X₃X₄FX₅CX₆X₇X₈X₉X₁₀CX₁₁X₁₂X₁₃ TWVCDGX₁₄X₁₅DCX₁₆DX₁₇X₁₈DEX₁₉X₂₀CTPTP (SEQ ID NO: 54), wherein each of X₁-X₂₀ are independently an amino acid, provided that at least 1, 2, 3, 4, or 5 amino acids of X₁-X₂₀ do not correspond to the amino acid of a wild-type avimer sequence.

2. The polypeptide of claim 1, wherein each of X₁-X₂₀ are independently D, I, P, V, W, S, A, K, R, N, Y, or G.

3. The polypeptide of claim 1, wherein:
X₁ is D, W, or A;
X₂ is I or V;
X₃ is P, K, or D;
X₄ is P, I, or N;
X₅ is V, R, or P;
X₆ is W or N;
X₇ is W or I;
X₈ is P, V, or Y;
X₉ is S, G, or I;
X₁₀ is W, V, or D;
X₁₁ is V or I;
X₁₂ is W, V, or G;
X₁₃ is A or N;
X₁₄ is A, R, or I;
X₁₅ is P, V, or I;
X₁₆ is I or S;
X₁₇ is N, P, or I;
X₁₈ is Y, P, or A;
X₁₉ is W, G, or Y; and
X₂₀ is I, W, or D.

4. The polypeptide of claim 1, comprising the sequence of any one of SEQ ID NOS: 55-57, or a sequence that is at least 90%, at least 91%, at least 92%, at least 93%, at least 94%, at least 95%, at least 96%, at least 97%, at least 98%, or at least 99% identical to any one of SEQ ID NOS: 55-57.

5. A polypeptide comprising a sequence ATGVRAVPGNENSLEIEELARFAVDEHNKKENALLEFVRVVKAKEQVVAG TMYYLTLEAKDGGKKKLYEAKVWVKU₁U₂U₃U₄U₅U₆U₇U₈U₉NFKELQEFKPVGDA (SEQ ID NO: 58), wherein each of U₁-U₉ are independently an amino acid, provided that at least 1, 2, 3, 4, or 5 amino acids of U₁-U₉ do not correspond to the amino acid of a wild-type affimer sequence.

6. The polypeptide of claim 5, wherein each of U₁-U₉ are independently I, G, A, S, V, W, R, Y, D, K, P, V, or C.

7. The polypeptide of claim 5, wherein:
U₁ is I, G, A, S, V, W, or A;
U₂ is R, V, G, A, S, I, P, Y, or N;
U₃ is A, I, Y, G, R, S, V, D, P, or K;
U₄ is I, Y, P, V, D, R, K, S, P, or W;
U₅ is V, A, G, Y, R, I, W, P, C or S;
U₆ is Y, R, V, W, I, V, S, N, P, or D;
U₇ is W, S, Y, V, R, I, A, or P;
U₈ is 5, Y, K, A, P, W, R, N, or D; and
U₉ is G, S, A, P, W, R, Y, I, V, or K.

8. The polypeptide of claim 5, comprising the sequence of any one of SEQ ID NOS: 59, 61-66, 68-76, and 78-80, or a sequence that is at least 90%, at least 91%, at least 92%, at least 93%, at least 94%, at least 95%, at least 96%, at least 97%, at least 98%, or at least 99% identical to any one of SEQ ID NOS: 59, 61-66, 68-76, and 78-80.

9. A polypeptide comprising a sequence ATGVRAVPGNENSLEIEELARFAVDEHNKKENALLEFVRB₉VKAKEQZ₁Z₂Z₃Z₄Z₅Z₆Z₇Z₈Z₉TMYYLTLEAKDGGKKKLYEAKVWVKU₁U₂U₃U₄U₅U₆U₇U₈U₉NFKELQEFKB₁₀VGDA, wherein each of Z₁-Z₉, each of B₉-B₁₀, and each of U₁-U₉ are independently an amino acid (SEQ ID NO: 105), provided that at least 1, 2, 3, 4, or 5 amino acids of Z₁-Z₉ and U₁-U₉ do not correspond to the amino acid of a wild-type affimer sequence.

10. The polypeptide of any one of claim 9, wherein (i) each of U₁-U₉ is independently I, G, A, S, V, W, R, Y, D, K, P, V, or C, or wherein each of U₁-U₉ is independently Y, G, S, C, W, R, V, I, D, P, or A; and (ii) wherein each of Z₁-Z₉ is independently Y, I, S, V, D, K, W, A, R, or N.

11. The polypeptide of claim 9, wherein
U₁ is Y or W;
U₂ is G or V;
U₃ is G or I;
U₄ is S or R;
U₅ is C or G;
U₆ is W or D;
U₇ is R or S;
U₈ is G or P;
U₉ is G or A;
Z₁ is Y or R;
Z₂ is I or A;
Z₃ is S or R;
Z₄ is V or Y;
Z₅ is D or Y;
Z₆ is K or Y;
Z₇ is W or V;
Z₈ is V or N;
Z₉ is A or R;
B₉ is V or M; and
B₁₀ is P or L

12. The polypeptide of claim 9, comprising the sequence of any one of SEQ ID NOS: 82-83 and 106, or a sequence that is at least 90%, at least 91%, at least 92%, at least 93%, at least 94%, at least 95%, at least 96%, at least 97%, at least 98%, or at least 99% identical to any one of SEQ ID NOS: 82-83 and 106.

13. A polypeptide comprising a sequence ATGVRAB₁B₂B₆NENB₃LEIEELARFAVDEHNKKENALLEFVB₇VB₈KAKEQVVAB₄TMYYLTLEAKDB₁₁GKKKLYEAKVWVKU₁U₂U₃U₄U₅U₆U₇U₈U₉NFB₅ELQEFKPVGDA, wherein each of B₁-B₈ and U₁-U₉ are independently an amino acid, provided that at least 1, 2, 3, 4, or 5 amino acids of B₁-B₄ and U₁-U₉ do not correspond to the amino acid of a wild-type affimer sequence.

14. The polypeptide of claim 13, wherein:
B₁ is M or V;
B₂ is S or P;
B₃ is G or S;
B₄ is C or G;
B₅ is R or K;
B₆ is G or S;
B₇ is R or C;
B₈ is V or M;
B₁₁ is G or D;
U₁ is R, S, or V;
U₂ is I, R, P, or W;
U₃ is V or W;
U₄ is R, Y, or V;
U₅ is V or W;
U₆ is A, I, P, or S;
U₇ is V, R, K, or D;
U₈ is W, G, or P; and
U₉ is V, G, or A.

15. The polypeptide of claim 13, wherein each of U₁-U₉ is independently I, G, A, S, V, W, R, Y, D, K, P, V, or C; or wherein each of U₁-U₉ is independently R, I, V, A, W, S, P, Y, K, or G.

16. The polypeptide of claim 13, comprising the sequence of any one of SEQ ID NOS: 85-88, 60, 67, and 77, or a sequence that is at least 90%, at least 91%, at least 92%, at least 93%, at least 94%, at least 95%, at least 96%, at least 97%, at least 98%, or at least 99% identical to any one of SEQ ID NOS: 85-88, 60, 67, and 77.

17. A polypeptide comprising a sequence VDNKFNKEJ₁J₂J₃AJ₄J₅EIJ₆J₇LPNLNJ₈J₉QJ₁₀J₁₁AFIJ₁₂SJ₁₇J₁₃DDPJ₁₄J₁₅J₁₆ANLLAEAKKL NDAQAPK, wherein each of J₁-J₁₇ is independently an amino acid or absent (SEQ ID NO: 108, provided that at least 1, 2, 3, 4, or 5 amino acids of J₁-J₁₇ do not correspond to the amino acid of a wild-type affibody sequence.

18. The polypeptide of claim 17, wherein each of J₁-J₁₇ are independently Q, W, L, Y, H, A, M, R, E, K, V, S, Q, R, or P.

19. The polypeptide of claim 17, wherein:
J₁ is Q, W, Y, E, L, M, K, or S;
J₂ is W, Y, S, L, A, M, Q, V, or R;
J₃ is L, A, V, M, Y, W, or H;
J₄ is W, E, M, H, V, A, or Y;
J₅ is Y, L, M, V, S, or A;
J₆ is H, W, Y, E, L, Q, A, Y, M, R, or S;
J₇ is Y, W, L, R, S, E, or A;
J₈ is A, M, S, L, or H;
J₉ is M, K, Y, Q, W, or L;
J₁₀ is H, W, L, M, E, S, A, or absent;
J₁₁ is R, Y, V, A, L, or E;
J₁₂ is L, Y, V, W, E, M, or Q;
J₁₃ is Y, E, V, R, W, Q, or H;
J₁₄ is R or S;
J₁₅ is R or Q;
J₁₆ is H, S, P, or R; and
J₁₇ is L or P.

20. The polypeptide of claim 17, comprising the sequence of any one of SEQ ID NOS: 91-103, or a sequence that is at least 90%, at least 91%, at least 92%, at least 93%, at least 94%, at least 95%, at least 96%, at least 97%, at least 98%, or at least 99% identical to any one of SEQ ID NOS: 91-103.

21. (canceled)
